(19) United States
(12) Patent Application Publication
(10) Pub. No.: US 2023/0170096 A1
(43) Pub. Date: Jun. 1, 2023

(54) SYSTEMS, DEVICES, AND METHODS FOR MANAGING CONTACT INSTANCES OF PERSONS OF INTEREST

(71) Applicant: Vasan Abe SUN, Bangkok (TH)

(72) Inventors: Vasan Abe SUN, Bangkok (TH); Warner SUN, Bangkok (TH)

(21) Appl. No.: 17/997,986

(22) PCT Filed: May 5, 2021
(86) PCT No.: PCT/SG2021/050247

§ 371 Date: Nov. 4, 2022

(30) Foreign Application Priority Data
May 6, 2020 (SG) 10202004179V
May 6, 2020 (SG) 10202013127S

(51) Int. Cl. G16H 50/30; H04W 4/02; H04W 4/029; G06F 16/29

(52) U.S. Cl. CPC G16H 50/30; H04W 4/023; H04W 4/029; G06F 16/29

(57) ABSTRACT

Embodiments relate to systems and methods for managing contact instances. The method includes receiving a candidate contact instance. The candidate contact instance includes first and second information sets for first and second users, respectively. First information set includes a geolocation, timestamp, and first user identifier. Second information set include a geolocation, timestamp, and second user identifier. The method includes performing an exposure assessment process for the candidate contact instance, including deriving a non-contact score. Non-contact score represents a likelihood the first and second users were not physically exposed to one another during the candidate contact instance. Non-contact score is derived in one or more ways, including performing a layout analysis, map analysis, transportation analyses, and/or different levels analysis. The method includes selecting the candidate contact instance as a contact instance in which there is a risk of physical exposure when the non-contact score is below a threshold value.

### TECHNICAL FIELD

The present disclosure relates generally to managing contact instances between users, and more specifically, to methods, systems, devices, and logic for managing potential physical exposure between users from among a plurality of contact instances between users.

### BACKGROUND

Infectious and/or contagious diseases caused by viruses, bacteria, other infectious agents, contagions, radiation, etc. have become increasingly prevalent and widespread over the years. With more densely populated cities, ever-growing use of crowded public transportation, increasing popularity of shopping malls, sports stadiums/arenas and other public areas, and ease, availability, and affordability of international travel, the “spreading” of such infectious and/or contagious diseases throughout and across cities, states/provinces, countries, regions, and continents has also become increasingly difficult to control and/or stop.

### BRIEF SUMMARY

One of the main approaches to controlling the spread of infectious and/or contagious diseases, such as the COVID-19 disease, is to perform contact tracing of persons of interest. Conventional approaches to performing contact tracing involve gathering as much information as possible from an infected person (e.g., a person who has tested positive for a virus (e.g., severe acute respiratory syndrome coronavirus 2, or SARS-CoV-2), bacteria, and/or disease, and who may have been contagious or capable of spreading such viruses, bacteria, and/or diseases to others prior to being identified as being infected). Responsible authorities will then “manually” trace back to each person the infected person has been in contact with and take action (e.g., notify the person, test the person to determine whether the person has been infected, quarantine the person if the person has tested positive, and/or require the person to be in isolation for a certain period of time (e.g., stay at home, stay at a facility, etc. for 14 days)). Responsible authorities will also “manually” trace back to each place/location the infected person has visited over a certain period of time (e.g., 14 days) to identify persons who were at the place/location at the same date/time as the infected person (or within a certain period of time thereafter, such as 1-3 days) and take action (as described above). While such a manual approach is widely used globally and is presently one of the most effective ways of performing contact tracing, such an approach is not without problems. For example, such an approach relies heavily on the infected person being able to accurately, correctly, and completely recollect (or remember or recall) where he/she has visited and who he/she has interacted with (i.e., potential physical exposure) over a certain time period (e.g., 14 days). In situations where the infected person does not remember (or chooses not to remember) some people or places/locations, infectious and/or contagious diseases can quickly spread throughout and across cities, states/provinces, countries, regions, and continents.

In an exemplary embodiment, a method of managing contact tracing information is described. The method includes receiving, by a processor, a candidate contact instance, the candidate contact instance including: a first candidate first user information set for a first user device of a first user, the first candidate first user information set including a geolocation of the first user device and a timestamp representing a time when the geolocation of the first user device was recorded; and a first candidate second user information set for a second user device of a second user, the first candidate second user information set including a geolocation of the second user device and a timestamp representing a time when the geolocation of the second user device was recorded. The method includes performing, by the processor, a physical exposure assessment process for the candidate contact instance, the physical exposure assessment process including: deriving a first non-contact score for the candidate contact instance, the first non-contact score for the candidate contact instance representing a likelihood that the first and second users were not physically exposed to one another during the candidate contact instance. The deriving of the first non-contact score includes performing a layout analysis for the candidate contact instance. The layout analysis for the candidate contact instance includes: searching for a first floor layout, the first floor layout being a floor layout for a geographical area covering the geolocation in the first candidate first user information set and the geolocation in the first candidate second user information set; identifying a first location on the first floor layout representing the geolocation in the first candidate first user information set; identifying a second location on the first floor layout representing the geolocation in the first candidate second user information set; analyzing a first area on the first floor layout, the first area on the first floor layout including at least an area between the first and second locations on the first floor layout, the analyzing of the first area on the first floor layout including determining a likelihood that a first physical barrier is represented in the first area on the first floor layout; and deriving a first physical barrier likelihood, the first physical barrier likelihood being a likelihood that the first physical barrier was present between the geolocation in the first candidate first user information set and the geolocation in the first candidate second user information set during the candidate contact instance, the first physical barrier likelihood derived based on the analyzing of the first area on the first floor layout. The deriving of the first-non-contact score may also include performing a first map analysis for the candidate contact instance. The first map analysis for the candidate contact instance includes: searching for a first map, the first map being a map for a geographical area covering the geolocation in the first candidate first user information set and the geolocation in the first candidate second user information set; identifying a first location on the first map representing the geolocation in the first candidate first user information set; identifying a second location on the first map representing the geolocation in the first candidate first second information set; analyzing a first area on the first map, the first area on the first map including at least an area between the first and second locations on the first map, the analyzing of the first area on the first map including determining a likelihood that a second physical barrier is represented in the first area on the first map; and deriving a second physical barrier likelihood, the second physical barrier likelihood being a likelihood that the second physical barrier was present between the geolocation in the first candidate first user information set and the geolocation in the first candidate second user information set during the candidate contact instance, the second physical barrier likelihood derived based on the analyzing of the first area on the first map. The first non-contact score for the candidate contact instance is derived based on at least the first physical barrier likelihood and/or the second physical barrier likelihood. The physical exposure assessment process also includes selecting the candidate contact instance as a contact instance in which there is a risk of physical exposure when the first non-contact score is below a threshold non-contact score.

The method includes identifying, by the processor, two consecutive first user information sets consecutively generated by the first user device, the two consecutive first user information sets including a first consecutive first user information set and a second consecutive first user information set, the first consecutive first user information set having a first geolocation and a first timestamp, the second consecutive first user information set having a second geolocation and a second timestamp. The method includes generating, by the processor, one or more first interpolated first user information sets, each first interpolated first user information set having a first interpolated timestamp and a first interpolated geolocation, each first interpolated timestamp being a timestamp selected between the first timestamp and the second timestamp, each first interpolated geolocation being a geolocation between the first geolocation and the second geolocation that has been interpolated based on at least the first interpolated timestamp. The method includes identifying, by the processor, two consecutive second user information sets that have been consecutively generated by the second user device, the two consecutive second user information sets including a first consecutive second user information set and a second consecutive second user information set, the first consecutive second user information set having a third geolocation and a third timestamp, the second consecutive second user information set having a fourth geolocation and a fourth timestamp. The method includes generating, by the processor, one or more first interpolated second user information sets, each first interpolated second user information set having a second interpolated timestamp and a second interpolated geolocation, each second interpolated timestamp being a timestamp selected between the third timestamp and the fourth timestamp, each second interpolated geolocation being a geolocation between the third geolocation and the fourth geolocation that has been interpolated based on at least the second interpolated timestamp. The first geolocation and the first timestamp of the first consecutive first user information set is generated by the first user device. The second geolocation and the second timestamp of the second consecutive first user information set is generated by the first user device. The third geolocation and the third timestamp of the first consecutive second user information set is generated by the second user device. The fourth geolocation and the fourth timestamp of the second consecutive second user information set is generated by the second user device. The first interpolated geolocation and the first interpolated timestamp of each first interpolated first user information set is generated by the processor and not the first user device. The second interpolated geolocation and the second interpolated timestamp of each first interpolated second user information set is generated by the processor and not the second user device. One or more of the following may apply: the first candidate first user information set is one of the first interpolated first user information sets generated by the processor; and/or the first candidate second user information set is one of the first interpolated second user information sets generated by the processor.

The physical exposure assessment process includes deriving an inaccuracy score for each of the one or more candidate contact instances, including a first inaccuracy score for the first candidate contact instance. The first inaccuracy score for the first candidate contact instance represents a likelihood that: the distance between the geolocation in the first candidate first user information set and the geolocation in the first candidate first other user information set is not less than the threshold distance value; and/or the amount of time between the timestamp in the first candidate first user information set and the timestamp in the first candidate first other user information set is not less than the threshold time value. The deriving of the first non-contact score includes an assessment of the first inaccuracy score.

The method includes selecting a lowest non-contact score for a first time period from among the non-contact scores derived for the one or more candidate contact instances, the lowest non-contact score having a highest likelihood of physical exposure between the first user and the first other user during the first time period. The method includes generating an overall contact likelihood for the first time period, the overall contact likelihood being an overall likelihood that the first user and the first other user were physically exposed to one another during the first time period, the overall contact likelihood generated based on the lowest non-contact score. When the lowest non-contact score is high, the overall contact likelihood is low. When the lowest non-contact score is low, the overall contact likelihood is high. The method may also include receiving a request to perform a contact trace for the first user, the first user being identified as a particular user of interest. Responsive to a determination that the overall contact likelihood is high, the method may include generating a first notification to the first other user, the first notification being a notification indicating the first other user has a high risk of having been physically exposed to the first user; and/or generating a second notification to the first user, the second notification being a notification indicating the first user has a high risk of having been physically exposed to the first other user; and/or generating a third notification to a trusted authority, the third notification being a notification indicating the first user and the first other user have a high risk of having been physically exposed to one another, the third notification including one or more information of the candidate contact instance used to derive the lowest non-contact score.

In another exemplary embodiment, a method of managing contact tracing information is described. The method includes receiving, by a processor, a candidate contact instance, the candidate contact instance including: a first candidate first user information set for a first user device of a first user, the first candidate first user information set including a geolocation of the first user device and a timestamp representing a time when the geolocation of the first user device was recorded; and a first candidate second user information set for a second user device of a second user, the first candidate second user information set including a geolocation of the second user device and a timestamp representing a time when the geolocation of the second user device was recorded. The method includes performing, by the processor, a physical exposure assessment process for the candidate contact instance. The physical exposure assessment process includes deriving a first non-contact score for the candidate contact instance, the first non-contact score for the candidate contact instance representing a likelihood that the first and second users were not physically exposed to one another during the candidate contact instance. The deriving of the first non-contact score includes performing a first transportation-based separation analysis for the candidate contact instance, the first transportation-based separation analysis for the candidate contact instance including searching for a first map, the first map being a map for a geographical area covering the geolocations of the first candidate first user information set and the first candidate second user information set; deriving a first transport area likelihood, the first transport area likelihood being a likelihood that the geolocation in the first candidate first user information set is within a first transport area on the first map, the first transport area being an area on the first map representing an area intended as a vehicle transportation area; deriving a second transport area likelihood, the second transport area likelihood being a likelihood that the geolocation in the first candidate second user information set is within a second transport area on the first map, the second transport area being an area on the first map representing an area intended as a vehicle transportation area; and deriving a vehicular separation likelihood, the vehicular separation likelihood being a likelihood that the first user and/or the second user were in a vehicle during the candidate contact instance, the vehicular separation likelihood derived based on the first transport area likelihood and the second transport area likelihood. The deriving of the first non-contact score includes performing a second transportation-based separation analysis for the candidate contact instance, the second transportation-based separation analysis for the candidate contact instance including: obtaining one or more previous first user information sets and/or one or more subsequent first user information sets, wherein a timestamp of each of the one or more previous first user information sets are timestamps that precede the timestamp of the first candidate first user information set, wherein a timestamp of each of the one or more subsequent first user information sets are timestamps that are after the timestamp of the first candidate first user information set; obtaining one or more previous second user information sets and/or one or more subsequent second user information sets, wherein a timestamp of each of the one or more previous second user information sets are timestamps that precede the timestamp of the first candidate second user information set, wherein a timestamp of each of the one or more subsequent second user information sets are timestamps that are after the timestamp of the first candidate second user information set; deriving a travel speed of the first user device based on the first candidate first user information set, the one or more previous first user information sets, and/or the one or more subsequent first user information sets; deriving a travel speed of the second user device based on the first candidate second user information set, the one or more previous second user information sets, and/or the one or more subsequent second user information sets; and deriving a first different transport mode likelihood, the first different transport mode likelihood being a likelihood that the first user and the second user were traveling via a different transport mode during the candidate contact instance, the first different transport mode likelihood derived based on a comparison of the derived travel speed of the first user device and the derived travel speed of the second user device. The deriving of the first non-contact score includes performing a third transportation-based separation analysis for the candidate contact instance, the third transportation-based separation analysis for the candidate contact instance including: obtaining the one or more previous first user information sets and/or the one or more subsequent first user information sets; obtaining the one or more previous second user information sets and/or the one or more subsequent second user information sets; deriving a travel direction of the first user device based on the first candidate first user information set, the one or more previous first user information sets, and/or the one or more subsequent first user information sets; deriving a travel direction of the second user device based on the first candidate second user information set, the one or more previous second user information sets, and/or the one or more subsequent second user information sets; and deriving a second different transport mode likelihood, the second different transport mode likelihood being a likelihood that the first user and the second user were traveling via a different transport mode during the candidate contact instance, the second different transport mode likelihood derived based on a comparison of the derived travel direction of the first user device and the derived travel direction of the second user device. The first non-contact score for the candidate contact instance is derived based on at least the vehicular separation likelihood, the first different transport mode likelihood, and/or the second different transport mode likelihood. The physical exposure assessment process includes selecting the candidate contact instance as a contact instance in which there is a risk of physical exposure when the first non-contact score is below a threshold non-contact score.

The method includes identifying, by the processor, two consecutive first user information sets consecutively generated by the first user device, the two consecutive first user information sets including a first consecutive first user information set and a second consecutive first user information set, the first consecutive first user information set having a first geolocation and a first timestamp, the second consecutive first user information set having a second geolocation and a second timestamp. The method includes generating, by the processor, one or more first interpolated first user information sets, each first interpolated first user information set having a first interpolated timestamp and a first interpolated geolocation, each first interpolated timestamp being a timestamp selected between the first timestamp and the second timestamp, each first interpolated geolocation being a geolocation between the first geolocation and the second geolocation that has been interpolated based on at least the first interpolated timestamp. The method includes identifying, by the processor, two consecutive second user information sets that have been consecutively generated by the second user device, the two consecutive second user information sets including a first consecutive second user information set and a second consecutive second user information set, the first consecutive second user information set having a third geolocation and a third timestamp, the second consecutive second user information set having a fourth geolocation and a fourth timestamp. The method includes generating, by the processor, one or more first interpolated second user information sets, each first interpolated second user information set having a second interpolated timestamp and a second interpolated geolocation, each second interpolated timestamp being a timestamp selected between the third timestamp and the fourth timestamp, each second interpolated geolocation being a geolocation between the third geolocation and the fourth geolocation that has been interpolated based on at least the second interpolated timestamp. The first geolocation and the first timestamp of the first consecutive first user information set is generated by the first user device. The second geolocation and the second timestamp of the second consecutive first user information set is generated by the first user device. The third geolocation and the third timestamp of the first consecutive second user information set is generated by the second user device. The fourth geolocation and the fourth timestamp of the second consecutive second user information set is generated by the second user device. The first interpolated geolocation and the first interpolated timestamp of each first interpolated first user information set is generated by the processor and not the first user device. The second interpolated geolocation and the second interpolated timestamp of each first interpolated second user information set is generated by the processor and not the second user device. One or more of the following may apply: the first candidate first user information set is one of the first interpolated first user information sets generated by the processor; and/or the first candidate second user information set is one of the first interpolated second user information sets generated by the processor.

In another exemplary embodiment, a method of managing contact tracing information is described. The method includes receiving, by a processor, a candidate contact instance, the candidate contact instance including: a first candidate first user information set for the first user, the first candidate first user information set including a geolocation and corresponding timestamp for the first user; and a first candidate second user information set for the second user, the first candidate second user information set including a geolocation and corresponding timestamp for the second user. The method includes performing, by the processor, a physical exposure assessment process for the candidate contact instance. The physical exposure assessment process includes deriving a first non-contact score for the candidate contact instance, the first non-contact score for the candidate contact instance representing a likelihood that the second user was not physically exposed to the first user during the candidate contact instance. The first non-contact score derived is based on: performing an analysis to determine whether the geolocation of the first candidate first user information set and the geolocation of the first candidate second user information set are in a multi-level building; and responsive to a determination that the geolocation of the first candidate first user information set and the geolocation of the first candidate second user information set are in a multi-level building: searching, in a floor layout database, for one or more floor layouts based on the geolocation of the first candidate first user information set and the geolocation of the first candidate second user information set; for each of the one or more floor layouts: identifying a first location on the floor layout representing the geolocation in the first candidate first user information set; identifying a second location on the floor layout representing the geolocation in the first candidate second user information set; identifying one or more other first locations on the floor layout based on one or more other first user information sets, the one or more other first locations including: one or more previous first locations on the floor layout, the one or more previous first locations representing one or more locations of the first user prior to the first location; and/or one or more subsequent first locations on the floor layout, the one or more subsequent first locations representing one or more locations of the first user subsequent to the first location; and identifying one or more other second locations on the floor layout based on one or more other second user information sets, the one or more other second locations including: one or more previous second locations on the floor layout, the one or more previous second locations representing one or more locations of the second user prior to the second location; and/or one or more subsequent second locations on the floor layout, the one or more subsequent second locations representing one or more locations of the second user subsequent to the second location; selecting, based on the identifying of the first location and the one or more other first locations, one or more first select floor layouts, each first select floor layout being a floor layout in which there is no physical barrier found, by the processor, in a travel path of the first user, the travel path of the first user determined based on the first location and the one or more other first locations; selecting, based on the identifying of the second location and the one or more other second locations, one or more second select floor layouts, each second select floor layout being a floor layout in which there is no physical barrier found, by the processor, in a travel path of the second user, the travel path of the second user determined based on the second location and the one or more other second locations; comparing the one or more first select floor layouts with the one or more second select floor layouts; and deriving a different level likelihood, the different level likelihood being a likelihood that the first user and the second user were on different levels of the multi-level building during the candidate contact instance, the different level likelihood derived based on the comparing of the one or more first select floor layouts with the one or more second select floor layouts. The first non-contact score for the candidate contact instance is determined based on the different level likelihood. The physical exposure assessment process includes identifying the second user as having a risk of being physically exposed to the first user when the first non-contact score is below a threshold non-contact score.

The method includes identifying, by the processor, two consecutive first user information sets consecutively generated by the first user device, the two consecutive first user information sets including a first consecutive first user information set and a second consecutive first user information set, the first consecutive first user information set having a first geolocation and a first timestamp, the second consecutive first user information set having a second geolocation and a second timestamp. The method includes generating, by the processor, one or more first interpolated first user information sets, each first interpolated first user information set having a first interpolated timestamp and a first interpolated geolocation, each first interpolated timestamp being a timestamp selected between the first timestamp and the second timestamp, each first interpolated geolocation being a geolocation between the first geolocation and the second geolocation that has been interpolated based on at least the first interpolated timestamp. The method includes identifying, by the processor, two consecutive second user information sets that have been consecutively generated by the second user device, the two consecutive second user information sets including a first consecutive second user information set and a second consecutive second user information set, the first consecutive second user information set having a third geolocation and a third timestamp, the second consecutive second user information set having a fourth geolocation and a fourth timestamp. The method includes generating, by the processor, one or more first interpolated second user information sets, each first interpolated second user information set having a second interpolated timestamp and a second interpolated geolocation, each second interpolated timestamp being a timestamp selected between the third timestamp and the fourth timestamp, each second interpolated geolocation being a geolocation between the third geolocation and the fourth geolocation that has been interpolated based on at least the second interpolated timestamp. The first geolocation and the first timestamp of the first consecutive first user information set is generated by the first user device. The second geolocation and the second timestamp of the second consecutive first user information set is generated by the first user device. The third geolocation and the third timestamp of the first consecutive second user information set is generated by the second user device. The fourth geolocation and the fourth timestamp of the second consecutive second user information set is generated by the second user device. The first interpolated geolocation and the first interpolated timestamp of each first interpolated first user information set is generated by the processor and not the first user device. The second interpolated geolocation and the second interpolated timestamp of each first interpolated second user information set is generated by the processor and not the second user device. One or more of the following may apply: the first candidate first user information set is one of the first interpolated first user information sets generated by the processor; and/or the first candidate second user information set is one of the first interpolated second user information sets generated by the processor.

In another exemplary embodiment, a method of managing contact tracing information is described. The method includes receiving, by a processor from a first mobile device of a first user, a plurality of first user information sets, each of the first user information sets including a geolocation of the first mobile device and a corresponding timestamp for the geolocation of the first mobile device. The method includes receiving, by the processor from a second mobile device of a second user, a plurality of second user information sets, each second user information set including a geolocation of the second mobile device and a corresponding timestamp for the geolocation of the second mobile device. The method includes performing, by the processor, a candidate contact process. The candidate contact process includes selecting a first candidate first user information set and a first candidate second user information set from among the plurality of first user information sets and the plurality of second user information sets, respectively, based on at least the following conditions: a distance between a geolocation in the first candidate first user information set and a geolocation in the first candidate second user information set is less than a threshold distance value; and an amount of time between a timestamp in the first candidate first user information set and a timestamp in the first candidate second user information set is less than a threshold time value. The candidate contact process includes generating a first candidate contact instance, the first candidate contact instance including the first candidate first user information set and the first candidate second user information set. The method includes performing, by the processor, a physical exposure assessment process for the first candidate contact instance, the physical exposure assessment process including: deriving a first non-contact score for the first candidate contact instance, the first non-contact score for the first candidate contact instance representing a likelihood that the second user was not physically exposed to the first user during the first candidate contact instance, the first non-contact score derived based on an assessment of whether or not a physical barrier was present between the geolocation in the first candidate first user information set and the geolocation in the first candidate second user information set during the first candidate contact instance; and selecting the first candidate contact instance as a contact instance in which there is a risk of physical exposure when the first non-contact score is below a threshold non-contact score.

In another exemplary embodiment, a method of managing contact tracing information is described. The method includes receiving, by a processor from a first mobile device of a first user, a plurality of first user information sets, each of the first user information sets including a geolocation of the first mobile device and a corresponding timestamp for the geolocation of the first mobile device. The method includes identifying, by the processor, two consecutive first user information sets from among the plurality of first user information sets, the two consecutive first user information sets consecutively generated by the first user device, the two consecutive first user information sets including a first consecutive first user information set and a second consecutive first user information set, the first consecutive first user information set having a first geolocation and a first timestamp, the second consecutive first user information set having a second geolocation and a second timestamp. The method includes generating, by the processor, a first interpolated first user information set, the first interpolated first user information set having an interpolated timestamp and an interpolated geolocation, the interpolated timestamp being a timestamp between the first timestamp and the second timestamp, the interpolated geolocation being a geolocation selected based on at least the first interpolated timestamp. The method includes receiving, by the processor from a second mobile device of a second user, a plurality of second user information sets, each of the second user information sets including a geolocation of the second mobile device and a corresponding timestamp for the geolocation of the second mobile device. The method includes performing, by the processor, a candidate contact process, the candidate contact process including: selecting the first interpolated first user information set and a first candidate second user information set from among the plurality of second user information sets when the following conditions are met: a distance between the interpolated geolocation in the first interpolated first user information set and a geolocation in the first candidate second user information set is less than a threshold distance value; and an amount of time between the interpolated timestamp in the first interpolated first user information set and a timestamp in the first candidate second user information set is less than a threshold time value; and generating a first candidate contact instance, the first candidate contact instance including the first interpolated first user information set and the first candidate second user information set.

In another exemplary embodiment, a method of managing contact tracing information is described. The method includes receiving, by a processor, a candidate contact instance, the candidate contact instance including: a first candidate first user information set for a first user device of a first user, the first candidate first user information set including a geolocation, a corresponding timestamp, and an anonymized first user identifier for the first user; and a first candidate second user information set for a second user device of a second user, the first candidate second user information set including a geolocation, a corresponding timestamp, and an anonymized second user identifier for the second user. The method includes performing, by the processor, a physical exposure assessment process for the candidate contact instance, the physical exposure assessment process including: deriving a first non-contact score for the candidate contact instance, the first non-contact score for the candidate contact instance representing a likelihood that the second user was not physically exposed to the first user during the candidate contact instance. The first non-contact score is derived based at least a layout analysis for the candidate contact instance. The layout analysis for the candidate contact instance includes: searching for a first floor layout, the searching based on the geolocations of the first candidate first user information set and the first candidate second user information set; locating, on the first floor layout, the geolocations of the first candidate first user information set and the first candidate second user information set; and deriving, based on the locating of the geolocations of the first candidate first user information set and the first candidate second user information set on the first floor layout, a first likelihood, wherein the first likelihood is a likelihood of a presence of a physical barrier between the geolocations of the first candidate first user information set and the first candidate second user information set. The first non-contact score for the candidate contact instance is determined based on at least the first likelihood. The physical exposure assessment process includes selecting the candidate contact instance as a contact instance in which there is a risk of physical exposure when the first non-contact score is below a threshold non-contact score.

### BRIEF DESCRIPTION OF THE FIGURES

For a more complete understanding of the present disclosure, example embodiments, and their advantages, reference is now made to the following description taken in conjunction with the accompanying figures, in which like reference numbers indicate like features, and:

FIG. 1A is an illustration of an example embodiment of a system for managing contact tracing information;

FIG. 1B is an illustration of an example embodiment of an information set for a user;

FIG. 1C is an illustration of an example embodiment of an information set;

FIG. 1D is an illustration of an example embodiment of a candidate contact instance;

FIG. 2 is an illustration of an example embodiment of a processor;

FIG. 3 is an illustration of an example embodiment of a threshold processor;

FIG. 4 is an illustration of an example embodiment of a candidate contact processor;

FIG. 5 is an illustration of an example embodiment of a physical exposure assessment processor;

FIG. 6 is an illustration of an example embodiment of a non-contact processor;

FIG. 7A is an illustration of an example embodiment of performing a layout analysis based on user information sets and a floor layout;

FIG. 7B is an illustration of an example embodiment of performing a map analysis or first transportation-based separation analysis based on user information sets and a map;

FIG. 7C is an illustration of an example embodiment of performing a second transportation-based separation analysis based on user information sets, previous user information sets, and/or subsequent user information sets;

FIG. 7D is an illustration of an example embodiment of performing a third transportation-based separation analysis based on user information sets, previous user information sets, and/or subsequent user information sets;

FIG. 7E and FIG. 7F illustrate an example embodiment of performing a different level analysis based on user information sets, previous user information sets, and/or subsequent user information sets;

FIG. 7G is an illustration of an example embodiment of generating an interpolated geolocation; and

FIG. 7H is an illustration of an example embodiment of generating candidate contact instances with an interpolated geolocation.

Although similar reference numbers may be used to refer to similar elements in the figures for convenience, it can be appreciated that each of the various example embodiments may be considered to be distinct variations.

Example embodiments will now be described with reference to the accompanying figures, which form a part of the present disclosure and which illustrate example embodiments which may be practiced. As used in the present disclosure and the appended claims, the terms “embodiment,” “example embodiment,” “exemplary embodiment,” and “present embodiment” do not necessarily refer to a single embodiment, although they may, and various example embodiments may be readily combined and/or interchanged without departing from the scope or spirit of example embodiments. Furthermore, the terminology as used in the present disclosure and the appended claims is for the purpose of describing example embodiments only and is not intended to be limitations. In this respect, as used in the present disclosure and the appended claims, the term “in” may include “in” and “on,” and the terms “a,” “an,” and “the” may include singular and plural references. Furthermore, as used in the present disclosure and the appended claims, the term “by” may also mean “from,” depending on the context. Furthermore, as used in the present disclosure and the appended claims, the term “if” may also mean “when” or “upon,” depending on the context. Furthermore, as used in the present disclosure and appended claims, the words “and/or” may refer to and encompass any or all possible combinations of one or more of the associated listed items.

### DETAILED DESCRIPTION

Presently, “manual” contact tracing efforts appear to be the most effective ways of controlling the spread of infectious and/or contagious diseases (e.g., the COVID-19 disease caused by the severe acute respiratory syndrome coronavirus 2, or SARS-CoV-2). Such conventional approaches to contact tracing involve having the infected person or person of interest (e.g., a person who has tested positive for a virus, bacteria, and/or disease, and who may have been contagious or capable of spreading such viruses, bacteria, and/or diseases to others prior to being identified as being infected; also referred to as particular user) recollect as much information as possible about who the infected person has been in contact with and where the infected person has visited over a certain period of time (e.g., last 14 days). Responsible authorities will then “manually” trace back to each person the infected person has been in contact with and take one or more actions (e.g., contact the person, test the person to determine whether the person has been infected, quarantine the person if the person has tested positive (and perform the same “manual” contact tracing on this newly identified infected person), and/or require the person to be in isolation for a period of time if the person has tested negative). Responsible authorities will also “manually” trace back to each place/location the infected person has visited over a period of time (e.g., 14 days) to identify persons who were or may have been at the place/location at the same date/time (based on recollections from the infected person) as the infected person (or within a period of time thereafter, such as 1-3 days) and take action (as described above). While such a manual approach is widely used globally and is presently one of the most effective ways of performing contact tracing, such an approach is not without problems. For example, such an approach relies heavily on the infected person being able to accurately and correctly recollect (or remember or recall) where he/she has visited, who he/she has interacted with (i.e., potential physical exposure), and date/time of each visit and interaction over a certain period of time (e.g., last 14 days). In situations where the infected person does not remember, inaccurately remembers (e.g., wrong date/time, etc.), and/or incorrectly remembers (e.g., wrong person(s), wrong place(s)/location(s), etc.) even a single person, place/location, and/or date/time, infectious and/or contagious diseases can quickly and devastatingly spread throughout and across cities, states/provinces, countries, regions, and continents.

Present example embodiments relate generally to and/or include systems, subsystems, processors, devices, logic, methods, and processes for addressing conventional problems, including those described above and in the present disclosure, and more specifically, example embodiments relate to systems, subsystems, processors, devices, logic, methods, and processes for managing contact instances between users and/or contact tracing information, including, but not limited to, processing of geolocation and date/time stamps of users, processing of user information sets, processing of location information sets, processing of candidate contact instances for or between users, processing of false positive likelihoods for candidate contact instances, processing of physical exposure likelihoods for candidate contact instances, and processing of notifications, warnings, and/or alerts to users and/or trusted authorities.

As described in the present disclosure, an example embodiment of candidate contact instances may be generated in a plurality of ways. For example, a first user device for a first user (having Bluetooth enabled, or the like) may perform a “handshake” (e.g., exchange unique identifiers and/or other information) with a second user device of a second user (also having Bluetooth enabled, or the like) when the user devices are within a certain distance from each other (i.e., within range for Bluetooth communications, or the like). In performing such a handshake, the first device may be configurable or configured to store information pertaining to the second user (i.e., second device), and the second device may be configurable or configured to store information pertaining to the first user (i.e., first device). Such handshake may also include having each device store a timestamp. In an example embodiment, the first device may be configurable or configured to store a current geolocation (geolocation of itself; e.g., via GPS and/or other geolocation information from cellular network, etc.), and the second device may also be configurable or configured to store a current geolocation (geolocation of itself; e.g., via GPS and/or other geolocation information from cellular network, etc.). The first device may also be configurable or configured to store a current geolocation of the second device as well (if provided by the second device; otherwise, the geolocation of itself can be used as an approximate geolocation of the second device; alternatively or in addition, the first device may approximate a geolocation and/or distance between the first device and the second device based on the Bluetooth signal strength, etc.), and the second device may also be configurable or configured to store a current geolocation of the first device as well. With such stored information, the first device then generates a candidate contact instance, which includes a first user information set (including an identity of the first user, a geolocation of the first user, and a timestamp for the geolocation of the first user) and a second user information set (including an identity of the second user, a geolocation of the second user, and a timestamp for the geolocation of the second user).

Alternatively or in addition, candidate contact instances may be generated without the need for a “handshake”. As described in the present disclosure, user information sets (which include an identity of the user, a geolocation of the user, and a timestamp for the geolocation of the user) may be generated for each user (i.e., by each user device) without the need to wait for or confirm a “handshake” or positive indication that the user devices are within a certain distance or proximity of one another. In this regard, example embodiments may enable user devices to generate their own user information sets based on their own geolocations (e.g., via GPS information received by the user device) on a continuous basis, periodic basis (e.g., every 1 second, every 2 seconds, every 3 seconds, etc.), and/or occurrence of an event (e.g., a movement by the user, as identified by via GPS, an accelerometer in the user device, wake of the user device, etc.). In this regard, user information sets can be generated independently by each user device. User information sets generated by each user device are then provided to one or more processors (e.g., processor 200 described in the present disclosure) for further processing, which includes performing an analysis of received user information sets from a plurality of users (i.e., from user devices) and generating candidate contact instances based on such analysis (i.e., without the need for a “handshake”).

It is recognized in the present disclosure, however, that comparing and matching of user geolocations (from received user information sets) to form candidate contact instances may result in “false positives” (e.g., situations where two users are identified to be within a close proximity of each other (i.e., a distance between the geolocations of the user devices in the user information sets is within a certain threshold) but such users are in fact not physically exposed to one another (or not full physical exposure to one another; or reduced physical exposure to one another; etc.)). Examples of false positives include, but are not limited to, users being separated by a wall; users being separated by a window; users being separated by a vehicle door (e.g., first user is in a car and second user is on a sidewalk; first user is in a car, second user is in different car, and both cars are stuck in traffic; etc.); users being separated by another user; users not separated by anything but the first user is traveling much faster than the second user (e.g., first user is riding a bike and the second user is walking on a sidewalk; first user is jogging and the second user is sitting on a park bench; etc.; such situation results in a very short period of time of physical exposure, which is recognized in the present disclosure to have a lower likelihood of physical exposure); users not separated by anything but the first user is traveling in a different direction than the second user (which is recognized in the present disclosure to have a lower likelihood of physical exposure); users separated by a floor (e.g. first user is on level/floor 1F and second user is on level/floor 2F), etc. As described in the present disclosure, an example embodiment of the processor (e.g., processor 200) is configurable or configured to, among other things, generate candidate contact instances and derive possible risks/likelihoods of physical exposure between users for one or more such situations.

It is to be understood that, while example embodiments are mostly described in the present disclosure as pertaining to contact tracing (which includes, among other things, generating information sets, candidate contact instances, inaccuracy scores, non-contact scores, and risks/likelihoods of physical exposure) for infectious and/or contagious diseases, the principles described in the present disclosure may also be applied outside of the context of contact tracing for infectious and/or contagious diseases, such as in performing tracing of persons exposed to radiation, tracing of persons who may have witnessed or been a party or victim to an event (e.g., a crime against a particular person or at a particular location, a vehicle hit-and-run on a particular person or at a particular location, a lost child, vandalism, robbery, etc.), etc., without departing from the teachings of the present disclosure.

It is also to be understood in the present disclosure that one or more elements and/or aspects of example embodiments may include and/or implement, in part or in whole, solely and/or in cooperation with other elements, using, for example, networking technologies, cloud computing, distributed ledger technology (DLT) (e.g., blockchain), artificial intelligence (AI), machine learning, deep learning, etc. For example, in one or more of the processing described in the present disclosure including, but not limited to, the layout analysis, the map analysis, the first transportation-based analysis, the second transportation-based analysis, the third transportation-based analysis, and the different levels analysis, the processor 200 (as described in the present disclosure) may include and/or cooperate with one or more forms of artificial intelligence (AI) (e.g., machine learning or deep learning via convolution neural networks for image recognition, recurrent neural networks such as long short-term memory networks (LSTM) for speech recognition, etc.) to analyze shapes, figures, images, characters, video, and/or audio in floor layouts, maps, photos, videos, etc. (each as applicable). As a more specific example, the processor 200 may include and/or cooperate with one or more convolution neural networks, or the like, that are trained (via floor layouts and/or maps) and applied to identify physical barriers (“physical barriers” are described in the present disclosure, which may include walls, partitions, windows, vehicle doors, etc.), or the like, from lines, shapes, symbols, markings, figures, characters, or the like, found in floor layouts and/or maps. The processor 200 and/or one or more forms of artificial intelligence (AI) may then derive likelihoods that such lines, shapes, symbols, markings, figures, and/or characters represent and/or refer to physical barriers, or the like. As another example, the processor 200 may include and/or cooperate with one or more convolution neural networks, or the like, that are trained (via floor layouts and/or maps) and applied to identify regions intended for transportation (e.g., roads, highways, railroad tracks, subway tracks, canals/rivers, etc.), or the like, from lines, shapes, symbols, markings, figures, characters, or the like, found in maps and/or floor layouts.

As used in the present disclosure, the terms “contact”, “contact instance”, “first contact instance”, “second contact instance”, “candidate contact instance”, “candidate first user contact instance”, “candidate second user contact instance”, “first candidate first user contact instance”, “first candidate second user contact instance”, “second candidate first user contact instance”, “second candidate second user contact instance”, “selected contact instance”, or the like, refer to an instance (and/or occurrence, situation, occasion, interaction, event, gathering, meeting, pass by, etc.) in which there is a likelihood (and/or risk, potential, possibility, probability, chance, etc.) that two or more users (and/or two or more user devices, a first user and device of a second user, etc.) were in contact with and/or within a certain distance of each other at a particular time (and which may include within a particular duration of time), as described in the present disclosure. Furthermore, as used in the present disclosure, the terms “geolocation”, “geolocation information”, “location”, “location information”, “GPS location”, or the like, when used in reference to a user and/or a device of a user, refers to location-related information for the user and/or user device, and may include, but is not limited to, information that may be used to identify a location of the user and/or user device; GPS information and/or coordinates; other forms or types of coordinates; a position or location on a map; a position or location on a floor plan; a distance and/or direction (e.g., a vector) from and/or relative to a known and/or reference GPS location and/or set of coordinates; a distance and/or direction from a known and/or reference landmark; a triangulated location (e.g., as triangulated by 2 or more signal sources, such as WiFi devices, cellular towers, user mobile devices, and/or any other signal sources); a location approximated by a signal (e.g., Bluetooth signal, NFC signal, WiFi signal, cellular signal, etc.); interpolated location and/or geolocation (e.g., based on two other geolocations); etc.

Example embodiments will now be described below with reference to the accompanying figures, which form a part of the present disclosure.

Example Embodiments of a System for Managing Contact Instances and/or Contact Tracing Information (e.g., System 100).

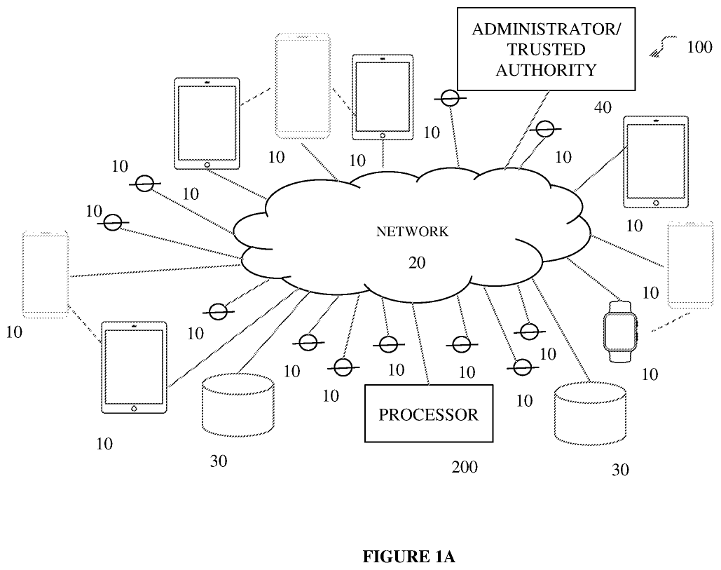

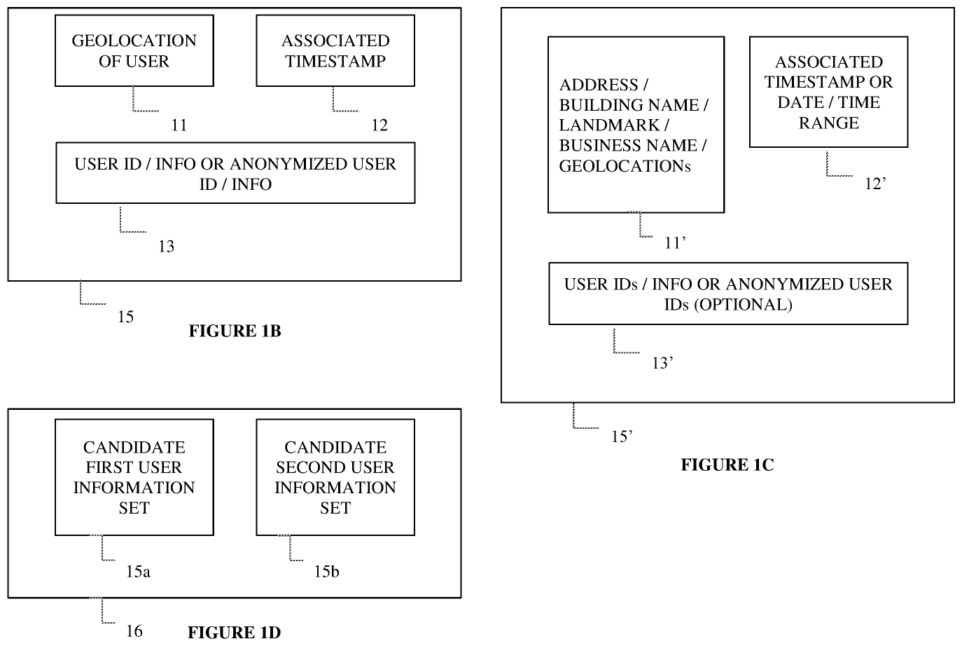

FIG. 1 illustrates an example embodiment of a system (e.g., system 100) for managing contact instances, contact tracing information, or the like. The system 100 includes one or more elements. For example, the system 100 includes one or more processors (e.g., processor 200). The system 100 may also include and/or communicate with one or more networks, communication channels, or the like (e.g., communication channels 20), which are used to enable communication between elements of the system 100. The system 100 may also include and/or communicate with one or more databases, distributed ledgers, or the like (e.g., database 30) to store, search, and/or retrieve information.

The system 100 is configurable or configured to communicate with one or more users 10, user devices 10, and/or other devices 10. As used in the present disclosure, references to a user 10 may also include a device of the user 10 (or user device 10), and vice versa. User devices 10 may include, but are not limited to, mobile phones, tablets, wearable devices (e.g., smart watches, smart glasses, smart earphones and headphones, smart speakers, etc.), etc. User devices 10 are configurable or configured to generate user information sets (e.g., see user information set 15 in FIG. 1B; as described in the present disclosure), and in some embodiments, generate candidate contact instances (e.g., see candidate contact instance 16 in FIG. 1D; as described in the present disclosure). Other devices 10 are configurable or configured to generate particular location information sets (e.g., see particular location information set 15′ in FIG. 1C; as described in the present disclosure). Examples of other devices 10 include, but are not limited to, devices fixable or installable at particular locations 11′ (e.g., a contaminated location or site, etc.) such as WiFi devices, Bluetooth devices, IoT devices, and/or any device capable of sending and/or receiving signals and/or data via the network 20. As used in the present disclosure, a user 10 who has been confirmed (e.g., by a medical practitioner, medical facility, governmental entity, etc.) as having a contagious and/or infectious disease will be referred to as “particular user” 10, “person of interest” 10, or the like. Furthermore, a user 10 who may appear to be a particular user 10 (e.g., shows certain symptoms, etc.) and/or has been physically exposed to a particular user 10 (and/or has not yet been identified or confirmed as being a particular user 10) may be referred to herein as a “potential particular user” 10, or the like.

The system 100 is also configurable or configured to communicate with one or more administrators 40 and/or trusted authorities 40 (e.g., a government entity such as a Ministry of Health, or the like). For example, communications from an administrator/trusted authority 40 to the processor 200 may include providing the processor 200 with identification 13, identifier 13, unique identifier 13, etc. of one or more particular users 10 (e.g., a person identified by medical practitioner, medical facility, Ministry of Health, or the like, after testing positive for an infectious or contagious disease; as described in the present disclosure); instructing the processor 200 to generate, based on such information 13, candidate contact instances 16; instructing the processor 200 to identify one or more other users (e.g., second users 10) who may have a risk/likelihood of physical exposure to the particular user 10 based on one or more user information sets 15 for the particular user 10, one or more user information sets 15 for the other user(s) (e.g., second users 10), one or more candidate contact instances 16, and one or more analyses described in the present disclosure (e.g., layout analysis, map analysis, first transportation-based analysis, second transportation-based analysis, third transportation-based analysis, different levels analysis, etc.); sending notifications, alerts, and/or additional information pertaining to physical exposures (physically exposed to particular users 10 and/or particular locations 11′) to administrators 40, trusted authorities 40, public bulletins or sites, particular users 10, users (e.g., second user 10) who may have been physically exposed to particular users 10 and/or particular locations 11′, and/or devices fixed or installed at particular locations 11′; identifying particular locations 11′; etc. Although the figures may illustrate the system 100 as having one processor 200, it is to be understood that the system 100 may include more than one processor 200 without departing from the teachings of the present disclosure. Each processor 200 is configurable or configured to connect to, communicate with, and/or receive information from one or more users 10, one or more user devices 10, one or more communication channels 20, one or more databases 30, and/or one or more other processors 200.

As used in the present disclosure, when applicable, a reference to a system (e.g., system 100) or processor (e.g., processor 200) may also refer to, apply to, and/or include one or more computing devices, processors, servers, systems, cloud-based computing, or the like, and/or functionality of one or more processors, computing devices, servers, systems, cloud-based computing, or the like. The system 100 and/or processor 200 (and/or its elements, as described in the present disclosure) may be any processor, server, system, device, computing device, controller, microprocessor, microcontroller, microchip, semiconductor device, or the like, configurable or configured to perform, among other things, a processing and/or managing of information, searching for information, identifying of information, data communications, and/or any one or more other actions described above and in the present disclosure. Alternatively or in addition, the system 100 and/or processor 200 (and/or its elements, as described in the present disclosure) may include and/or be a part of a virtual machine, processor, computer, node, instance, host, or machine, including those in a networked computing environment. As used in the present disclosure, a communication channel 20, network 20, cloud 20, or the like, may be or include a collection of devices and/or virtual machines connected by communication channels that facilitate communications between devices and allow for devices to share resources. Such resources may encompass any types of resources for running instances including hardware (such as servers, clients, mainframe computers, networks, network storage, data sources, memory, central processing unit time, scientific instruments, and other computing devices), as well as software, software licenses, available network services, and other non-hardware resources, or a combination thereof. A communication channel 20, network 20, cloud 20, or the like, may include, but is not limited to, the internet, intranets, WiFi systems, GPS systems, location systems, location-based service systems, computing grid systems, peer to peer systems, mesh-type systems, distributed computing environments, cloud computing environment, telephony systems, voice over IP (VoIP) systems, etc. Such communication channels 20, networks 20, clouds 20, or the like, may include hardware and software infrastructures configured to form a virtual organization comprised of multiple resources which may be in geographically disperse locations. Communication channel 20, network 20, cloud 20, or the like, may also refer to a communication medium between processes on the same device. Also as referred to herein, a network element, node, or server may be a device deployed to execute a program operating as a socket listener and may include software instances.

These and other elements of the system 100 will now be described with reference to the accompanying figures.

The Processor (e.g., Processor 200).

As illustrated in FIG. 1, the system 100 includes one or more processors (e.g., processor 200). The processor 200 is configurable or configured to manage and/or process, among other things, contact instance information, contact tracing information, or the like. For example, the processor 200 is configurable or configured to determine risks and/or likelihoods of physical exposure between users 10 (and/or between a user 10 and a particular location 11′) based on, among other things, geolocation information, temporal information, potential or candidate contact instances, confirmed or selected contact instances, and/or other information communicated by and/or for one or more elements of the system 100.

As described in the present disclosure, an example embodiment of the processor 200 is configurable or configured to receive at least geolocation and temporal information sets 15, pairs 15, packages 15, bundles 15, groups 15, payloads 15, or the like (e.g., each referred to herein as an “information set” 15, or the like) from a plurality of users 10, user devices 10, and/or other devices 10. Each user device 10 may be configurable or configured to generate such information sets 15 (e.g., continuously, periodically (e.g., every 2 seconds), intermittently, upon the occurrence of an event, etc.) and transmit such information sets 15 to the processor 200 (e.g., continuously, periodically (e.g., every 2 seconds), intermittently, upon the occurrence of an event, etc.). As illustrated in FIG. 1B, each information set 15 received from each user device 10 includes at least a geolocation 11 of the user 10 (and/or geolocation 11 of the user device 10) and a corresponding date and/or time stamp 12 (referred to herein as a “time stamp” 12, “timestamp” 12, or the like) for the geolocation of the user 10 and/or user device 10. Each information set 15 also includes identification 13, identity 13, and/or unique identifier 13 (referred to herein as a “unique identifier” 13, or the like) of the user 10 and/or user device 10. Such unique identifier 13 of the user 10 and/or user device 10 may include, but is not limited to, one or more of the following anonymized and/or non-anonymized information of and/or for the user 10 and/or user device 10: legal name 13, user ID 13, user login name 13, government-issued identification number 13 of the user 10 (e.g., national identification number 13, citizenship number 13, driver license number 13, passport number 13, etc.), mobile phone number 13 for the user device 10, email address 13 of the user 10, user personal information 13, user address 13, user employment information 13, user family information 13, user marital status 13, unique series of alphanumeric characters 13 assigned to the user 10, a combination of one or more of the aforementioned information, a hash of one or more of the aforementioned information (e.g., via SHA-256, SH-3, or the like), etc.

As illustrated in FIG. 1C, information sets 15′ may also be generated for particular locations 11′ (e.g., address, building name, landmark, business name, particular airplane, particular train or train car, particular bus, boat, taxi, or other vehicles, set of geolocations, etc.), and not just users 10. For example, in situations where a particular location 11′ is identified as having been contaminated by viruses, bacteria, other infectious agents or contagions, radiation, etc. and/or visited by one or more particular users 10, example embodiments of the processor 200 are configurable or configured to receive and/or generate one or more information set 15′ for such particular location 11′ (also referred to herein as a “particular location information set” 15′) and perform contact tracing of users 10 who have visited the particular location 11′ during a particular date/time range 12′ (e.g., within 14 days before and/or after the particular location 11′ is known to be contaminated) (referred to herein as “location-based contact tracing”). It is to be understood in the present disclosure that particular location information sets 15′ may also be generated for any location. Referring to FIG. 1C, the particular location information set 15′ for the particular location 11′ may include location-related information including, but not limited to, an address 11′, a building name 11′, a landmark name 11′, a business name 11′, a particular room 11′ or floor/level 11′, a section 11′ of a building or property/compound, a set of geolocation(s) 11′, etc., which may be used in example embodiments to perform contact tracing for any users 10 who have visited the particular location 11′ within a particular date/time range. The particular location information set 15′ for the particular location 11′ may also include temporal information, including an associated timestamp 12′ (e.g., starting date) and/or date/time range 12′ (e.g., range of dates/times starting on a date/time when the one or more particular users 10 visited the particular location 11′ until a date/time when the particular location 11′ was closed, cleaned, disinfected, etc. (or no end date/time if still ongoing)). The particular location information set 15′ for the particular location 11′ may also include unique identifier(s) 13′ (anonymized and/or non-anonymized) of one or more of the particular users 10 who have visited the particular location 11′ during a relevant date/time range or specific dates/times. It is recognized in the present disclosure that such unique identifier information 13 for users 10 may be further used by example embodiments of the processor 200 to perform contact tracing based on geolocation and temporal information of such users 10. For example, based on the particular location information set 15′, the processor 200 is able to compare the particular location information 11′ and temporal information 12′ of the particular location information set 15′ with geolocation 11 and temporal information 12 of user information sets 15 of a plurality of users 10 to identify those users 10 who may have a risk of being physically exposed to a virus, bacteria, infectious agent, contagion, radiation, etc. (or potential particular users 10). As a second step, each such particular location information set 15′ may be amended or supplemented to include a unique identifier 13 of such potential particular users 10. In addition to or alternatively, the processor 200 may perform contact tracing using the geolocation 11 and temporal information 12 of such potential particular users 10 to identify other potential particular users 10 who may have a risk of being physically exposed to the potential particular users 10 (e.g., after the potential particular users 10 visited the particular location 11′). And if such other potential particular users 10 are identified by the processor 200 as having a risk of being physically exposed to the potential particular users 10, the processor 200 may perform contact tracing using the geolocation and temporal information 121 of such other potential particular users 10 to identify other further potential particular users 10 who may have a risk of being physically exposed to such other potential particular users 10. And so on. Furthermore, for each potential particular users 10 (including the potential particular users 10, the other potential particular users 10, the other further potential particular users 10, and so on), the processor 200 may perform location-based contact tracing to identify particular locations (e.g., address, building name, landmark, business name, set of geolocations, etc.) where such potential particular users 10 have visited (e.g., based on the geolocation and temporal information of such users 10) and perform contact tracing based on such identified particular locations and the date/time of visit of the potential particular users 10. And so on. In an example embodiment, the particular location information sets 15′ may be generated by the processor 200, an administrator 40, and/or a trusted authority 40. In some example embodiments, the particular location information sets 15′ may also be generated by users 10 and submitted to the processor 200, administrator 40, and/or trusted authority 40 for checking, verification, confirmation, and/or approval.

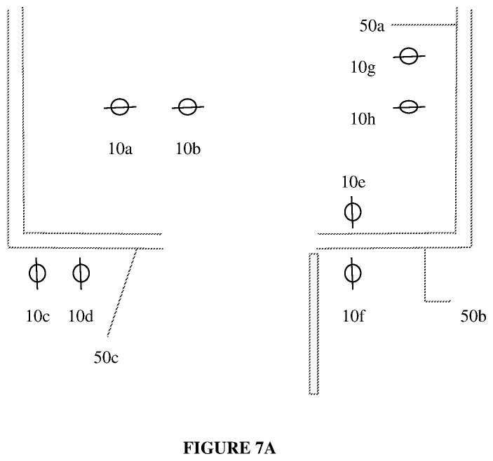

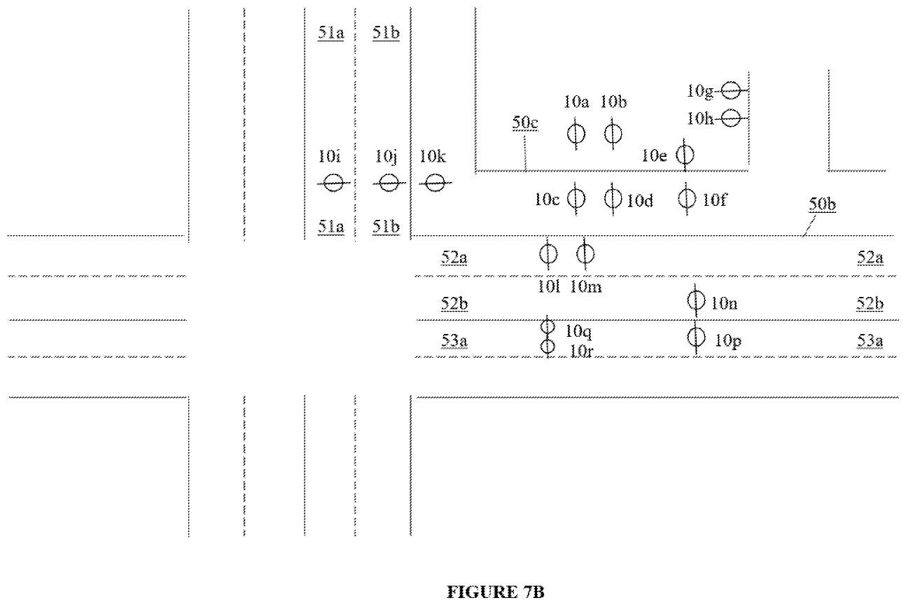

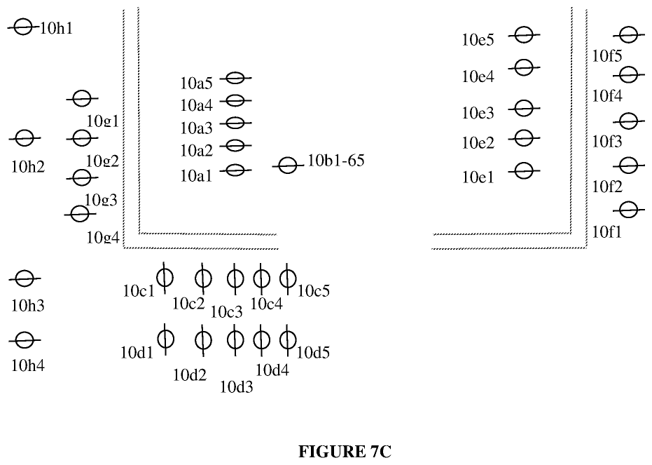

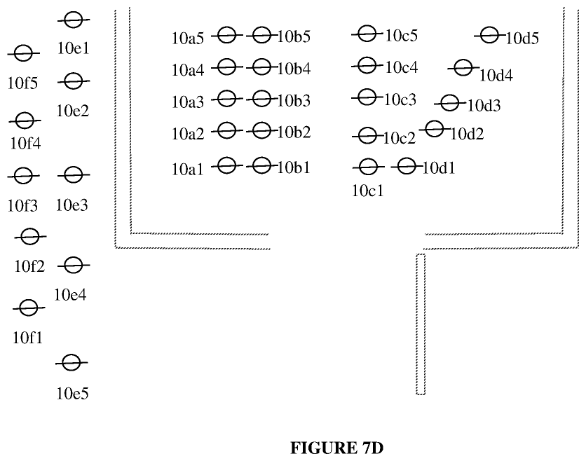

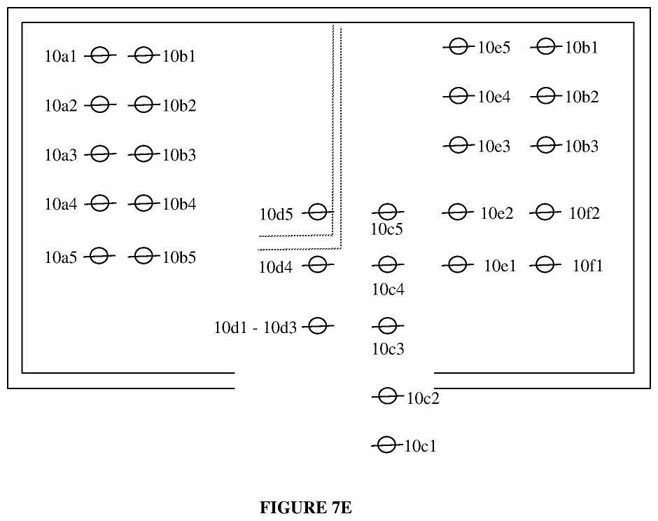

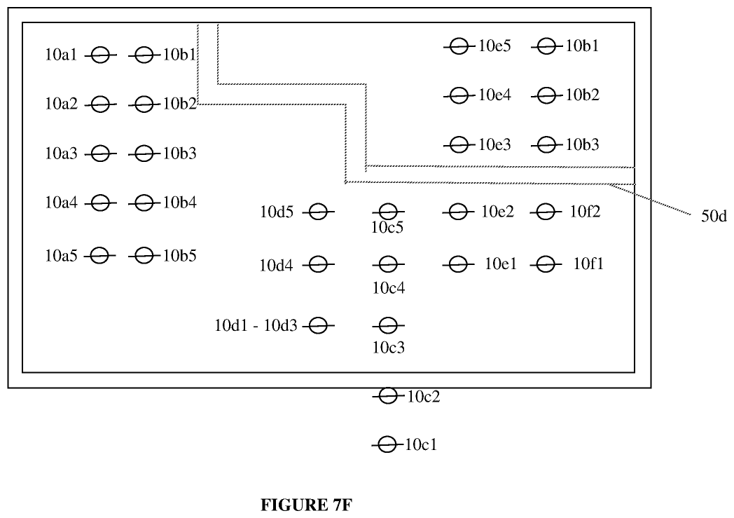

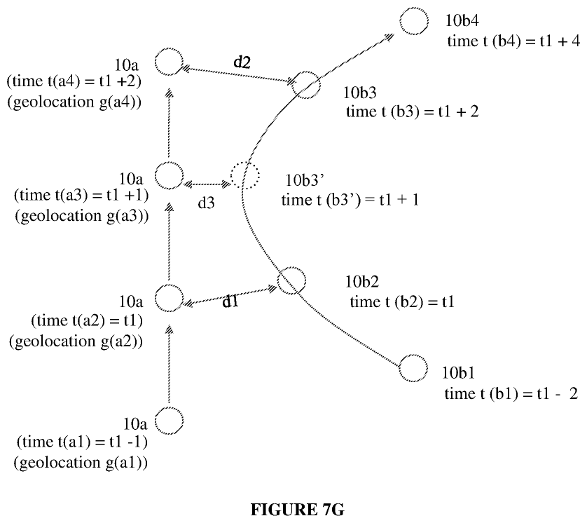

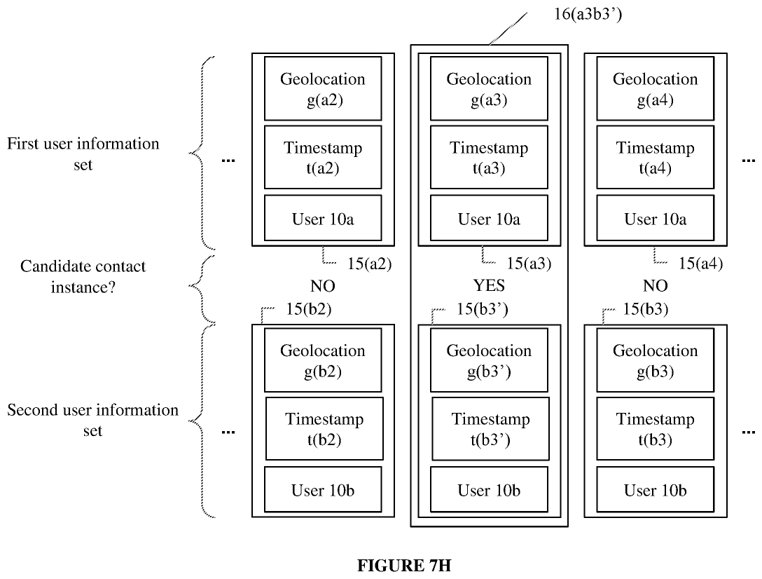

The processor 200 may receive information sets 15 from each user 10 and/or user device 10 (and/or particular location information sets 15′ from other devices 10) periodically, intermittently, continuously, after meeting certain conditions, etc. For example, the processor 200 may receive, every 1 or 2 seconds, information sets 15 that have been generated by each user device 10 every 1 or 2 seconds. As another example, the processor 200 may receive an accumulation of information sets 15 (e.g., 60 information sets generated by the user device 10 in 1 minute) for each user 10 every 1 minute. As another example, the processor 200 may receive an accumulation of information sets 15 (e.g., 1800 information sets generated by the user device 10 in 1 hour) for each user 10 every 1 hour. As another example, the processor 200 may receive an accumulation of information sets 15 for each user 10 each time the user device 10 has access to a WiFi network. As another example, the processor 200 may receive information sets 15 (e.g., information sets generated every 5 seconds) for each user 10 when the user device 10 detects a movement (and/or non-movement) of the user 10 and/or user device 10. Examples of other frequencies for generating the information sets 15 (e.g., when to record/obtain geolocation 11 of the user 10 and/or user device 10) and when information sets 15 should be sent to (or retrieved by) the processor 200 are also contemplated without departing from the teachings of the present disclosure. In example embodiments where a user device 10 generates information sets 15 periodically (e.g., there is a time interval between consecutive or sequentially generated information sets 15 generated by the user device 10; there is a time difference between the timestamps 12 of consecutive or sequentially generated first information sets 15, such as 5 seconds), the processor 200 may be configurable or configured to generate, for any two consecutively or sequentially generated information sets 15 (e.g., two consecutively generated information sets 15 that are consecutively generated by the user device 10 and separated by 4 seconds), one or more processor-generated interpolated, estimated, approximated, and/or predicted information sets 15 (each referred to herein as an “interpolated information set” 15, or the like). For example, the processor 200 may generate an interpolated information set 15 that has an interpolated timestamp 12 selected to be 2 seconds after a first consecutive information set 15 and 2 seconds before a second consecutive information set 15; and the interpolated information set 15 may also have an interpolated geolocation 11 that has been generated based on one or more of the following: the interpolated timestamp 12, a geolocation 11 of the first consecutive information set 15, a geolocation 11 of the second consecutive information set 15, a geolocation 11 of one or more information sets 15 before the first consecutive information set 15, a geolocation 11 of one or more information sets after the second consecutive information set 15, and/or an analysis of historic information sets 15 of the first user 10 (e.g., historic geolocations 11 of the first user 10). Put differently, an interpolated information set 15 is an information set 15 that is not generated by and/or received from a user device 10, but generated by the processor 200 based on an interpolation (and/or estimation and/or approximation and/or prediction) of two consecutive or sequentially generated information sets 15 generated by a user device 10 (and/or an analysis of historic information sets 15 of the first user 10). Each interpolated information set 15 will have an interpolated timestamp 12 that has been interpolated and/or selected based on the timestamps 12 of such two consecutive or sequentially generated information sets 15 (e.g., if the timestamps 12 of the two consecutive or sequentially generated information sets 15 is 22:09:00 and 22:09:04, or a time interval of 4 seconds, the timestamp 12 of the interpolated information set 15 may be 22:09:02). Each interpolated information set 15 will also have an interpolated geolocation 11 that has been interpolated and/or selected based on one or more of the following: the interpolated timestamp 12 (e.g., relative to the timestamps 12 of the two consecutive information sets 15), the geolocations 11 of the two consecutive or sequentially generated information sets 15, geolocations 11 of previous and/or subsequent information sets 15 before and/or after one or both of the two consecutive or sequentially generated information sets 15, geolocations 11 of other interpolated information sets before and/or after the interpolated information set 15, geolocations 11 of other interpolated information sets before and/or after the two consecutive or sequentially generated information sets 15, and/or an analysis of historic information sets 15 of the first user 10 (e.g., from a database 30 of historic information sets 15 and/or historic geolocations 15) (e.g., if a distance between the two consecutive or sequentially generated information sets 15 is 1.6 meters, then a distance between the geolocation 11 of the interpolated information set 15 and any of the two consecutive or sequentially generated information sets 15 will be 0.80 meters (or at a midpoint position along a line drawn between the geolocations 11 of the two consecutive or sequentially generated information sets 15)). For example, as illustrated in FIG. 7G and FIG. 7H, a user 10b has generated the following information sets 15: an information set 15 with geolocation g(b1) and timestamp t(b1); an information set 15 with geolocation g(b2) and timestamp t(b2); an information set 15 with geolocation g(b3) and timestamp t(b3); and an information set 15 with geolocation g(b4) and timestamp t(b4). As each information set 15 is generated 2 seconds apart from one another, the processor 200 is configurable or configured to generate an interpolated information set 15(b3′) having interpolated timestamp t(b3′) and interpolated geolocation g(b3′), where the interpolated timestamp t(b3′) is 1 second after timestamp t(b2) of information set 15(b2) and 1 second before timestamp t(b3) of information set 15(b3), and where the interpolated geolocation g(b3′) has been interpolated based on at least the interpolated timestamp t(b3′), the geolocation g(b2) of information set 15(b2), the geolocation g(b1), the geolocation g(b3) of information set 15(b3), and the geolocation g(b4). It is to be understood that references to an information set 15, user information set 15, candidate information set 15, or the like, in the present disclosure may also include interpolated information sets 15 without departing from the teachings of the present disclosure.

In example embodiments, the processor 200 may be configurable or configured to receive, from a user 10 (or a particular user 10 or an administrator 40 or a trusted authority 40), a special information set 15 that has been selectively generated by the user 10 (or the particular user 10 or the administrator 40 or the trusted authority 40, respectively) (referred to herein as a “special information set” 15). Similar to a normal information set 15, a special information set 15 also includes a geolocation 11 of the user 10 and a timestamp 12 of the geolocation of the user 10. However, the processor 200 may distinguish between a special information set 15 and a normal information set 15 in one or more ways. For example, each special information set 15 may include one or more of the following aspects used to indicate that the information set 15 is a special information set 15: a particular value in a status field (e.g., a “00”, or the like, indicates a normal information set 15; a “01”, or the like, indicates a special information set 15 sent from a user 10; a “10”, or the like, indicates a special information set 15 sent from a particular user 10; a “11”, or the like, indicates a special information set 15 sent from an administrator 40, trusted authority 40, or others); a special character or series of characters; a mark or tag; etc. The user 10 may generate a special information set 15 by performing a dedicated action or sequence of actions, such as: pressing a dedicated button, link, or the like on a mobile application on the user device 10 (or any other action or actions, such as a swipe, long press, multi-touch, gesture, voice command, facial expression, etc.); pressing a dedicated emergency button on the lock screen of the user device 10; etc. Special information sets 15 may be generated so as to request or instruct the processor 200 to perform a real-time, near real-time, on-demand, prioritized, immediate, and/or out-of-turn/skip-the-line processing of a risk or likelihood that the user 10 (based on the geolocation 11 and/or timestamp 12 in the special information set 15) is physically exposed and/or being physically exposed to a particular user 10, potential particular user 10, particular location 11′, and/or potential particular location 11′. For example, in a situation where the user 10 (e.g., a second user 10) is in a busy restaurant and the second user 10 notices one or more other users 10 nearby (e.g., a first user 10) who appears to be a potential particular user 10 or a particular user 10 (e.g., sneezing, coughing, and/or other noticeable symptoms; and/or wearing a wristband, etc. indicative of someone who may have tested positive for an infectious/contagious disease and/or someone currently under a restrictive movement order such as a stay-at-home order, quarantine order, isolation order, etc.), the second user 10 may selectively generate such special information set 15 so as to notify the processor 200 that this particular geolocation 11 and timestamp 12 (i.e., in the special information set 15) should be processed right away or as soon as possible (and/or marked/tagged as a potential particular location 11′). Upon receiving the special information set 15, the processor 200 is configurable or configured to perform one or more of the actions described in the present disclosure. For example, the processor 200 may perform searches for recently received information sets 15 of other users 10 nearby (e.g., other users 10 within a certain radius or distance of the geolocation of the second user 10, other users 10 in the restaurant, etc.; referred to as “nearby users” 10). For such recently received information sets 15, the processor 200 may perform one or more actions described in the present disclosure (e.g., generate candidate contact instances, generate inaccuracy score, generate non-contact score, derive risks/likelihoods, etc.) and/or one or more of the following: mark/tag all such nearby users 10 (and/or mark/tag information sets 15 recently and/or being received by such nearby users 10) in the area (or within a certain radius or distance, in the restaurant, etc.) as being potentially exposed to a potential particular location 11′ (e.g., amending information sets 15 from nearby users 10 to be a special information set 15, or the like); determine whether any such nearby users 10 have already been identified as particular users 10 (and/or someone who may be under a restrictive movement order such as a stay-at-home order); perform processing to identify another set of other users 10 who have previously been in contact with such nearby users 10 (e.g., generate candidate contact instances 16, perform one or more of the analyses described in the present disclosure, etc.) to see if such set of other users 10 have been identified as particular users 10 (and/or someone who may be under a restrictive movement order such as a stay-at-home order); and so on. In performing such searches and processing, the processor 200 may identify the second user 10 and/or other nearby users 10 in the area as either being particular users 10 and/or potential particular users 10 (based on whether such nearby users 10 have been in contact with particular users 10, whether such nearby users 10 have been in contact with other users 10 who have been in contact with particular users 10, whether such nearby users 10 have been in contact with other users 10 who have been in contact with other users 10 who have been in contact with particular users 10, and so on). It is recognized in the present disclosure that enabling users 10 to perform such proactive, real-time, and/or on-demand requesting, alerting, and/or checking via special information sets 15 may enable, allow for, and/or assist in real-time and/or near real-time contact tracing (and/or preventive measures to alert users 10 to move away from nearby users 10 and/or the location). Such a feature may also identify users 10 of interest who have been given self-quarantine orders, stay at home orders, other restrictive movement orders, etc. In situations where the processor 200 has identified a particular user 10, users 10 of interest, and/or potential particular users 10 pursuant to the second user 10 selectively generating special information sets 15, one or more actions may be taken. For example, an emergency or urgent notification may be sent to an administrator 40, trusted authority 40, law enforcement authorities 40, health authorities 40, and/or others (e.g., the second user 10, nearby users 10, the restaurant, etc.). In the case of administrators 40 and/or trusted authorities 40 receiving such notifications, one or more actions may be taken (e.g., dispatch law enforcement and/or health authorities to quarantine, shut down, monitor, etc. the particular location 11′ and/or users 10 in the area; alerting the restaurant; etc.). It is to be understood that references to an information set 15, user information set 15, or the like, in the present disclosure may also include special information sets 15 without departing from the teachings of the present disclosure.

After one or more information sets 15 from a plurality of users 10 and/or user devices 10 are received by the processor 200, the processor 200 is configurable or configured to determine whether any users 10 and/or user devices 10 may have been in “contact” with other users 10 and/or user devices 10 (e.g., within a certain distance of one another; the processor 200 may also determine whether there may have been contact within a certain period of time, such as the last 14 days and/or between certain dates/times). The processor 200 determines whether there may have been such contact by processing the information sets 15 so as to generate one or more candidate contact instances 16. An example candidate contact instance 16 is illustrated in FIG. 1D. As described in the present disclosure, a candidate contact instance 16 includes an information set 15 of a first user 10 (e.g., a first candidate first user information set 15; where the first user 10 may be a particular user 10 (e.g., an infected and/or contagious person who has been identified by an administrator 40 or trusted authority 40)) and an information set 15 of a second user 10 (e.g., a first candidate second user information set 15 or a first candidate first other user information set 15). In an example embodiment, the candidate contact instance 16 is generated based on at least the following conditions: a distance between a geolocation 11 in the first candidate first user information set 15 and a geolocation 11 in the first candidate second user information set 15 is less than a threshold distance value (e.g., 0.5 meters, 0.75 meters, 1 meter, 2 meters, etc.) (referred to herein as the “minimum distance requirement”); and an amount of time between a timestamp 12 in the first candidate first user information set 15 and a timestamp 12 in the first candidate second user information set 15 is less than a threshold time value (e.g., 0.5 seconds, 1 second, 2 seconds, etc.) (referred to herein as the “minimum time requirement”). In situations where the processor 200 receives a request from a requestor (e.g., administrator 40, trusted authority 40, or the like) to perform contact tracing for a particular user 10 (e.g., a first user 10), the processor 200 is configurable or configured to search for information sets 15 of the first user 10 (or first user information sets 15) matching one or more criterion provided by the requestor. For example, if the requestor provides a date range of “the last 14 days”, then the processor 200 will obtain some or all first user information sets 15 for the last 14 days (or obtained first user information sets 15). The processor 200 will then search for information sets 15 of one or more other users 10 (or other user information sets 15) based on the geolocations 11 and timestamps 12 of the obtained first user information sets 15 (and/or the requestor's criterion (e.g., the last 14 days)) to determine whether any of the other user information sets 15 and any of the obtained first user information sets 15 meet the minimum distance requirement and minimum time requirement. Those other user information sets 15 and first user information sets 15 that meet the minimum distance requirement and minimum time requirement are then used to generate candidate contact instances 16. Alternatively or in addition, the processor 200 may be configurable or configured to continuously, periodically, and/or intermittently (e.g., during non-peak periods when the processor 200 receives less information sets 15, such as between midnight until 6 am, etc.) perform processing of some or all received information sets 15 (e.g., without receiving a request for contact tracing from a requestor) so as to identify information sets 15 from users 10 that meet the minimum distance requirement and minimum time requirement, and proactively generate candidate contact instances 16 accordingly. Such proactively generated candidate contact instances 16 may then be stored and ready for if and when a requestor submits a request for contact tracing, in which case the processor 200 may first search (or search in parallel) a database 30 of proactively generated candidate contact instances 16 (which is recognized in the present disclosure to save time and/or processing loads, especially during peak or high processing times). For the above example, the processor 200 may first search the database 30 for any proactively generated candidate contact instances 16 having information sets 15 for the particular user 10 (the first user 10), and if found, those proactively generated candidate contact instances 16 are then identified and provided to one or more elements of the processor 200 (e.g., the physical exposure assessment processor 230, as described in the present disclosure) for further processing, including deriving of risks/likelihoods of physical exposure, etc.

In example embodiments, user devices 10 may be configurable or configured to generate information sets 15 at a specific (e.g., default) frequency, required frequency (e.g., standardized), and/or desired frequency (e.g., user-selectable). It is recognized in the present disclosure that when user devices 10 generate information sets 15 at higher frequencies (e.g., less time between generating information sets 15) higher power consumption, higher processing power demands, higher memory storage (e.g., if information sets 15 are saved on the user device 10 and sent at a later time), and/or higher data communication requirements (e.g., consuming data in mobile phone data communication plans) may arise as a result. However, it is also recognized in the present disclosure that lowering the frequency of generating information sets 15 (e.g., generating information sets 15 every 8 seconds as compared to every 2 seconds) may result in the processor 200 possibly missing potentially relevant contact instances, such as in situations where a first user 10 and second user 10 are within the minimum distance requirement and minimum time requirement at a time t1 but the first user device 10 (first user 10) and/or the second user device 10 (second user 10) were not scheduled to generate an information set 15 at that particular time (t1). In an example embodiment, the processor 200 is configurable or configured to generate one or more interpolated information sets 15 that each have an interpolated timestamp 12 and an interpolated geolocation 11. For example, as illustrated in FIGS. 7G and 7H, a first user 10a may generate the following information sets 15: an information set with geolocation g(a1) and timestamp t(a1); an information set 15(a2) with geolocation g(a2) and timestamp t(a2); an information set 15(a3) with geolocation g(a3) and timestamp t(a3); and an information set 15(a4) with geolocation g(a4) and timestamp t(a4). Furthermore, a second user 10b may generate the following information sets 15: an information set with geolocation g(b1) and timestamp t(b1); an information set 15(b2) with geolocation g(b2) and timestamp t(b2); an information set 15(b3) with geolocation g(b3) and timestamp t(b3); and an information set with geolocation g(b4) and timestamp t(b4). The first user device 10 may be configured so as to generate information sets 15 that are 1 second apart from one another, and the second user device 10 may be configured so as to generate information sets 15 that are 2 seconds apart from one another. Without example embodiments of generating interpolated information sets 15, the processor 200 may not generate any candidate contact instances in such a situation, especially if the distance d1 does not meet the minimum distance requirement and the distance d2 does not meet the minimum distance requirement (and if the distance between g(a3) and g(b3) does not meet the minimum distance requirement; and if the distance between g(a3) and g(b2) does not meet the minimum distance requirement). In applying example embodiments of generating interpolated information sets 15, the processor 200 is configurable or configured to generate an interpolated information set 15(b3′) having an interpolated timestamp t(b3′) and an interpolated geolocation g(b3′). In such an example, the interpolated timestamp t(b3′) is 1 second after timestamp t(b2) of information set 15(b2) and 1 second before timestamp t(b3) of information set 15(b3). Furthermore, the interpolated geolocation g(b3′) has been interpolated based on at least the interpolated timestamp t(b3′), the geolocation g(b2) of information set 15(b2), the geolocation g(b1), the geolocation g(b3) of information set 15(b3), and the geolocation g(b4). In generating the interpolated information set 15, the interpolated timestamp t(b3′) for the first user 10a and the timestamp t(a3) for the second user 10b are determined to meet the minimum time requirement. Furthermore, a distance d3 between the interpolated geolocation g(b3′) for the first user 10a and the geolocation g(a3) for the second user 10b are determined to meet the minimum distance requirement. Accordingly, the processor 200 is configurable or configure to generate a candidate contact instance 16(a3b3′) based on and having the information set 15(a3) and the interpolated information set 15(b3′).

In example embodiments, a candidate contact instance 16 may be generated by one or more user devices 10 (instead of the processor 200 generating the candidate contact instance 16). For example, in situations where user devices 10 have Bluetooth enabled (or WiFi or any other form of wireless communications) so as to communicate with other user devices 10, example embodiments may include configuring user devices 10 to receive user information sets 15, or the like, from other user devices 10 (and/or location-based information sets 15′ from devices installed at particular locations 11′) via Bluetooth when the user devices 10 are within communication range (or less, such as in example embodiments where Bluetooth signal strength and/or geolocation are used to assess when (or within what distance) user information sets 15 should be exchanged). Once received, the user device 10 may then generate candidate contact instance(s) 16 (e.g., using the user information set(s) 15 from the other user device 10 and user information set(s) 15 generated by the user device 10) or send information sets 15 of both user devices 10 to the processor 200 for the processor 200 to generate the candidate contact instances 16). In example embodiments, one user device 10 may send information sets 15 for both user devices 10, or both user devices 10 may send information sets 15 for both user devices 10. In addition to or alternatively, example embodiments may configure user devices 10 to send/transmit/exchange user information sets 15 to or with other devices 10 (and/or location-based information sets 15′ from devices installed at particular locations 11′) via Bluetooth (or WiFi or any other form of wireless communications) so as to generate candidate contact instances 16 (either generate the candidate contact instances 16 by the other device 10 or have the other user device send information to the processor 200 for the processor 200 to generate the candidate contact instances 16).

It is recognized in the present disclosure that a determination, by the processor 200, that a first user device 10 and a second user device 10 are within a threshold distance of one another at a particular time (i.e., via the minimum distance requirement and minimum time requirement used to form a candidate contact instance 16), may not be adequate, sufficient, accurate, and/or complete enough to determine a risk or likelihood that the first and second users 10 were physically exposed to one another. As used in the present disclosure, a reference to a second user 10 being “physically exposed”, or the like, to a first user 10, or vice versa, may include, but is not limited to, situations where the first and second users 10 are within a certain distance of one another and there are no physical barriers, or the like, between the first and second users 10 to prevent (or block or inhibit or reduce a likelihood of) a contagion, virus, bacteria, radiation, infectious and/or contagious disease, or the like, from passing, traveling, carrying, flying, or the like, from the first user 10 to the second user 10, or vice versa. As described in the present disclosure, first and second user devices 10 may meet the minimum distance requirement and minimum time requirement but not be (or have a lower, reduced, or low risk or likelihood of being) physically exposed to one another. Examples of such situations include, but are not limited to, first and second users 10 meeting the minimum distance requirement and minimum time requirement but the first and second users 10 were separated by a wall, window, another user, vehicle door (e.g., a first user 10 is inside a vehicle and a second user 10 is on a sidewalk), etc. As another example situation, the geolocations 11 of the first and second users 10 may meet the minimum distance requirement and minimum time requirement but the first and second users 10 were on different levels/floors of a multi-level building (i.e., the first and second users 10 were separated by a floor).

After generating/receiving each candidate contact instance 16 (e.g., those generated by the processor 200, user devices 10, and/or other devices 10; and either generated via a request from a requestor or proactively generated), the processor 200 is configurable or configured to derive or assess a likelihood or risk that a user 10 (e.g., the second user 10) was physically exposed to another user 10 (e.g., the first user 10) at, for, based on, and/or during the candidate contact instance 16. The processor 200 may perform this by deriving an inaccuracy score and/or a non-contact score for each candidate contact instance 16. As described in the present disclosure, the inaccuracy score for each candidate contact instance 16 represents a likelihood that the candidate contact instance 16 is inaccurate and/or incorrect; and the non-contact score for each candidate contact instance 16 represents a likelihood that the second user 10 was not physically exposed to the first user during the candidate contact instance 16 (alternatively or in addition, a contact score can also be derived for each candidate contact instance 16, which represents a likelihood that the second user 10 was physically exposed to the first user 10 during the candidate contact instance 16; for example, if the non-contact score is in the form of a percentage probability of not being physically exposed (e.g., 25%), then the contact score may be a percentage probability of being physically exposed (e.g., 75%, or 100%-25%)). As described in the present disclosure, the likelihood that the second user 10 was physically exposed to the first user 10 during the candidate contact instance 16 may be based on the non-contact score (and/or the contact score); and the non-contact score may be derived based on one or more of the following: a layout analysis (e.g., an analysis, based on one or more floor layouts, of a likelihood of a presence of a physical barrier (e.g., a wall, window, door, screen, floor, etc.), or the like, between the first user 10 and the second user 10 during the candidate contact instance 15); map analysis (e.g., an analysis, based on one or more maps, of a likelihood of a presence of a physical barrier (e.g., a wall, window, door, screen, floor, etc.), or the like, between the first user 10 and the second user 10 during the candidate contact instance 15); a first transportation-based separation analysis (e.g., an analysis, based on one or more maps, of a likelihood that the first user 10 was in a vehicle and/or the second user 10 was in a vehicle during the candidate contact instance 15); a second transportation-based separation analysis (e.g., an analysis, based on travel speeds of the first user 10 and the second user 10, of a likelihood that the first user 10 and the second user 10 were not traveling using the same transport mode during the candidate contact instance 15); a third transportation-based separation analysis (e.g., an analysis, based on travel directions of the first user 10 and the second user 10, of a likelihood that the first user 10 was in a vehicle and/or the second user 10 was in a vehicle during the candidate contact instance 15); and/or a different level analysis (e.g., an analysis, based on one or more floor layouts and/or maps, of a likelihood that the first user 10 and the second user 10 were not on the same levels/floors of a multi-level building during the candidate contact instance 15).

After deriving the non-contact score for a candidate contact instance 15, the processor 200 is configurable or configured to identify, for the candidate contact instance 15, the second user 10 as having a risk of being physically exposed to the first user 10 when the non-contact score is equal to or below a threshold non-contact score (e.g., if the threshold non-contact score is 50% chance of not being physically exposed and the non-contact score is 20% chance of not being physically exposed (which is below the threshold non-contact score), then the second user 10 will have a high risk (i.e., 80% chance) of having been physically exposed to the first user 10).

For candidate contact instances 15 in which the processor 200 identifies the second user 10 as having a high risk of being physically exposed to the first user 10 (e.g., when the non-contact score for the candidate contact instance 15 for the second user 10 and the first user 10 is equal to or below a threshold non-contact score), the processor 200 is configurable or configured to perform, among other things, one or more of the following: send a notification to an administrator 40 and/or trusted authority 40 (e.g., government entity); send a notification to the second user 10 to inform the second user 10 that he/she has a high risk of being physically exposed to a particular user 10 (the identity of the first user 10 may or may not be disclosed; in preferred embodiments, the identity of the first user 10 is not disclosed and/or remains anonymized from the perspective of the second user 10); send a notification to the first user 10 to inform the first user 10 that he/she has a high risk of physically exposing to another user 10 (the identity of the second user 10 may or may not be disclosed; in preferred embodiments, the identity of the second user 10 is not disclosed and/or remains anonymized from the perspective of the first user 10); and/or generate particular location information set 15′ for particular locations 11′ (e.g., address, building name, landmark, business name, set of geolocations, etc.) matching and/or including the geolocations 11 of information set 15 for the first user 10 (e.g., in the past 14 days) and geolocations 11 of information sets 15 for the second user 10 (e.g., starting from the timestamp 12 (of the information set 15 for the second user 10) of the candidate contact instance 15 until present). It is recognized in the present disclosure that in applications/situations where a particular country requires all persons physically within the country to install a mobile application that generates and sends information sets 15 (and/or candidate contact instances 16, such as via Bluetooth) as described in the present disclosure, performing contact tracing and identifying and/or informing users 10 who have a high risk of being physically exposed to a known particular user 10 will be highly effective. It is recognized in the present disclosure that even in applications/situations where a particular country does not require all persons physically within the country to install such a mobile application that generates and sends information sets 15 (and/or candidate contact instances 16, such as via Bluetooth) as described in the present disclosure, effective contact tracing and identifying and/or informing users 10 having a high risk of having been physically exposed to a known particular user 10 can still be achieved. For example, in addition to or in replacement of using thermal scanners, or the like, to check temperatures of people before entering a premise (e.g., retail shop, shopping mall, office building, office complex, sports facility, public transportation vehicle or facility, taxi or ride-hail vehicle, etc.), such premises may require users 10 to show a “clear” status, “green” status, or the like, from such mobile applications before being permitted to enter. In this regard, users 10 may be required to have installed/used the mobile application for a certain period of time (e.g., for at least the past 10 days); and those users 10 who have received a notification, from the processor 200, of having a high risk of being physically exposed to a particular user 10, as described above and in the present disclosure, will have an indication on the mobile application (e.g., “not clear” status, “orange” or “red” status, or the like) and not be permitted to enter such premises until they follow certain instructions (e.g., visit a medical facility, administrator 40, trusted authority 40, or the like, to clear the notification, etc.). Alternatively or in addition, certain employers, groups, conferences, events, etc. may also require employees, members, attendees, etc. to install and use such mobile applications in such manner.

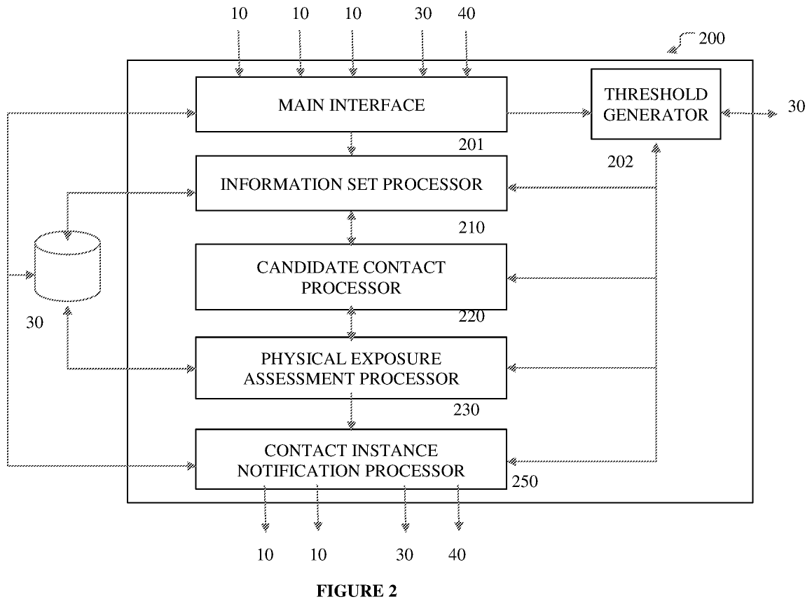

To perform the processes and/or methods described above and in the present disclosure, example embodiments of the processor 200 include one or more elements. For example, as illustrated in FIG. 2, the processor 200 includes one or more main interfaces 201. The processor 200 also includes one or more information set processors 210. The processor 200 also includes one or more candidate contact processors 220. The processor 200 also includes one or more physical exposure assessment processors 230. The processor 200 also includes one or more contact instance notification processors 240. Although the figures illustrate one main interface 201, one information set processor 210, one candidate contact processor 220, one physical exposure assessment processor 230, and one contact instance notification processor 240, it is to be understood in the present disclosure that the processor 200 may include more or less than one main interfaces 201, more or less than one information set processors 210, more or less than one candidate contact processors 220, more or less than one physical exposure assessment processors 230, and more or less than one contact instance notification processors 240 without departing from the teachings of the present disclosure. It is also to be understood in the present disclosure that, although the functions and/or processes performed by the processor 200 are described in the present disclosure as being performed by particular elements of the processor 200, the functions and/or processes performed by a particular element of the processor 200 may also be performed by one or more other elements and/or cooperatively performed by more than one element of the processor 200 without departing from the teachings of the present disclosure. It is also to be understood that, although the functions and/or processes performed by the processor 200 are described in the present disclosure as being performed by particular elements of the processor 200, the functions and/or processes performed by two or more particular elements of the processor 200 may be combined and performed by one element of the processor 200 without departing from the teachings of the present disclosure.

These elements of the processor 200 will now be further described with reference to the accompanying figures.

The Main Interface (e.g., Element 201).

As illustrated in FIG. 2, an example embodiment of the processor 200 includes one or more main interfaces (e.g., main interface 201). The main interface 201 is configurable or configured to act as a communication interface for the processor 200. For example, the main interface 201 is configurable or configured to receive user information sets 15 from users 10, location-based information sets 15′ from designated devices (e.g., installed at particular locations 11′), candidate contact instances 16 (e.g., when generated by user devices and/or other processors 200), information from databases 30, and information from one or more other elements of the processor 200 and/or other processors 200. The main interface 201 (and/or the contact instance notification processor 250) is also configurable or configured to send information and/or notifications to users 10, designated devices installed at particular locations 11′, the network 20, databases 30, and/or administrators 40 and/or trusted authorities 40.

After the main interface 201 receives the above information, the main interface 201 is configurable or configured to selectively provide information to one or more other elements of the processor 200 and/or other processors 200 for further processing.

The Information Set Processor (e.g., Element 210).

As illustrated in FIG. 2, an example embodiment of the processor 200 includes one or more information set processors (e.g., information set processors 210). The information set processor 210 is configurable or configured to receive information sets 15 from a plurality of users 10 and/or user devices 10 (e.g., via the main interface 201). In this regard, user devices 10 may be configurable or configured to generate information sets 15 and transmit such information sets 15 to the main interface 201. Alternatively or in addition, the information set processor 210 is configurable or configured to receive geolocation 11 and temporal 12 information from a plurality of users 10 and/or user devices 10, and generate user information sets 15 for each of the users 10 and/or user devices 10 based on such geolocation 11 and temporal 12 information. As described in the present disclosure, the information set processor 210 is also configurable or configured to receive special information sets 15 from user devices 10 (and perform prioritized, immediate, and/or out-of-turn/skip-the-line processing of such special information sets 15), receive location-based information sets 15 (e.g., particular location information sets 15), and also generate interpolated information sets 15.

As illustrated in FIG. 1B, each information set 15 for a user 10 and/or user device 10 includes a geolocation 11 of the user 10 and/or user device 10 (and/or location 11 relative to a geolocation of a previous information set 15 for the user 10; location 11 relative to a reference location; location 11 relative to a geolocation of another user 10; etc.) and a corresponding timestamp 12 for the geolocation 11 of the user 10 and/or user device 10 (and/or time 12 relative to a timestamp of a previous or subsequent information set 15 for the user 10; time 12 relative to a reference time or timestamp; time 12 relative to a timestamp of an information set of another user 10; etc.). Each information set 15 also includes a unique identifier 13 of the user 10 and/or user device 10. Such unique identifier 13 of the user 10 and/or user device 10 may include, but is not limited to, one or more of the following anonymized and/or non-anonymized information 13 of and/or pertaining to the user 10 and/or user device 10: any form of personal identifiable information (PII) 13 of the user 10; legal name 13 or identity 13 of the user 10; a unique user ID 13; a unique user login name 13; a government-issued identification number 13 of the user 10 (e.g., national identification number 13, citizenship number 13, driver license number 13, passport number 13, etc.); a mobile phone number 13 for the user device 10; a device ID number for the user device 10; an email address 13 of the user 10; a unique social media name or handle of the user 10; other personal information 13 of the user 10 (e.g., date of birth, city/country of birth, citizenship, etc.); a residential address 13 for the user 10; employment status 13, information 13, or history 13 of the user 10; family members 13; marital status 13 of the user 10; household members 13 of the user 10; unique series of characters 13 assigned to the user 10; one or more forms of biometrics 13 of the user 10, such as fingerprint scan, retina scan, facial scan, partial facial scan (e.g., with mask); any one or combination of information other than PII that is unique to a user 10; etc.

In an example embodiment, the unique identifier 13 for the user 10 and/or user device 10 may be anonymized by hashing (e.g., via any hashing algorithm including, but not limited to, SHA-256, SHA-3, etc.) one or more of the above information 13 of the user 10 and/or user device 10. Alternatively or in addition, the unique identifier 13 for the user 10 and/or user device may be anonymized in other ways, including encryption, digital signatures, etc., and/or stored in a DLT (e.g., blockchain). In this regard, the user 10, user device 10, and/or mobile application installed on the user device 10 may be configurable or configured to generate such anonymized unique identifier 13 and also de-anonymize such anonymized unique identifier 13 (i.e., to uncover information of the user 10 and/or user device 10). Alternatively or in addition, the processor 200, an administrator 40, and/or a trusted authority 40 may have the private key (or decryption key, etc.) and/or access to a system/processor for de-anonymizing the anonymized unique identifier 13 (e.g., in emergency situations, such as widespread, quickly spreading viruses, global pandemics, etc.). In example embodiments, a mobile application, or the like, is installed on user devices 10 to generate and send information sets 15 for the user 10 and/or user device 10 and receive notifications from the processor 200. For such mobile applications, the anonymized unique identifier 13 for the user 10 and/or user device 10 may be stored in, embedded in, linked to, associated with, and/or used by the mobile application to enable anonymous communications with the processor 200 (e.g., the processor 200 only knows that it is communicating with an anonymized unique identifier 13, but has no knowledge of, access of, and/or ability to derive the identity and/or personal information (e.g., personal identifiable information, or PII) of the user 10 and/or user device 10; communications include receiving generated information sets 15, receiving candidate contact instances 16 (if generated by the user 10 and/or user device 10), and sending notifications of risks of physical exposure, etc.). As privacy and strong protections of personal information are becoming increasingly important around the world, it is recognized in the present disclosure that anonymization of information should be maximized and the types of information obtained (or collected, pulled, received, secured, retained, etc.) from users 10 and/or user devices 10 should be kept at a minimum regardless of whether or not example embodiments of managing contact information are applied as an opt-in service (e.g., mobile application), required service (e.g., in order to use or enter into certain facilities and/or have certain rights), or mandatory service (e.g., required by all citizens, residents, visitors, members, and/or employees). In this regard, although example embodiments described herein are directed to collecting only geolocation, temporal, and unique identification/identifier 13 of each user 10, it is recognized in the present disclosure that other information for each user 10 may also be collected (and one or more such other/additional information may correspondingly be anonymized, such as via linking and/or associating to an anonymized unique identifier 13 of the user 10 and/or user device 10 instead of any personal information of the user 10) and included in a user information set 15 without departing from the teachings of the present disclosure. For example, information generated and/or derivable by the user device 10 from readings/measurements of accelerometers, or the like, pertaining to user movements, steps, and/or speed (and/or instantaneous or average measurement, rate, reading, etc.) may be included for each geolocation 11 in the user information set 15. As another example, information from other applications, such as ride-hailing application, public transportation application, air travel application, train travel application, boat/cruise ship application, etc. may also be used to identify whether the user 10 is in a transportation vehicle and/or to identify other users 10 (e.g., driver(s) 10, workers, and/or other passengers 10) who may be in contact with and possibly being physically exposed to the user 10. In this regard, such information may be used in example embodiments of the non-contact processor 240 to generate non-contact scores and/or risks/likelihoods of physical exposure. As another example, information from sports or fitness-related applications (e.g., Fitbit, Apple Health, Nike Run Club, etc.) may also be included to identify an activity being conducted by the user 10 (e.g., stationery, walking, and/or exercising). As another example, information from map applications (e.g., Google Maps, Apple Maps, etc.), social media applications, shopping applications, photo album applications, etc. may also be included in each user information set 15. As used in the present disclosure, a reference to a geolocation 11, timestamp 12, unique identifier 13, and/or information set 15 for a user 10 may also include references to a geolocation 11, timestamp 12, unique identifier 13, and/or information set 15 for a user device 10, and vice versa.

As illustrated in FIG. 1C, the information set processor 210 may also receive and/or generate information sets 15′ for particular locations 11′ (e.g., address, building name, landmark, business name, particular airplane, particular train or train car, particular bus, boat, taxi, or other vehicles, set of geolocations, etc.). For example, in situations where a particular location 11′ is identified as having been contaminated (and/or visited) by one or more particular users 10, viruses, bacteria, other infectious agents or contagions, radiation, etc., an example embodiment of the information set processor 210 is configurable or configured to receive (e.g., from a designated device installed at the particular location 11′) and/or generate one or more particular location information set 15′ and perform location-based contact tracing (i.e., contact tracing of users 10 who have visited the particular location 11′ during a particular date/time range 12′). It is to be understood in the present disclosure that particular location information sets 15′ may also be generated for any location 11′, including particular locations 11′ that have not yet been confirmed as having been contaminated (and/or visited) by one or more particular users 10, viruses, bacteria, other infectious agents or contagions, radiation, etc. (e.g., in situations where the processor 200 is identifying which locations a recently confirmed particular user 10 has visited so as to identify such locations as particular locations, as described in the present disclosure). Referring to FIG. 1C, the particular location information set 15′ for the particular location 11′ may include location-related information including, but not limited to, an address 11′, a building name 11′, a landmark name 11′, a business name 11′, a particular room 11′ or floor/level 11′, a section 11′ of a building or property/compound, a demarcation or boundary 11′, a set of geolocation(s) 11′, etc., which may be used in example embodiments to perform contact tracing of any users 10 who have visited the particular location 11′ within a particular date/time range. The particular location information set 15′ for the particular location 11′ may also include temporal information 12′, including an associated timestamp 12′ (e.g., starting date) and/or date/time range 12′ (e.g., range of dates/times starting on a date/time when the one or more particular users 10 visited the particular location 11′ until a date/time when the particular location 11′ was closed, cleaned, disinfected, etc. (or no end date/time if still ongoing)). The particular location information set 15′ for the particular location 11′ may also include unique identifier(s) 13′ (anonymized and/or non-anonymized) of one or more of the particular users 10 (and/or potential particular users 10) who have visited the particular location 11′ during a relevant date/time range 12′ or specific dates/times 12′.

It is recognized in the present disclosure that such unique identifier information 13 for users 10 in the particular location information set 15′ may be further applied in example embodiments to perform further contact tracing based on geolocation 11 and temporal information 12 of such users 10. For example, based on the particular location information set 15′, the processor 200 may first compare the particular location information 11′ and temporal information 12′ of the particular location information set 15′ with geolocation 11 and temporal information 12 of user information sets 15 of a plurality of users 10 to identify those users 10 who may have a risk of being physically exposed to a virus, bacteria, infectious agent, contagion, radiation, etc. (or “at-risk users” 10). Thereafter, the information set processor 210 may be configurable or configured to amend or supplement each such particular location information set 15′ to include a unique identifier 13 of such at-risk users 10. In addition to or alternatively, as described in the present disclosure, one or more elements of the processor 200 may perform contact tracing using the geolocation and temporal information 12 of such at-risk users 10 to identify other at-risk users 10 who may have a risk of being physically exposed to the at-risk user 10 (e.g., after the at-risk user 10 visited the particular location 11′). And if such other at-risk users 10 are identified by the processor 200 as having a risk of being physically exposed to the at-risk user 10, the processor 200 may perform contact tracing using the geolocation and temporal information 121 of such other at-risk users 10 to identify other further at-risk users 10 who may have a risk of being physically exposed to such other at-risk users 10. And so on. Furthermore, for each at-risk user 10 (including the at risk user 10, the other at-risk user 10, the other further at-risk user 10, and so on), the processor 200 may perform location-based contact tracing to identify particular locations (e.g., address, building name, landmark, business name, set of geolocations, etc.) where such at-risk users 10 have visited (e.g., based on the geolocation and temporal information of such users 10) and perform contact tracing based on such identified particular locations and date/time of visit of the at-risk user 10. And so on. In an example embodiment, the particular location information sets 15′ may be generated by the processor 200, an administrator 40, and/or a trusted authority 40. In some example embodiments, the particular location information sets 15′ may also be generated by users 10 and submitted to the processor 200, administrator 40, and/or trusted authority 40 for verification, confirmation, and/or approval.

The information set processor 210 may receive information sets 15 from each user 10 and/or user device 10 (and/or particular location information sets 15′) in a variety of ways and frequencies, including periodic, intermittent, continuous, after meeting a certain threshold condition, etc. For example, the processor 200 may receive information sets 15 from each user 10 and/or user device 10 every 1 or 2 seconds (or other amount), where each information set 15 includes unique identifier 13 of the user 10, a geolocation 11 of the user 10, and timestamp 12 (or time since last information set 15, time since a certain information set 15, etc.) for the geolocation 11 (e.g., information set 15 for geolocation 11 of the user 10 at 5:15:00 pm (which is the corresponding timestamp 12 for the geolocation 11), another information set 15 for geolocation 11 of the user 10 at 5:15:02 pm (which is the timestamp 12 for the geolocation 11), another information set 15 for geolocation 11 of the user 10 at 5:15:04 pm (which is the timestamp 12 for the geolocation 11), another information set 15 for geolocation 11 of the user 10 at 5:15:06 pm (which is the timestamp 12 for the geolocation 11), and so on). As another example, the processor 200 may receive a plurality of information sets 15 (e.g., 60 information sets) from each user 10 and/or user device 10 every 1 minute (other amount), where each information set 15 includes unique identifier 13 of the user 10, a geolocation 11 of the user 10, and timestamp 12 for the geolocation 11 that is separated by 1 seconds (or other amount) (e.g., information set 15 for geolocation 11 of the user 10 at 12:08:00 pm, another information set 15 for geolocation 11 of the user 10 at 12:08:01 pm, another information set 15 for geolocation 11 of the user 10 at 12:08:02 pm, another information set 15 for geolocation 11 of the user 10 at 12:08:03 pm, and so on). As another example, the processor 200 may receive a plurality of information sets 15 (e.g., 1800 information sets) from each user 10 and/or user device 10 every 1 hour (other amount), where each information set 15 includes unique identifier 13 of the user 10, a geolocation 11 of the user 10, and timestamp 12 for the geolocation 11 that is separated by 2 seconds (or other amount) (e.g., information set 15 for geolocation 11 of the user 10 at 9:22:00 am, another information set 15 for geolocation 11 of the user 10 at 9:22:02 am, another information set 15 for geolocation 11 of the user 10 at 9:22:04 am, another information set 15 for geolocation 11 of the user 10 at 9:22:06 am, and so on). As another example, the processor 200 may receive a plurality of information sets 15 from each user 10 and/or user device 10 each time the user 10 and/or user device 10 has access to a WiFi network, where each information set 15 includes unique identifier 13 of the user 10, geolocation 11 of the user 10, and timestamp 12 for the geolocation 11 that is separated by 1 seconds (or other amount) (e.g., information set 15 for geolocation 11 of the user 10 at 6:02:00 pm, another information set 15 for geolocation 11 at 6:02:01 pm, another information set 15 for geolocation 11 of the user 10 at 6:02:02 pm, another information set 15 for geolocation 11 of the user 10 at 6:02:03 pm, and so on). As another example, the processor 200 may receive a plurality of information sets 15 (e.g., 60 information sets) from each user 10 and/or user device 10 each time the user device 10 detects a movement of the user device 10, where each information set 15 includes unique identifier 13 of the user 10, a geolocation 11 of the user 10, and timestamp 12 for the geolocation 11 that is separated by 0.5 seconds (or other amount) (e.g., information set 15 for geolocation 11 of the user 10 at 5:16:00.5 pm, another information set 15 for geolocation 11 of the user 10 at 5:16:01.0 pm, another information set 15 for geolocation 11 of the user 10 at 5:16:01.05 pm, another information set 15 for geolocation 11 of the user 10 at 5:16:02.0 μm, and so on). Other frequencies for generating information sets 15 (e.g., when to record/obtain geolocation 11 of the user 10 and/or user device 10) and when information sets 15 should be sent to (or retrieved by) the processor 200 are also contemplated without departing from the teachings of the present disclosure.

The information set processor 210 is then configurable or configured to provide the information sets 15, 15′ (including user information sets 15, particular information sets 15′, interpolated information sets 15, and special information sets 15; also referred to as “candidate information sets” 15, 15′) to one or more elements of the processor 200, including the candidate contact processor 220 (e.g., information set selector 222).

The Threshold Generator (e.g., Element 202).

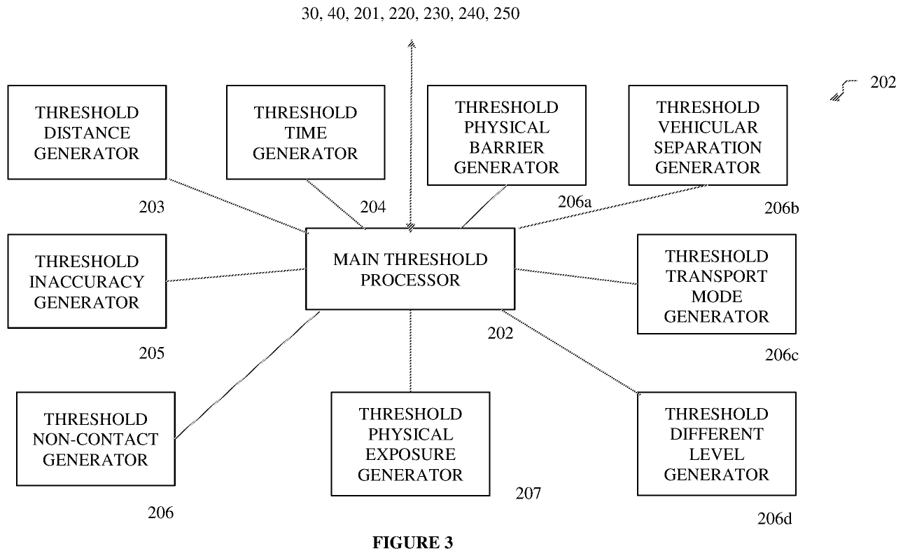

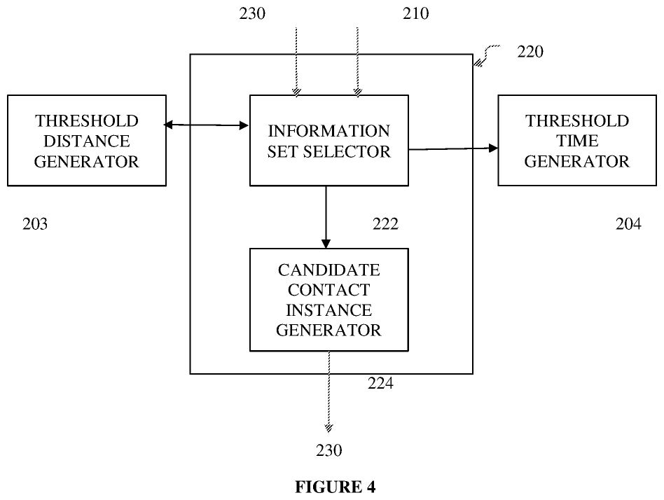

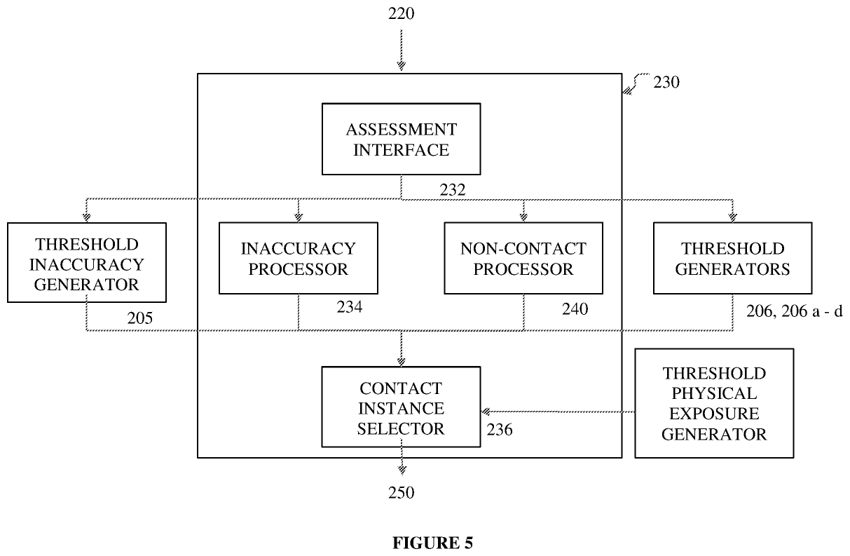

As illustrated in FIG. 2, an example embodiment of the processor 200 includes one or more threshold generators (e.g., threshold generator 202). The threshold generator 202 is configurable or configured to generate threshold values for one or more elements of the processor 200. For example, as illustrated in FIG. 3, the threshold generator 202 may include a main threshold processor 202′. The main threshold processor 202′ may receive information and/or communicate with one or more elements of the system 100 including, but not limited to, databases 30, administrators 40, trusted authorities 40, the main interface 201, the candidate contact processor 220, the physical exposure assessment processor 230, the non-contact processor 240, and/or the contact instance notification processor 250. The threshold generator 202 may also include one or more threshold distance generators 203. As illustrated in FIG. 4, the threshold distance generator 203 may be configurable or configured to generate threshold distance values for the candidate contact processor 220. The threshold generator 202 may also include one or more threshold time generators 204. As illustrated in FIG. 4, the threshold time generator 204 may be configurable or configured to generate threshold time values for the candidate contact processor 220. The threshold generator 202 may also include one or more threshold inaccuracy generators 205. As illustrated in FIG. 5, the threshold inaccuracy generator 205 may be configurable or configured to generate threshold inaccuracy values for the inaccuracy processor 234. The threshold generator 202 may also include one or more threshold non-contact generators 206, one or more threshold physical barrier generators 206a, one or more threshold vehicular separation generators 206b, one or more threshold transport mode generators 206c, and/or one or more threshold different level generators 206d (herein also collectively referred to as the threshold non-contact generators 206, 206a-d). As illustrated in FIG. 5, the threshold non-contact generators 206, 206a-d may be configurable or configured to generate threshold non-contact values (including threshold physical barrier threshold values, threshold vehicular separation values, threshold transport mode values, and/or threshold different level values) for the non-contact processor 240. The threshold generator 202 may also include one or more threshold physical exposure generators 207. As illustrated in FIG. 5, the threshold physical exposure generator 207 may be configurable or configured to generate threshold physical exposure values for the contact instance selector 236.

The threshold generator 202 is configurable or configured to generate one or more threshold values, as described above and in the present disclosure, for and/or in cooperation with one or more other elements of the processor 200.

The Candidate Contact Processor (e.g., Element 220).

As illustrated in FIG. 2, an example embodiment of the processor 200 includes one or more candidate contact processors (e.g., candidate contact processor 220). The candidate contact processor 220 is configurable or configured to receive information sets 15, 15′ (including user information sets 15, particular information sets 15′, interpolated information sets 15, and special information sets 15) from the information set processor 210. The candidate contact processor 220 is then configurable or configured to select one or more information sets 15, 15′. The candidate contact processor 220 is then configurable or configured to generate one or more candidate contact instances 16 based on the information sets 15, 15′ selected by the candidate contact processor 220. After the candidate contact processor 220 generates the candidate contact instances 16, the candidate contact processor 220 provides the candidate contact instances 16 to the physical exposure assessment processor 230 for further processing.

To perform these processes and/or methods, example embodiments of the candidate contact processor 220 include one or more elements. For example, as illustrated in FIG. 4, the candidate contact processor 220 includes one or more information set selectors 222. The candidate contact processor 220 also includes one or more candidate contact instance generators 224. Although the figures may illustrate one information set selector 222 and one candidate contact instance generator 224, it is to be understood in the present disclosure that the candidate contact processor 220 may include more or less than one information set selector 222 and more or less than one candidate contact instance generator 224 without departing from the teachings of the present disclosure. It is also to be understood in the present disclosure that, although the functions and/or processes performed by the candidate contact processor 220 are described in the present disclosure as being performed by particular elements of the candidate contact processor 220, the functions and/or processes performed by a particular element of the candidate contact processor 220 may also be performed by one or more other elements and/or cooperatively performed by more than one element of the processor 200 without departing from the teachings of the present disclosure. It is also to be understood that, although the functions and/or processes performed by the candidate contact processor 220 are described in the present disclosure as being performed by particular elements of the candidate contact processor 220, the functions and/or processes performed by two or more particular elements of the candidate contact processor 220 may be combined and performed by one element of the processor 200 without departing from the teachings of the present disclosure.

These elements of the candidate contact processor 220 will now be further described with reference to the accompanying figures.

The Information Set Selector (e.g., Element 222).

As illustrated in FIG. 4, an example embodiment of the candidate contact processor 220 includes one or more information set selectors (e.g., information set selector 222). The information set selector 222 is configurable or configured to receive candidate information sets 15, 15′ (including candidate user information sets 15, candidate particular information sets 15′, candidate interpolated information sets 15, and candidate special information sets 15) from the information set processor 210. The information set selector 222 is then configurable or configured to select candidate information sets 15, 15′ from among the plurality of candidate information sets 15, 15′ received from the information set processor 210. In an example embodiment, the candidate information sets 15, 15′ selected by the information set selector 222 are those candidate information sets 15, 15′ that satisfy at least the following conditions: a distance between a geolocation 11 in a candidate information set 15 for a first user 10 (e.g., a first candidate first user information set 15) and a geolocation 11 in a candidate information set 15 for a second user 10 (e.g., a first candidate second user information set 15) is less than a threshold distance value; and an amount of time between a timestamp 12 in the first candidate first user information set 15 and a timestamp 12 in the first candidate second user information set 15 is less than a threshold time value. Although example embodiments described in the present disclosure are directed to selecting a candidate information set 15 from one user 10 (e.g., a first user 10) and selecting another candidate information set 15 from another user (e.g., a second user 10) based on the above two conditions (i.e., difference in distance between geolocations 11 are within a threshold distance value, and difference in time between timestamps 12 are within a threshold time value), it is recognized in the present disclosure that the candidate contact processor 220 may also select candidate information sets from more than two different users 10 without departing from the teachings of the present disclosure. For example, the candidate contact processor 220 may select a candidate information set 15 for a first user 10, a candidate information set 15 for a second user 10, and a candidate information set 15 for a third user 10, so long as the three candidate information sets 15 all satisfy the same conditions described above (i.e., difference in distance between geolocations 11 in all three candidate information sets 15 are within a threshold distance value, and difference in time between timestamps 12 in all three candidate information sets 15 are within a threshold time value).

The candidate information sets 15, 15′ selected by the information set selector 222 are then provided to the candidate contact instance generator 224.

The Candidate Contact Instance Generator (e.g., Element 224).

In an example embodiment, the candidate contact processor 220 includes one or more candidate contact instance generators (e.g., candidate contact instance generator 224). The candidate contact instance generator 224 is configurable or configured to receive selected candidate information sets 15, 15′ (including candidate user information sets 15, candidate particular information sets 15′, candidate interpolated information sets 15, and candidate special information sets 15) from the information set selector 222. The candidate contact instance generator 224 is then configurable or configured to generate one or more candidate instances 16. An example contact instance 16 is illustrated in FIG. 1D.

In an example embodiment, the candidate contact instance 16 includes a selected candidate information set 15 of a first user 10 (e.g., a first candidate first user information set 15, which may be the particular user 10 who has been confirmed as having a contagious disease) and a selected candidate information set 15 of a second user 10 (e.g., a first candidate second user information set 15, or a first candidate first other user information set 15). The candidate contact instance generator 224 cooperates with the information set selector 22 to generate each candidate contact instance 16 based on at least the conditions described above and in the present disclosure for the information set selector 222, including: a distance between a geolocation 11 in the first candidate first user information set 15 and a geolocation 11 in the first candidate second user information set 15 is less than a threshold distance value; and an amount of time between a timestamp 12 in the first candidate first user information set 15 and a timestamp 12 in the first candidate second user information set 15 is less than a threshold time value.

It is to be understood in the present disclosure that a candidate contact instance 16 may also be generated by user devices 10 and/or other devices (e.g., designated devices at particular locations 11, 11′). For example, in situations where user devices 10 have Bluetooth (or WiFi or any other form of wireless communications) so as to communicate with other user devices 10 (and/or designated devices), example embodiments may configure user devices 10 (and/or designated devices) to receive user information sets 15 from other user devices 10 (and/or location-based information sets 15′ from devices installed at particular locations 11′) via Bluetooth (or WiFi or any other form of wireless communications) so as to generate candidate contact instances 16 (either generate the candidate contact instances 16 by the user device 10 or send information to the processor 200 for the processor 200 to generate the candidate contact instances 16). For example, if or when a user 10 and another user 10 are in Bluetooth communication range of one another, the user device 10 and the other user device 10 are configured to exchange (or one device sends to another device) candidate user information sets 15. In addition to or alternatively, example embodiments may configure user devices 10 to send/transmit/exchange candidate user information sets 15 to or with other user devices 10 (and/or location-based information sets 15′ from devices installed at particular locations 11′) via Bluetooth (or WiFi or any other form of wireless communications) so as to generate candidate contact instances 16 (either generate the candidate contact instances 16 by the other user device 10 or have the other user device send information to the processor 200 for the processor 200 to generate the candidate contact instances 16).

The Physical Exposure Processor (e.g., Element 230).

As illustrated in FIG. 2, an example embodiment of the processor 200 includes one or more physical exposure processors (e.g., physical exposure processors 230). The physical exposure processor 230 is configurable or configured to receive candidate contact instances 16 generated by the candidate contact instance generator 224 (and/or generated by user devices 10 and/or other devices, such as designated devices at particular locations 11, 11′, as described above and in the present disclosure). For each received candidate contact instance 16 (e.g., which, as described in the present disclosure, includes a candidate first user information set 15 for a first user 10 and a candidate second user information set 15 for a second user 10; and may also include one or more candidate other user information sets 15 for one or more other users 10, as described above and in the present disclosure), the physical exposure processor 230 is configurable or configured to perform an assessment of a likelihood that one or more of the users 10 (e.g., the second user 10) was physically exposed to a particular user 10 (e.g., the first user 10) during the candidate contact instance 16. It is to be understood in the present disclosure that references to an action or event (e.g., a physical exposure) occurring “during the candidate contact instance”, or the like, include references to the action or event (e.g., the physical exposure) occurring at the geolocation 11 and time 12 indicated in the candidate contact instance 16. It is also to be understood that the physical exposure processor 230 is configurable or configured to perform an assessment of a likelihood that one or more of the users 10 (e.g., the second user 10) was not physically exposed to a particular user 10 without departing from the teachings of the present disclosure.

In performing an analysis or assessment of a likelihood that a user 10 (e.g., the second user 10) was physically exposed to a particular user 10 (e.g., the first user 10) during a candidate contact instance 16, the physical exposure processor 230 is configurable or configured to derive an inaccuracy score for the candidate contact instance 16 and/or a non-contact score for the candidate contact instance 16.

In an example embodiment, the inaccuracy score for a candidate contact instance 16 represents a likelihood that the candidate contact instance 16 is inaccurate or incorrect (e.g., a likelihood that the distance between the geolocation 11 in the candidate first user information set 15 (for the first user 10) and the geolocation 11 in the candidate second user information set 15 (for the second user 10) is not less than the threshold distance value; a likelihood that the time between the timestamp 12 in the candidate first user information set 15 (for the first user 10) and the timestamp 12 in the candidate second user information set 15 (for the second user 10) is not less than the threshold time value).

In an example embodiment, the non-contact score for a candidate contact instance 16 represents a likelihood that the second user 10 was not physically exposed to the first user 10 during the candidate contact instance 16. The non-contact score may be derived based on one or more analyses. For example, the non-contact score may be derived based on a layout analysis (e.g., an analysis, based on one or more floor layouts, or the like, of a likelihood of a presence of a physical barrier, or the like, between the first user 10 and the second user 10 during the candidate contact instance 15). As used in the present disclosure, a “physical barrier” may be any part of any building, object, fixture, plant, or the like, that, if positioned between two users 10, may reduce a likelihood of a physical exposure (e.g., a physical exposure of viruses, bacteria, germs, airborne particles, liquid particles, radiation, contaminants, infectants, and/or the like) between the two users 10, and may include, but is not limited to, a wall, window, door, partition, screen, bookshelf, column/pillar, tree or plant, aquarium/water tank, curtain, vehicle, floor, ceiling, etc. Regarding the layout analysis, example embodiments include establishing, setting up, and/or configuring one or more floor layout databases and/or platforms 30, or the like, having floor layouts for buildings (e.g., office buildings, shopping malls, department stores, retail shops, restaurants, supermarkets, markets, sports complexes, sporting facilities, entertainment facilities, movie theatres, museums, residential homes, apartments, condominiums, places of worship, dormitories, etc.), transportation areas and/or hubs (e.g., train stations, bus stations/stops, taxi queues, airports, boat ports, streets, etc.), strip malls, neighborhoods, blocks, regions, parks, sporting facilities, playgrounds, other public places (e.g., squares, government centres, piazzas, etc.), public transportation vehicles (e.g., trains, street cars, buses, shuttles, airplanes, boats, etc.), etc. Such floor layout databases and/or platforms 30 may be similar to and/or be included/incorporated in map databases and/or platforms 30 (e.g., Google Maps, Apple Maps, etc.) so as to allow one or more elements of the processor 200 to perform searches for a floor layout (or several floor layouts) based on geolocation of users 10. Example embodiments of the layout analysis processor 242 (and/or one or more other elements of the processor 200, including processors 244-247) are then configurable or configured to perform an analysis (e.g., layout analysis, etc.) by first searching in the layout database 30 for one or more floor layouts having geolocations of the first and second users 10 (pursuant to the candidate contact instance 15), followed by deriving a non-contact score (e.g., based on a first physical barrier likelihood, etc.). Alternatively or in addition, the physical exposure processor 230 is configurable or configured to perform a map analysis (e.g., analysis, based on one or more maps, of a likelihood of a presence of a physical barrier (e.g., a wall, window, door, screen, floor, etc.), or the like, between the first user 10 and the second user 10 during the candidate contact instance 16). Alternatively or in addition, the physical exposure processor 230 is configurable or configured to perform a first transportation-based separation analysis (e.g., analysis, based on one or more maps, of a likelihood that the first user 10 was in a vehicle and/or the second user 10 was in a vehicle during the candidate contact instance 16). Alternatively or in addition, the physical exposure processor 230 is configurable or configured to perform a second transportation-based separation analysis (e.g., analysis, based on travel speeds of the first user 10 and the second user 10, of a likelihood that the first user 10 and the second user 10 were not traveling using the same transport mode during the candidate contact instance 16). Alternatively or in addition, the physical exposure processor 230 is configurable or configured to perform a third transportation-based separation analysis (e.g., analysis, based on travel directions of the first user 10 and second user 10, of a likelihood that the first user 10 was in a vehicle and/or the second user 10 was in a vehicle during the candidate contact instance 16). The physical exposure processor 230 is then configurable or configured to select one or more contact instances 16 from among the candidate contact instance 16 based on the derived non-contact score and/or derived inaccuracy score.

To perform these processes and/or methods, example embodiments of the physical exposure processor 230 include one or more elements. For example, as illustrated in FIG. 5, the physical exposure processor 230 includes one or more assessment interfaces 232. The physical exposure processor 230 also includes one or more inaccuracy processors 234. The physical exposure processor 230 also includes one or more non-contact processors 240. The physical exposure processor 230 also includes one or more contact instance selectors 236. Although the figures may illustrate one assessment interface 232, one inaccuracy processor 234, one non-contact processor 240, and one contact instance selector 236, it is to be understood in the present disclosure that the physical exposure processor 230 may include more or less than one assessment interface 232, more or less than one inaccuracy processor 234, more or less than one non-contact processor 240, and more or less than one contact instance selector 236 without departing from the teachings of the present disclosure. It is also to be understood in the present disclosure that, although the functions and/or processes performed by the physical exposure processor 230 are described in the present disclosure as being performed by particular elements of the physical exposure processor 230, the functions and/or processes performed by a particular element of the physical exposure processor 230 may also be performed by one or more other elements and/or cooperatively performed by more than one element of the processor 200 without departing from the teachings of the present disclosure. It is also to be understood in the present disclosure that, although the functions and/or processes performed by the physical exposure processor 230 are described in the present disclosure as being performed by particular elements of the physical exposure processor 230, the functions and/or processes performed by two or more particular elements of the physical exposure processor 230 may be combined and performed by one element of the processor 200 without departing from the teachings of the present disclosure.

These elements of the physical exposure processor 230 will now be further described with reference to the accompanying figures.

The Assessment Interface (e.g., Element 232).

As illustrated in FIG. 5, an example embodiment of the physical exposure processor 230 includes one or more assessment interfaces (e.g., assessment interface 232). The assessment interface 232 is configurable or configured to act as a communication interface for the physical exposure assessment processor 230 and obtain, request, and/or receive information from elements of the processor 200 and system 100. For example, the assessment interface 232 is configurable or configured to receive candidate contact instances 16 from the candidate contact instance generator 224. The assessment interface 232 is then configurable or configured to provide the candidate contact instances 16 to the inaccuracy processor 234, which performs an analysis of a likelihood that the candidate contact instance 16 is inaccurate or incorrect (e.g., a likelihood that the distance between the geolocation 11 in the candidate first user information set 15 (for the first user 10) and the geolocation 11 in the candidate second user information set 15 (for the second user 10) is not less than the threshold distance value; a likelihood that the time between the timestamp 12 in the candidate first user information set 15 (for the first user 10) and the timestamp 12 in the candidate second user information set 15 (for the second user 10) is not less than the threshold time value). To perform such an inaccuracy analysis, the assessment interface 232 is configurable or configured to obtain, request, and/or receive one or more threshold values for the inaccuracy processor 234 (e.g., threshold distance values from the threshold distance generator 203, threshold time values from the threshold time generator 204, and threshold inaccuracy values from the threshold inaccuracy generator 205).

The assessment interface 232 is also configurable or configured to provide the candidate contact instances 16 to the non-contact processor 240, which performs an analysis of a likelihood that a user 10 (e.g., the second user 10) was not physically exposed to one or more other users 10 (e.g., the first user 10) during the candidate contact instance 16. To perform such a non-contact analysis, the assessment interface 232 is configurable or configured to obtain, request, and/or receive one or more threshold values for the non-contact generator 240 (e.g., threshold physical barrier values from the threshold physical barrier generator 206a, threshold vehicular separation values from the threshold vehicular separation generator 206b, threshold transport mode values from the threshold transport mode generator 206c, threshold different level values from the threshold different level generator 206d, and threshold non-contact values from the threshold non-contact generator 206), one or more other user information sets 15, one or more other particular location information sets 15′, one or more other candidate contact instances 16, information from databases 30, and/or information from other elements of the processor 200 and/or other processors 200.

The assessment interface 232 is also configurable or configured to provide the candidate contact instances 16 to the databases 30, obtain, request, and/or receive information from the databases 30 and/or networks 20, and/or obtain, request, and/or receive instructions and/or information from administrators 40 and/or trusted authorities 40. The assessment interface 232 is also configurable or configured to obtain, request, and/or receive one or more threshold values for the contact instance selector 236 (e.g., threshold physical exposure values from the threshold physical exposure generator 207).

The Inaccuracy Processor (e.g., Element 234).

As illustrated in FIG. 5, an example embodiment of the physical exposure processor 230 includes one or more inaccuracy processors (e.g., inaccuracy processor 234). The inaccuracy processor 234 is configurable or configured to receive candidate contact instances 16 from the assessment interface 232, as well as other information from one or more elements of the processor 200 and/or system 100 including, but not limited to, information sets 15 from the candidate contact processor 220 (e.g., information sets 15 before and/or after the geolocations 11 and/or timestamps 12 in the candidate contact instances 16), information from one or more other elements of the processor 200 (e.g., threshold values from the generator 202), and/or other information from databases 30, networks 20, administrators 40, trusted authorities 40.

For each candidate contact instance 16 (including candidate contact instances 16 generated by the candidate contact processor 220 and/or user devices 10), the inaccuracy processor 234 is configurable or configured to derive/generate an inaccuracy score for each candidate contact instance 16 based on such received information. The inaccuracy score for each candidate contact instance represents a likelihood that the candidate contact instance 16 is inaccurate or incorrect.

In deriving an inaccuracy score for each candidate contact instance 16, the inaccuracy processor 234 is configurable or configured to derive a likelihood that the distance between the geolocation 11 in the candidate first user information set 15 (for the first user 10) and the geolocation 11 in the candidate second user information set 15 (for the second user 10), as derived by the candidate contact processor 220, is inaccurate and/or incorrect. For example, if the geolocation 11 for one or more of the users 10 in the candidate contact instance 16 was generated by the user device 10 based on a weaker signal and/or had a certain tolerance or error, then such uncertainty, tolerance, and/or error of the geolocation 11 for the one or more users 10 may increase the inaccuracy score for the candidate contact instance 16 (and/or result in the distance between the geolocations 11 for the users 10 to be a range of distance values instead of a single distance value). As another example, if a first geolocation 11 for a user 10 in the candidate contact instance 16 (which includes the first geolocation 11 for the user 10 and a first timestamp 12 for the user 10) deviates from and/or does not follow an expected/predicted path/pattern of other geolocations 11 for the user 10 (e.g., geolocations 11 for the user 10 before and/or after the candidate contact instance 16, that is before and/or after the first timestamp 12), then such deviated and/or unexpected/unpredicted first geolocation 11 for the user 10 may increase the inaccuracy score for the candidate contact instance 16 (and/or result in the distance between the geolocations 11 for the users 10 to be a range of distance values instead of a single distance value).

In addition or alternatively, the inaccuracy processor 234 is configurable or configured to derive a likelihood that the distance between the geolocation 11 in the candidate first user information set 15 (for the first user 10) and geolocation 11 in the candidate second user information set 15 (for the second user 10) is not less than a threshold distance value. The threshold distance value used to determine such likelihood may be dynamically generated by the threshold distance generator 203 for each candidate contact instance 16. Alternatively, the threshold distance generator 203 may provide a default or pre-selected threshold distance value to determine such likelihood.

In addition to or alternatively, the inaccuracy processor 234 is configurable or configured to derive a likelihood that the time between the timestamp 12 in the candidate first user information set 15 (for the first user 10) and the timestamp 12 in the candidate second user information set 15 (for the second user 10), as derived by the candidate contact processor 220, is inaccurate and/or incorrect. For example, if the timestamp 12 for one or more of the users 10 in the candidate contact instance 16 was generated by the user device 10 and such timestamp 12 deviates from a receipt time (e.g., time when the main interface 201 and/or one or more other elements of the processor 200 received the information set for the user 10), then such uncertainty, deviation, and/or error of the timestamp 12 for the one or more users 10 may increase the inaccuracy score for the candidate contact instance 16 (and/or result in the time between the timestamps 12 for the users 10 to be a range of time values instead of a single time value). As another example, if a first timestamp 12 for a user 10 in the candidate contact instance 16 (which includes a first geolocation 11 for the user 10 and the first timestamp 12 for the user 10) deviates from and/or does not follow an expected/predicted path/pattern of other timestamps 12 for the user 10 (e.g., timestamps 12 for the user 10 before and/or after the candidate contact instance 16, that is before and/or after the first timestamp 12), then such deviated and/or unexpected/unpredicted first timestamp 12 for the user 10 may increase the inaccuracy score for the candidate contact instance 16 (and/or result in the time between timestamps 12 for the users 10 to be a range of distance time instead of a single time value).

In addition to or alternatively, the inaccuracy processor 234 is configurable or configured to derive a likelihood that the time between the timestamp 12 in the candidate first user information set 15 (first user 10) and timestamp 12 in the candidate second user information set 15 (second user 10) is not less than a threshold time value. The threshold time value used to determine such likelihood may be dynamically generated by the threshold time generator 204 for each candidate contact instance 16. Alternatively, the threshold time generator 204 may provide a default or pre-selected threshold time value to determine such likelihood.

The inaccuracy score for each candidate contact instance 16 is then generated by the inaccuracy processor 234 based on one or more of the above likelihoods, including: the likelihood that the distance between the geolocation 11 in the candidate first user information set 15 (for the first user 10) and the geolocation 11 in the candidate second user information set 15 (for the second user 10), as derived by the candidate contact processor 220, is inaccurate and/or incorrect; the likelihood that the distance between the geolocation 11 in the candidate first user information set 15 (for the first user 10) and the geolocation 11 in the candidate second user information set 15 (for the second user 10), as derived by the candidate contact processor 220, is not less than the threshold distance value; the likelihood that the time between the timestamp 12 in the candidate first user information set 15 (for the first user 10) and the timestamp 12 in the candidate second user information set 15 (for the second user 10), as derived by the candidate contact processor 220, is inaccurate and/or incorrect; and/or the likelihood that the time between the timestamp 12 in the candidate first user information set 15 (for the first user 10) and the timestamp 12 in the candidate second user information set 15 (for the second user 10), as derived by the candidate contact processor 220, is not less than the threshold time value.

Once the inaccuracy score for each candidate contact instance 16 is derived/generated, the inaccuracy score is then provided to the contact instance selector 236 for use, in addition to or in replacement of the non-contact score of the candidate contact instance 16 generated by the non-contact processor 240 (as described below and in the present disclosure), to determine whether or not the candidate contact instance 16 should be selected for further processing by the contact instance notification processor 250.

The Non-Contact Processor (e.g., Element 240).

As illustrated in FIG. 5, an example embodiment of the physical exposure processor 230 includes one or more non-contact processors (e.g., non-contact processor 240). The non-contact processor 240 is configurable or configured to receive candidate contact instances 16 from the assessment interface 232, as well as other information from one or more elements of the processor 200 and/or system 100 including, but not limited to, information sets 15 from the candidate contact processor 220 (e.g., information sets 15 before and/or after geolocations 11 and/or timestamps 12 in the candidate contact instances 16), information from one or more other elements of the processor 200 (e.g., threshold values from generator 202), and/or other information from databases 30, networks 20, administrators/trusted authorities 40.

For each candidate contact instance 16 (including candidate contact instances 16 generated by the candidate contact processor 220 and/or user devices 10), the non-contact processor 240 is configurable or configured to derive/generate a non-contact score for the candidate contact instance 16. The non-contact score for a candidate contact instance 16 represents a likelihood that a user 10 (e.g., the second user 10) was not physically exposed to another user 10 (e.g., the first user 10) during the candidate contact instance 16. Alternatively or in addition, the non-contact processor 240 may also derive/generate a contact score for each candidate contact instance 16, which represents a likelihood that the second user was physically exposed to the first user during the candidate contact instance 16. In such example embodiments, if the non-contact score is in the form of a percentage probability of not being physically exposed (e.g., 25%), then the contact score will be a percentage probability of being physically exposed (e.g., 100%−25%, or 75%).

In an example embodiment, the likelihood that a user 10 (e.g., the second user 10) was physically exposed to another user 10 (e.g., the first user 10) during the candidate contact instance 16 is based on the non-contact score (or the contact score; and may also include a consideration of the inaccuracy score), and the non-contact score is derived based on at least one or more of the following: a layout analysis (e.g., an analysis, based on one or more floor layouts, of a likelihood of a presence of a physical barrier (e.g., a wall, window, door, screen, floor, etc.), or the like, between the first user 10 and the second user 10 during the candidate contact instance 16); and/or a map analysis (e.g., an analysis, based on one or more maps, of a likelihood of a presence of a physical barrier (e.g., a wall, window, door, screen, floor, etc.), or the like, between the first user 10 and the second user 10 during the candidate contact instance 16); and/or a first transportation-based separation analysis (e.g., an analysis, based on one or more maps, of a likelihood that the first user 10 was in a vehicle and/or the second user 10 was in a vehicle during the candidate contact instance 16); and/or a second transportation-based separation analysis (e.g., an analysis, based on travel speeds of the first user 10 and the second user 10, of a likelihood that the first user 10 and the second user 10 were not traveling using the same transport mode during the candidate contact instance 16); and/or a third transportation-based separation analysis (e.g., an analysis, based on travel directions of the first user 10 and the second user 10, of a likelihood that the first user 10 was in a vehicle and/or the second user 10 was in a vehicle during the candidate contact instance 16); and/or a different level analysis (e.g., an analysis, based on one or more floor layouts, a likelihood that the first user 10 and the second user 10 were on different levels/floors (e.g., different levels/floors of a building) during the candidate contact instance 16).

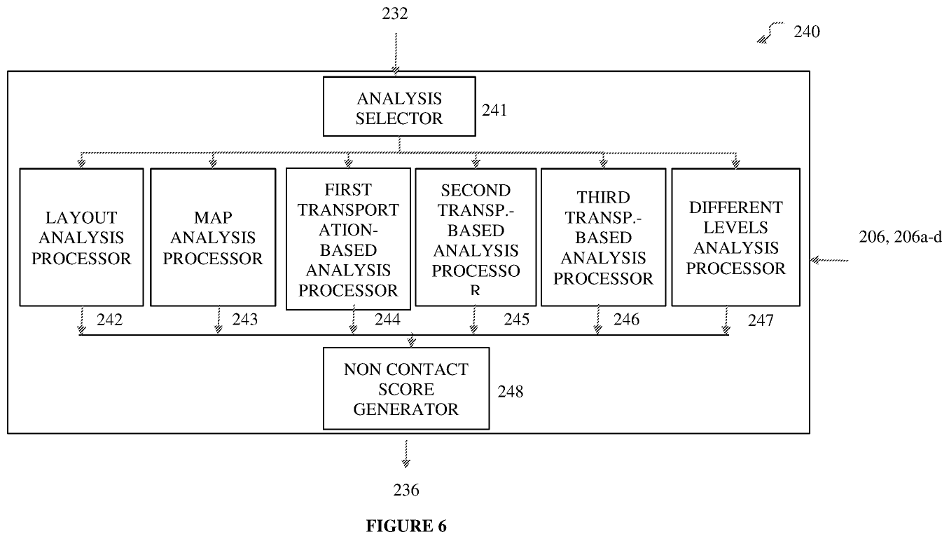

To perform these processes and/or methods, example embodiments of the non-contact processor 240 include one or more elements. For example, as illustrated in FIG. 6, the non-contact processor 240 includes one or more analysis selectors 241. The non-contact processor 240 also includes one or more layout analysis processors 242. The non-contact processor 240 also includes one or more map analysis processors 243. The non-contact processor 240 also includes one or more first transportation-based analysis processors 244. The non-contact processor 240 also includes one or more second transportation-based analysis processors 245. The non-contact processor 240 also includes one or more third transportation-based analysis processors 246. The non-contact processor 240 also includes one or more different levels analysis processors 247. The non-contact processor 240 also includes one or more non-contact score generators 248. Although the figures may illustrate one analysis selector 241, one layout analysis processor 242, one map analysis processor 243, one first transportation-based analysis processor 244, one second transportation-based analysis processor 245, one third transportation-based analysis processor 246, one different levels analysis processor 247, and one non-contact score generator 248, it is to be understood in the present disclosure that the non-contact processor 240 may include more or less than one analysis selector 241, more or less than one layout analysis processor 242, more or less than one map analysis processor 243, more or less than one first transportation-based analysis processor 244, more or less than one second transportation-based analysis processor 245, more or less than one third transportation-based analysis processor 246, more or less than one different levels analysis processor 247, and more or less than one non-contact score generator 248 without departing from the teachings of the present disclosure. It is also to be understood in the present disclosure that, although the functions and/or processes performed by the non-contact processor 240 are described in the present disclosure as being performed by particular elements of the non-contact processor 240, the functions and/or processes performed by a particular element of the non-contact processor 240 may also be performed by one or more other elements and/or cooperatively performed by more than one element of the processor 200 without departing from the teachings of the present disclosure. It is also to be understood in the present disclosure that, although the functions and/or processes performed by the non-contact processor 240 are described in the present disclosure as being performed by particular elements of the non-contact processor 240, the functions and/or processes performed by two or more particular elements of the physical exposure processor 230 may be combined and performed by one element of the processor 200 without departing from the teachings of the present disclosure.

These elements of the non-contact processor 240 will now be further described with reference to the accompanying figures.

i. The Analysis Selector (e.g., Element 241).

As illustrated in FIG. 6, an example embodiment of the non-contact processor 240 includes one or more analysis selectors (e.g., analysis selector 241). The analysis selector 241 is configurable or configured to receive candidate contact instances 16 from the assessment interface 232, as well as other information from one or more elements of the processor 200 and/or system 100 including, but not limited to, information sets 15 from the candidate contact processor 220 (e.g., information sets 15 before and/or after the geolocations 11 and/or timestamps 12 in the candidate contact instances 16), information from one or more other elements of the processor 200 (e.g., threshold values from the generator 202), and/or other information from databases 30, networks 20, administrators 40, trusted authorities 40.

The analysis selector 241 is then configurable to select one or more forms of analysis to perform on each of the received candidate contact instances 16 including, but not limited to: (1) a layout analysis by the layout analysis processor 242 (e.g., an analysis, based on one or more floor layouts, of a likelihood of a presence of a physical barrier (e.g., a wall, window, door, screen, floor, etc.), or the like, between a user 10 (e.g., the first user 10) and another user 10 (e.g., the second user 10) during the candidate contact instance 16); and/or (2) a map analysis by the map analysis processor 243 (e.g., an analysis, based on one or more maps, of a likelihood of a presence of a physical barrier (e.g., a wall, window, door, screen, floor, etc.), or the like, between a user 10 (e.g., the first user 10) and another user 10 (e.g., the second user 10) during the candidate contact instance 16); and/or (3) a first transportation-based separation analysis by the first transportation-based separation analysis processor 244 (e.g., an analysis, based on one or more maps, of a likelihood that a user 10 (e.g., the first user 10) was in a transportation-related vehicle and/or another user 10 (e.g., the second user 10) was in a transportation-related vehicle (and/or a different vehicle than the first user 10) during the candidate contact instance 16); and/or (4) a second transportation-based separation analysis by the second transportation-based separation analysis processor 245 (e.g., an analysis, based on travel speeds of a user 10 (e.g., the first user 10) and another user 10 (e.g., the second user 10), of a likelihood that the first user 10 and the second user 10 were not traveling using the same transport mode during the candidate contact instance 16 and/or a likelihood that the first user 10 was in a vehicle and/or the second user 10 was in a vehicle (and/or a different vehicle than the first user 10) during the candidate contact instance 16); and/or (5) a third transportation-based separation analysis by the third transportation-based separation analysis processor 246 (e.g., an analysis, based on travel directions of a user 10 (e.g., the first user 10) and another user 10 (e.g., the second user 10), of a likelihood that the first user 10 and the second user 10 were not traveling using the same transport mode during the candidate contact instance 16 and/or a likelihood that the first user 10 was in a vehicle and/or the second user 10 was in a vehicle (and/or different vehicle than the first user 10) during the candidate contact instance 16); and/or (6) a different level analysis by the different levels analysis processor 247 (e.g., an analysis, based on one or more floor layouts and/or maps, of a likelihood that a user 10 (e.g., the first user 10) and another user 10 (e.g., the second user 10) were on different levels/floors (e.g., different levels or floors of a building, such as a first floor vs a second floor of a shopping ball or office building) during the candidate contact instance 16).

For the one or more forms of analysis selected by the analysis selector 241 to perform on each of the received candidate contact instances 16, the analysis selector 241 (and/or the assessment interface 232 and/or other elements of the processor 200) is/are configurable or configured to provide the candidate contact instances 16 and other information to the processors 242, 243, 244, 245, 246, and/or 247 responsible for such selected analysis.

ii. The Layout Analysis Processor (e.g., Element 242).

As illustrated in FIG. 6, an example embodiment of the non-contact processor 240 includes one or more layout analysis processors (e.g., layout analysis processor 242). The layout analysis processor 242 is configurable or configured to receive candidate contact instances 16 and other information from the analysis selector 241 (and/or the assessment interface 232 and/or other elements of the processor 200). Each candidate contact instance 16 received by the layout analysis processor 242 includes a candidate information set 15 (for a user 10, which includes a geolocation 11 and timestamp 12 for the geolocation 11) and another candidate information set 15 (for another user 10, which includes a geolocation 11 and timestamp 12 for the geolocation 11). For example, the layout analysis processor 242 may receive a first candidate contact instance 16 having a candidate first user information set 15 (for a first user 10, which includes a first geolocation 11 for the first user 10 and first timestamp 12 for the first geolocation 11) and a candidate second user information set 15 (for a second user 10, which includes a second geolocation 11 for the second user 10 and second timestamp 12 for the second geolocation 11).

For each first candidate contact instance 16, the layout analysis processor 242 is configurable or configured to derive/generate a likelihood of a presence of a physical barrier between a first geolocation 11 (i.e., geolocation for a first user 10) and a second geolocation 11 (i.e., geolocation for a second user 10) during the first candidate contact instance 16 (e.g., at a time of a first timestamp 12 (i.e., timestamp for the first geolocation 11), at a time of a second timestamp 12 (i.e., timestamp for the second geolocation 11), and/or at one or more times before, between, and/or after the first timestamp 12 and/or the second timestamp 12) (referred to herein as the “first physical barrier likelihood”). As described above and in the present disclosure, the physical barrier may be and/or include any part of a building, object, fixture, living being or thing, naturally occurring thing, man-made thing, non-man-made thing, etc.; and such a physical barrier, if present during the first candidate contact instance 16 at a location (referred to herein as a “physical barrier location”, or the like) that corresponds to any location between the first user 10 (first geolocation 11) and second user 10 (second geolocation 11), would result in a reduced, lowered, and/or diminished likelihood of a physical exposure (e.g., a physical exposure of viruses, bacteria, germs, airborne particles, liquid particles, radiation, contaminants, infectants, contagions, etc.) between the first user 10 and second user 10. In an example embodiment, such a reduced likelihood refers to a reduced, lowered, or diminished likelihood when compared to a likelihood of physical exposure between the first user 10 and second user 10 in an alternative situation in which there was no physical barrier (or different physical barrier) present between the first user 10 (first geolocation 11) and second user 10 (second geolocation 11) during the first candidate contact instance 16. Examples of a physical barrier include, but are not limited to, a wall, window, door, elevator, elevator door, escalator, partition, screen, statue, bookshelf, column/pillar, tree, plant, rock formations, aquarium/water tank, curtain, vehicle, vehicle part (e.g., car door), animal (e.g., horse, etc.), one or more other users 10 (e.g., a third user 10 is standing between the first user 10 and the second user 10), floor, ceiling, etc. For example, a likelihood of physical exposure between the first user 10 (at the first geolocation 11) and the second user 10 (at the second geolocation 11) when a wall, window, and/or other physical barrier is located between the first user 10 (at the first geolocation 11) and the second user 10 (at the second geolocation 11) would be less than a likelihood of physical exposure between the first user 10 (at the first geolocation 11) and the second user 10 (at the second geolocation 11) if no wall, window, or any other physical barrier is located between the first user 10 (at the first geolocation 11) and the second user 10 (at the second geolocation 11).

The layout analysis processor 242 is configurable or configured to derive the first physical barrier likelihood by performing, among other things, an example embodiment of a layout analysis.

In an example embodiment, the layout analysis performed by the layout analysis processor 242 includes searching, in one or more databases 30 (e.g., a floor layout database 30, one or more sources on the internet (e.g., an internet-accessible repository of floor layouts), other information sources, etc.), for one or more floor layouts/floor plans (and/or maps and/or other information, including those described below and in the present disclosure), or the like, that cover the first geolocation 11 (for the first user 10) and the second geolocation 11 (for the second user 10) (or put differently, cover a geographic area or region that includes the first geolocation 11 and the second geolocation 11). As used in the present disclosure, a floor layout includes, but is not limited to, a drawing illustrating a layout view (or aerial view or view from the top) of rooms, sections, regions/areas, hallways/walkways, physical barriers (e.g., walls, windows, doors, columns, stairs, elevators, escalators, etc.), occupiable or walkable spaces, non-occupiable or non-walkable spaces, fixtures, furniture, and/or other physical features and/or objects; and such drawings are normally to scale. A floor layout may be in any form, format, file type, etc. including, but not limited to, the following file types: DWG, DXF, WMF, EMF, PNG, SVG, BMP, JPG, PDF, etc. For example, one or more parts of each floor layout may include geolocation information, labels, symbols, shapes, references, or the like, that enable the layout analysis processor 242 to determine whether or not the geographic area/region covered by the floor layout includes the first geolocation 11 and/or the second geolocation 11 (e.g., specific points on the floor layout may include geolocations (e.g., coordinates, or the like) and serve as reference points, or the like). Alternatively or in addition, the layout analysis processor 242 may be configurable or configured to: search, in one or more map databases 30, for one or more maps that include or cover the first geolocation 11 and second geolocation 11 (and/or a geographic area or region that includes the first geolocation 11 and second geolocation 11); search, in one or more floor layout databases 30, for one or more corresponding floor layouts that cover one or more geographic areas or regions in the one or more maps; and overlay, compare, and/or correlate the one or more floor layout(s) over or against (as applicable) the one or more map(s) (or vice versa) to determine and/or confirm the locations of the first geolocation 11 and second geolocation 11 on or within the floor layout(s). Alternatively or in addition, the layout analysis processor 242 may be configurable or configured to: search, in one or more floor layout databases 30, for one or more floor layouts; search, in one or more map databases 30, for one or more corresponding maps that include or cover the first geolocation 11 and second geolocation (and/or a geographic area or region that includes the first geolocation 11 and the second geolocation 11); and overlay, compare, and/or correlate the one or more floor layout(s) over or against (as applicable) the one or more map(s) (or vice versa) to determine and/or confirm the locations of the first geolocation 11 and second geolocation 11 on or within the floor layout(s). In example embodiments when more than one version, amendment, copy, etc. of a floor layout is found in the searches (e.g., changes to a floor layout), the search may include a consideration of date/time stamps, validity dates, revision/modification/update dates, etc. of the floor layouts in view of the first and second timestamps 12 to ensure appropriate floor layouts are selected.

It is to be understood in the present disclosure that the searches performed by the layout analysis processor 242 (and other elements of the non-contact processor 240) may also include searching for other information or using other ways (in addition to or alternatively to searches for floor layouts and/or maps) to determine whether or not a physical barrier is present at the physical barrier location. For example, example embodiments of the layout analysis processor 242 are also configurable or configured to search for image-related information (including video-related information) for one or more geographic areas or regions covering the first geolocation 11 (for the first user 10) and/or the second geolocation 11 (for the second user 10) including, but not limited to, CCTV videos of some or all parts of such geographic areas or regions, photos/images of such geographic areas or regions (e.g., photos/images searchable on the internet, photos/images tagged or linked to one or more geolocations in such geographic area (e.g., photos/images available in map applications, such as Google Maps), photos/images stored on or by a device of the first user 10 and/or second user 10 (e.g., provided the first user 10 and/or second user 10 provide authorization to search and/or access such photos/images), etc.), satellite images, etc. As used in the present disclosure, a floor layout may include, but is not limited to, conventional floor layouts and floor plans, floor layouts developed for the layout analysis processor 242 to more easily search and/or analyze, and/or other information (e.g., image-related information, etc. as described above and in the present disclosure).

After obtaining the one or more floor layouts (e.g., a first floor layout), the layout analysis performed by the layout analysis processor 242 further includes deriving, based on the one or more first floor layouts, a first physical barrier likelihood for the first candidate contact instance 16 (i.e., a likelihood that a physical barrier is present at a physical barrier location). The first physical barrier likelihood for the first candidate contact instance 16 is derived by first identifying a first location on the first floor layout. The first location on the first floor layout represent the first geolocation 11 of the first user 10. The layout analysis processor 242 then identifies a second location on the first floor layout. The second location on the first floor layout represent the second geolocation 11 of the second user 10. The layout analysis processor 242 then derives, based on the first and second locations on the first floor layout, a likelihood that a physical barrier is present between the first and second locations on the first floor layout. In this regard, the layout analysis processor 242 is configurable or configured to perform one or more of a plurality of analysis including, but not limited to, image recognition to identify lines, shapes, etc. that may correspond to physical barriers on the first floor layout (e.g., a wall, window, door, elevator, stairs, etc.), character recognition (e.g., OCR) to identify textual descriptions and/or labels for physical barriers on the first floor layout, etc. For example, the layout analysis processor 242 may include and/or cooperate with one or more forms of artificial intelligence (AI) (e.g., machine learning or deep learning via convolution neural networks for image recognition, etc.) to determine the likelihood that lines, shapes, figures, characters, symbols, etc. found in the first floor layout represent and/or refer to physical barriers. After performing such analysis to identify one or more physical barriers on the first floor layout, the layout analysis processor 242 then derives/generates the first physical barrier likelihood, which includes one or more of the following likelihood components: a likelihood that the physical barrier identified by such analysis is indeed a physical barrier; a likelihood that the physical barrier identified by such analysis is not located at the physical barrier location (i.e., not at a location between the first geolocation 11 and the second geolocation 11); a likelihood that the first floor layout is not an accurate or correct floor layout; a likelihood that the location of the physical barrier, the first geolocation 11 of the first user 10, the second geolocation 11 of the second user 10, and/or the physical barrier location is/are inaccurate and/or not correct. In an example embodiment, the likelihood components may have equal or different weighting or value.

FIG. 7A illustrates some examples of first locations (i.e., first geolocation 11 for the first user 10) and second locations (i.e., second geolocation 11 for the second user 10) on a first floor layout. In a first example, first location 10a (i.e., the first user) and second location 10b (i.e., the second user) are identified on the first floor layout without any physical barrier between the first location 10a and the second location 10b. In this first example, the first physical barrier likelihood will be very low (or zero), the non-contact score for the first contact instance 16 will be very low (or zero), and the risk or likelihood of the second user being physically exposed to the first user (or vice versa) will be very high. In a second example, first location 10c (i.e., the first user) and second location 10d (i.e., the second user) are identified on the first floor layout without any physical barrier between the first location 10c and the second location 10d. However, the first and second locations 10c and 10d are closer to a wall 50c as compared to the first example. In this second example, the first physical barrier likelihood will be low (but slightly higher than the first example because the locations 10c, 10d are closer to a physical barrier (wall) than the locations 10a, 10b), the non-contact score for the first contact instance 16 will be low (also slightly higher than the first example), and the risk or likelihood of the second user being physically exposed to the first user (or vice versa) will be high (but lower than the first example). In a third example, first location 10e (i.e., the first user) and second location 10f (i.e., the second user) are identified on the first floor layout with physical barrier 50b between the first location 10a and the second location 10b. In this third example, the first physical barrier likelihood will be very high, the non-contact score for the first contact instance 16 will be very high, and the risk or likelihood of the second user being physically exposed to the first user (or vice versa) will be very low. In a fourth example, first location 10g (i.e., the first user) and second location 10h (i.e., the second user) are identified on the first floor layout without any physical barrier between the first location 10g and the second location 10h. However, the first and second locations 10g and 10h are closer to a wall 50a as compared to the first and second examples. In this fourth example, the first physical barrier likelihood will be low (but slightly higher than the second example because a distance from the locations 10g, 10h to the physical barrier (wall) 50a is closer than a distance from the locations 10c, 10d to the physical barrier (wall) 50c), the non-contact score for the first contact instance 16 will be low (slightly higher than the second example), and the risk or likelihood of the second user being physically exposed to the first user (or vice versa) will be high (lower than the second example).

After the first physical barrier likelihood is derived/generated, the layout analysis processor 242 is configurable or configured to obtain a threshold physical barrier value from the threshold physical barrier generator 206a (e.g., via the main threshold processor 202′). The threshold physical barrier value may be dynamically generated by the threshold physical barrier generator 206a based on one or more considerations including, but not limited to, one or more of the likelihood components for the first physical barrier likelihood, one or more aspects of the first candidate contact instance 16, etc. Alternatively or in addition, the threshold physical barrier value may be a default or predetermined threshold value (or selected from one or more default and/or predetermined threshold values). The layout analysis processor 242 is then configurable or configured to compare the first physical barrier likelihood with the threshold physical barrier value.

In an example embodiment, the layout analysis processor 242 is then configurable or configured to provide the first physical barrier likelihood for the first candidate contact instance 16 to the non-contact score generator 248 for use in deriving the non-contact score for the first candidate contact instance 16 (e.g., along with one or more results from other elements of the non-contact processor 240, including the map analysis processor 243, the first transportation-based analysis processor 244, the second transportation-based analysis processor 245, the third transportation-based analysis processor 246, and/or the different levels analysis processor 247). In example embodiments, the layout analysis processor 242 provides the first physical barrier likelihood to the non-contact score generator 248 based on a comparison of the first physical barrier likelihood with the threshold physical barrier value. For example, the layout analysis processor 242 provides the first physical barrier likelihood to the non-contact score generator 248 when the first physical barrier likelihood is greater than or equal to the threshold physical barrier value.

iii. The Map Analysis Processor (e.g., Element 243).

As illustrated in FIG. 6, an example embodiment of the non-contact processor 240 includes one or more map analysis processors (e.g., map analysis processor 243). The map analysis processor 243 is configurable or configured to receive candidate contact instances 16 and other information from the analysis selector 241 (and/or the assessment interface 232 and/or other elements of the processor 200). Each candidate contact instance 16 received by the map analysis processor 243 includes a candidate information set 15 (for a user 10, which includes a geolocation 11 and a timestamp 12 for the geolocation 11) and another candidate information set 15 (for another user 10, which includes a geolocation 11 and a timestamp 12 for the geolocation 11). For example, the map analysis processor 243 may receive a first candidate contact instance 16 having a candidate first user information set 15 (for a first user 10, which includes a first geolocation 11 for the first user 10 and a first timestamp 12 for the first geolocation 11) and a candidate second user information set 15 (for a second user 10, which includes a second geolocation 11 for the second user 10 and a second timestamp 12 for the second geolocation 11).

For each first candidate contact instance 16, the map analysis processor 243 is configurable or configured to derive/generate a likelihood of a presence of a physical barrier between a first geolocation 11 (i.e., geolocation for a first user 10) and a second geolocation 11 (i.e., geolocation for a second user 10) during the first candidate contact instance 16 (e.g., at a time of a first timestamp 12 (i.e., timestamp for the first geolocation 11), at a time of a second timestamp 12 (i.e., timestamp for the second geolocation 11), and/or at one or more times before, between, and/or after the first timestamp 12 and/or second timestamp 12) (referred to herein as the “second physical barrier likelihood”). In example embodiments, the map analysis processor 243 may also derive the second physical barrier likelihood by searching and analyzing not just floor layouts, but also maps.

The map analysis processor 243 is configurable or configured to derive the second physical barrier likelihood by performing, among other things, an example embodiment of a map analysis.

In an example embodiment, the map analysis performed by the map analysis processor 243 includes searching, in one or more databases 30 (e.g., a map database 30, one or more sources on the internet (e.g., an internet-accessible repository of maps), other information sources, etc.), for one or more maps (and/or floor layouts and/or other information, including those described below and in the present disclosure; referred to herein as a “map”, or the like), or the like, that cover the first geolocation 11 (for the first user 10) and the second geolocation 11 (for the second user 10) (or put differently, cover a geographic area or region that includes the first geolocation 11 and the second geolocation 11). For example, one or more parts of each map may include geolocation information, labels, symbols, references, or the like, that enable the map analysis processor 243 to determine whether or not the geographic area/region covered by the map includes the first geolocation 11 and/or the second geolocation 11 (e.g., specific points on the map may include geolocations (e.g., coordinates, or the like) and serve as reference points, or the like). Alternatively or in addition, the map analysis processor 243 may be configurable or configured to: search, in one or more map databases 30, for one or more maps that include or cover the first geolocation 11 and the second geolocation (and/or a geographic area or region that includes the first geolocation 11 and the second geolocation 11); search, in one or more floor layout databases 30, for one or more corresponding floor layouts that cover one or more geographic areas or regions in the one or more maps; and overlay, compare, and/or correlate the one or more floor layout(s) over or against (as applicable) the one or more map(s) (or vice versa) to determine and/or confirm the locations of the first geolocation 11 and second geolocation 11 on or within the floor layout(s). Alternatively or in addition, the map analysis processor 243 may be configurable or configured to: search, in one or more floor layout databases 30, for one or more floor layouts; search, in one or more map databases 30, for one or more corresponding maps that include or cover the first geolocation 11 and the second geolocation (and/or a geographic area or region that includes the first geolocation 11 and the second geolocation 11); and overlay, compare, and/or correlate the one or more floor layout(s) over or against (as applicable) the one or more map(s) (or vice versa) to determine and/or confirm the locations of the first geolocation 11 and second geolocation 11 on or within the floor layout(s). In example embodiments when more than one version, amendment, copy, etc. of a map is found in the searches (e.g., changes to a map), the search may include a consideration of date/time stamps, validity dates, revision/modification/update dates, etc. of the map in view of the first and second timestamps 12 to ensure appropriate maps are selected.

It is to be understood in the present disclosure that the searches performed by the map analysis processor 243 (and other elements of the non-contact processor 240) may also include searching for other information or using other ways (in addition to or alternatively to searches for floor layouts and/or maps) to determine whether or not a physical barrier is present at the physical barrier location. For example, example embodiments of the map analysis processor 243 are also configurable or configured to search for image-related information (including video-related information) for one or more geographic areas or regions covering the first geolocation 11 (for the first user 10) and/or second geolocation 11 (for the second user 10), as described above for the layout analysis processor 242. As used in the present disclosure, a map may include conventional maps (e.g., Google Maps, Apple Maps, etc.), maps developed for the map analysis processor 243 to more easily search and/or analyze, satellite maps, simplified maps, and/or other information (e.g., image-related information, etc. as described above and in the present disclosure).

After obtaining the one or more maps (e.g., a first map), the map analysis performed by the map analysis processor 243 further includes deriving, based on the one or more first map layouts, the second physical barrier likelihood for the first candidate contact instance 16 (i.e., a likelihood that a physical barrier is present at the physical barrier location). The second physical barrier likelihood for the first candidate contact instance 16 is derived by first identifying a first location on the first map. The first location on the first map represent the first geolocation 11 of the first user 10. The map analysis processor 243 then identifies a second location on the first map. The second location on the first map represent the second geolocation 11 of the second user 10. The map analysis processor 243 then derives, based on the first and second locations on the first map, a likelihood that a physical barrier is present between the first and second locations on the first map. In this regard, the map analysis processor 243 is configurable or configured to perform one or more of a plurality of analysis including, but not limited to, image recognition to identify lines, shapes, etc. that may correspond to physical barriers on the first map (e.g., a wall, window, door, elevator, stairs, etc.), character recognition (e.g., OCR) to identify textual descriptions and/or labels for physical barriers on the first map, etc. For example, the map analysis processor 243 may include and/or cooperate with one or more forms of artificial intelligence (AI) (e.g., machine learning or deep learning via convolution neural networks for image recognition, etc.) to determine the likelihood that lines, shapes, figures, characters, symbols, etc. found in the first map represent and/or refer to physical barriers. After performing such analysis to identify one or more physical barriers on the first map, the map analysis processor 243 then derives/generates the second physical barrier likelihood, which includes one or more of the following likelihood components: a likelihood that the physical barrier identified by such analysis is indeed a physical barrier; a likelihood that the physical barrier identified by such analysis is not located at the physical barrier location (i.e., not at a location between the first geolocation 11 and the second geolocation 11); a likelihood that the first map is not an accurate or correct map; a likelihood that the location of the physical barrier, the first geolocation 11 of the first user 10, the second geolocation 11 of the second user 10, and/or the physical barrier location is/are inaccurate and/or not correct. In an example embodiment, the likelihood components may have equal or different weighting or value.

FIG. 7B illustrates some examples of first locations (i.e., first geolocation 11 for the first user 10) and second locations (i.e., second geolocation 11 for the second user 10) on a first map. In a first example, first location 10a (i.e., the first user) and second location 10b (i.e., the second user) are identified on the first map without any physical barrier between the first location 10a and the second location 10b. In this first example, the first physical barrier likelihood will be very low (or zero), the non-contact score for the first contact instance 16 will be very low (or zero), and the risk or likelihood of the second user being physically exposed to the first user (or vice versa) will be very high. In a second example, first location 10c (i.e., the first user) and second location 10d (i.e., the second user) are identified on the first floor layout without any physical barrier between the first location 10c and the second location 10d. However, the first and second locations 10c and 10d are closer to physical barrier (building wall) 50c as compared to the first example. In this second example, the first physical barrier likelihood will be low (but slightly higher than the first example because the locations 10c, 10d are closer to a physical barrier (building wall) than the locations 10a, 10b), the non-contact score for the first contact instance 16 will be low (also slightly higher than the first example), and the risk or likelihood of the second user being physically exposed to the first user (or vice versa) will be high (but lower than the first example). In a third example, first location 10e (i.e., the first user) and second location 10f (i.e., the second user) are identified on the first floor layout with physical barrier (building wall) 50c between the first location 10a and the second location 10b. In this third example, the first physical barrier likelihood will be very high, the non-contact score for the first contact instance 16 will be very high, and the risk or likelihood of the second user being physically exposed to the first user (or vice versa) will be very low. In a fourth example, first location 10g (i.e., the first user) and second location 10h (i.e., the second user) are identified on the first floor layout without any physical barrier between the first location 10g and the second location 10h. However, the first and second locations 10g and 10h are closer to a physical barrier (building wall) 50c as compared to the first example. In this fourth example, the first physical barrier likelihood will be low (but slightly higher than the first example because a distance from the locations 10g, 10h to the physical barrier (building wall) 50c is closer than a distance from the locations 10a, 10b to the physical barrier (building wall) 50c), the non-contact score for the first contact instance 16 will be low (also slightly higher than the first example), and the risk or likelihood of the second user being physically exposed to the first user (or vice versa) will be high (but lower than the first example).

After the second physical barrier likelihood is derived/generated, the map analysis processor 243 is configurable or configured to obtain a threshold physical barrier value from the threshold physical barrier generator 206a (e.g., via main threshold processor 202′), as described above for the layout analysis processor 242. The map analysis processor 243 is then configured to compare the second physical barrier likelihood with the threshold physical barrier value.

In an example embodiment, the map analysis processor 243 is then configurable or configured to provide the second physical barrier likelihood for the first candidate contact instance 16 to the non-contact score generator 248 for use in deriving the non-contact score for the first candidate contact instance 16 (e.g., along with one or more results from other elements of the non-contact processor 240, including the layout analysis processor 242, the first transportation-based analysis processor 244, the second transportation-based analysis processor 245, the third transportation-based analysis processor 246, and/or the different levels analysis processor 247). In example embodiments, the map analysis processor 243 provides the second physical barrier likelihood to the non-contact score generator 248 based on a comparison of the second physical barrier likelihood with the threshold physical barrier value. For example, the map analysis processor 243 provides the second physical barrier likelihood to the non-contact score generator 248 when the second physical barrier likelihood is greater than or equal to the threshold physical barrier value.

iv. The First Transportation-Based Analysis Processor (e.g., Element 244).

As illustrated in FIG. 6, an example embodiment of the non-contact processor 240 includes one or more first transportation-based analysis processors (e.g., first transportation-based analysis processor 244). The first transportation-based analysis processor 244 is configurable or configured to receive candidate contact instances 16 and other information from the analysis selector 241 (and/or the assessment interface 232 and/or other elements of the processor 200). Each candidate contact instance 16 received by the map analysis processor 243 includes a candidate information set 15 (for a user 10, which includes a geolocation 11 and a timestamp 12 for the geolocation 11) and another candidate information set 15 (for another user 10, which includes a geolocation 11 and a timestamp 12 for the geolocation 11). For example, the first transportation-based analysis processor 244 may receive a first candidate contact instance 16 having a candidate first user information set 15 (for a first user 10, which includes a first geolocation 11 for the first user 10 and a first timestamp 12 for the first geolocation 11) and a candidate second user information set 15 (for a second user 10, which includes a second geolocation 11 for the second user 10 and a second timestamp 12 for the second geolocation 11).

For each first candidate contact instance 16, the first transportation-based analysis processor 244 is configurable or configured to derive/generate a likelihood that: the first user 10 was in a vehicle (e.g., car, SUV, truck, RV, bus, airplane, train, boat, hyperloop, or other vehicles) at a time and/or location indicated in the candidate first user information set 15 (i.e., at a time corresponding to the first timestamp 12; and when the first geolocation 11 was recorded); and/or the second user 10 was in a vehicle (e.g., car, SUV, truck, RV, bus, airplane, train, boat, hyperloop, or other vehicles) at a time and/or location indicated in the candidate second user information set 15 (i.e., at a time corresponding to the second timestamp 12; and when the second geolocation 11 was recorded); and/or at one or more times and/or locations before and/or after the aforementioned first timestamp 12, second timestamp 12, first geolocation 11, and/or second geolocation 11 (referred to herein as the “vehicular separation likelihood”).

The first transportation-based analysis processor 244 is configured to derive the vehicular separation likelihood by performing, among other things, an example embodiment of a first transportation-based separation analysis (or first transportation-based analysis).

In an example embodiment, the first transportation-based separation analysis performed by the first transportation-based analysis processor 244 includes searching, in one or more databases 30 (e.g., a map database 30, one or more sources on the internet (e.g., an internet-accessible repository of maps), other information sources, etc.), for one or more maps (and/or city plans and/or other information, including those described below and in the present disclosure), or the like, that cover the first geolocation 11 (for the first user 10) and the second geolocation 11 (for the second user 10) (or put differently, cover a geographic area or region that includes the first geolocation 11 and the second geolocation 11). For example, one or more parts of each map may include geolocation information, labels, symbols, references, or the like, that enable the first transportation-based analysis processor 244 to determine whether or not the geographic area/region covered by the map includes the first geolocation 11 and/or the second geolocation 11 (e.g., specific points on the map may include geolocations (e.g., coordinates, or the like) and serve as reference points, or the like). Alternatively or in addition, the first transportation-based analysis processor 244 may be configurable or configured to: search, in one or more map databases 30, for one or more maps that include or cover the first geolocation 11 and the second geolocation (and/or a geographic area or region that includes the first geolocation 11 and the second geolocation 11); search, in one or more satellite image or vehicular traffic databases 30, for one or more corresponding satellite and/or vehicle traffic images (e.g., images indicating the amount of traffic on roads and other pathways intended for vehicular travel) that cover one or more geographic areas or regions in the one or more maps; and overlay, compare, and/or correlate the one or more satellite and/or vehicle traffic images over or against (as applicable) the one or more map(s) (or vice versa) to determine and/or confirm the locations of the first geolocation 11 and second geolocation 11 on or within the satellite and/or vehicle traffic images. Alternatively or in addition, the first transportation-based analysis processor 244 may be configurable or configured to: search, in one or more satellite and/or vehicle traffic databases 30, for one or more satellite and/or vehicle traffic images; search, in one or more map databases 30, for one or more corresponding maps that include or cover the first geolocation 11 and the second geolocation (and/or a geographic area or region that includes the first geolocation 11 and the second geolocation 11); and overlay, compare, and/or correlate the one or more satellite and/or vehicle traffic images over or against (as applicable) the one or more map(s) (or vice versa) to determine and/or confirm the locations of the first geolocation 11 and second geolocation 11 on or within the satellite and/or vehicle traffic images. In example embodiments when more than one version, amendment, copy, etc. of a map, satellite image, and/or vehicle traffic image is found in the searches (e.g., changes, updates, etc.), the search may include a consideration of date/time stamps, validity dates, revision/modification/update dates, etc. of the image in view of the first and second timestamps 12 to ensure appropriate images are selected.

It is to be understood in the present disclosure that the searches performed by the first transportation-based analysis processor 244 (and other elements of the non-contact processor 240) may also include searching for other information or using other ways (in addition to or alternatively to searches for maps, satellite images, and/or vehicle traffic images) to determine whether or not the first user 10 was in a vehicle and/or the second user 10 was in a transportation vehicle during the first contact instance 16. For example, example embodiments of the first transportation-based analysis processor 244 are also configurable or configured to search for image-related information (including video-related information) for one or more geographic areas or regions covering the first geolocation 11 (for first user 10) and/or the second geolocation 11 (for second user 10), as described above for the layout analysis processor 242.

After obtaining the one or more maps (e.g., a first map), the first transportation-based separation analysis performed by the first transportation-based analysis processor 244 further includes deriving, based on the one or more first map layouts, the vehicular separation likelihood for the first candidate contact instance 16 (i.e., a likelihood that the first user 10 was in a vehicle and/or the second user 10 was in a vehicle during the first contact instance 16). The vehicular separation likelihood for the first candidate contact instance 16 is derived by first identifying a first location on the first map. The first location on the first map represent the first geolocation 11 of the first user 10. The first transportation-based analysis processor 244 then identifies a second location on the first map. The second location on the first map represent the second geolocation 11 of the second user 10. The first transportation-based analysis processor 244 then derives, based on the first location on the first map, a likelihood that the first location is within a first transport area on the first map. The first transportation-based analysis processor 244 also derives, based on the second location on the first map, a likelihood that the second location is within a second transport area on the first map. As used in the present disclosure, a transport area on a map is an area on the map intended as a vehicle transportation area, and includes, but is not limited to, a road, street, avenue, boulevard, highway, freeway, tollway, parking lot, alley, drive-thru (e.g., for a restaurant, supermarket, pharmacy, ATM, disease or virus testing facility, or the like), car wash, etc. Accordingly, a “first transport area” and “second transport area” on the first map will refer to areas on the first map intended as a vehicle transportation area. When needed, the first transportation-based analysis processor 244 may perform one or more analyses including, but not limited to, image recognition to identify lines, shapes, patterns, etc. that may correspond to vehicle transportation areas on the first map, character recognition (e.g., OCR) to identify textual descriptions and/or labels for vehicle transportation areas on the first map, etc. As example, the first transportation-based analysis processor 244 may include and/or cooperate with one or more forms of artificial intelligence (AI) (e.g., machine learning or deep learning via convolution neural networks for image recognition, etc.), or the like, that are trained (via maps) and applied to identify regions in a map intended for transportation (e.g., roads, highways, railroad tracks, subway tracks, canals/rivers, etc.), or the like, from lines, shapes, symbols, markings, figures, characters, or the like, found in maps. After performing such analysis to identify one or more vehicle transportation areas on the first map, the first transportation-based analysis processor 244 then derives/generates the vehicular separation likelihood, which includes one or more of the following likelihood components: a likelihood that the first location is in or within a first transport area; a likelihood that the first user 10 was in a vehicle during the first contact instance 16; a likelihood that the second location is in or within a second transport area; a likelihood that the second user 10 was in a vehicle during the first contact instance 16; a likelihood that the first user 10 and second user 10 were in different vehicles during the first user contact instance 16; a likelihood that the first user 10 and the second user 10 were in the same vehicle during the first user contact instance 16; a likelihood that the first map is not an accurate or correct map; a likelihood that the location of the first transport area, the second transport area, the first geolocation 11 of the first user 10, and/or the second geolocation 11 of the second user 10 is/are inaccurate and/or not correct. In an example embodiment, the likelihood components may have equal or different weighting or value.

Referring back to FIG. 7B, which illustrates some examples of first locations (i.e., first geolocation 11 for the first user 10) and second locations (i.e., second geolocation 11 for the second user 10) on the first map. In a first example for the first transportation-based analysis processor 244, first location 10i (i.e., the first user) is identified on the first map as being within first transport area 51a and second location 10j (i.e., the second user) is identified on the first map as being within second transport area 51b. In this first example, the vehicular separation likelihood will be high (there is a very high likelihood that the first user is in a vehicle; there is a very high likelihood the second user is in a vehicle; there is a high likelihood that the first and second users are in different vehicles since they are on different lanes (but the lanes are in the same direction, which leaves open a possibility that the first and second users are in the same vehicle and the vehicle is changing lanes)), the non-contact score for the first contact instance 16 will be high, and the risk or likelihood of the second user being physically exposed to the first user (or vice versa) will be low. In a second example for the first transportation-based analysis processor 244, first location 10j (i.e., the first user) is identified on the first map as being within first transport area 51b and second location 10k (i.e., the second user) is identified on the first map as not being within any transport area (since the second user is identified as being on a sidewalk and not a vehicular transportation area; which may be determined by also identifying boundary 50a). In this second example, the vehicular separation likelihood will be very high (there is a very high likelihood that the first user is in a vehicle; there is a very low likelihood the second user is in a vehicle; and there is a very high likelihood that the first and second users are not in the same vehicle), the non-contact score for the first contact instance 16 will be very high, and the risk or likelihood of the second user being physically exposed to the first user (or vice versa) will be very low. In a third example for the first transportation-based analysis processor 244, first location 10l (i.e., the first user) is identified on the first map as being within third transport area 52a and second location 10m (i.e., the second user) is identified on the first map as also being within third transport area 52a. In this third example, the vehicular separation likelihood will be medium (there is a very high likelihood that the first user is in a vehicle; there is a very high likelihood the second user is in a vehicle; there is a medium likelihood that the first and second users are in separate vehicles, but also a medium likelihood that the first and second users are in a same vehicle (e.g., a van or bus)), the non-contact score for the first contact instance 16 will be medium, and the risk or likelihood of the second user being physically exposed to the first user (or vice versa) will be medium. In a fourth example for the first transportation-based analysis processor 244, first location 10n (i.e., the first user) is identified on the first map as being within fourth transport area 52b and second location 10p (i.e., the second user) is identified on the first map as being within fifth transport area 53a. In this fourth example, the vehicular separation likelihood will be very high (there is a very high likelihood that the first user is in a vehicle; there is a very high likelihood the second user is in a vehicle; there is a very high likelihood that the first and second users are in different vehicles since they are on different lanes (which are lanes in the opposite direction)), the non-contact score for the first contact instance 16 will be very high, and the risk or likelihood of the second user being physically exposed to the first user (or vice versa) will be very low. In a fifth example for the first transportation-based analysis processor 244, first location 10q (i.e., the first user) is identified on the first map as being within fifth transport area 53a and second location 10r (i.e., the second user) is identified on the first map as being within fifth transport area 53a. In this fifth example, the vehicular separation likelihood will be very low (there is a very high likelihood that the first user is in a vehicle; there is a very high likelihood the second user is in a vehicle; but there is a very low likelihood that the first and second users are in different vehicles since they both in the same lane), the non-contact score for the first contact instance 16 will be very low, and the risk or likelihood of the second user being physically exposed to the first user (or vice versa) will be very high.

After the vehicular separation likelihood is derived/generated, the first transportation-based analysis processor 244 is configurable or configured to obtain a threshold vehicular separation value from the threshold vehicular separation generator 206b (e.g., via main threshold processor 202′). The threshold vehicular separation value may be dynamically generated by the threshold vehicular separation generator 206b based on one or more considerations including, but not limited to, one or more of the likelihood components for the vehicular separation likelihood, one or more aspects of the first candidate contact instance 16, etc. Alternatively or in addition, the threshold vehicular separation value may be a default or predetermined threshold value (or selected from one or more default and/or predetermined threshold values). The first transportation-based analysis processor 244 is then configured to compare the vehicular separation likelihood with the threshold vehicular separation value.

In an example embodiment, the first transportation-based analysis processor 244 is then configurable or configured to provide the vehicular separation likelihood for the first candidate contact instance 16 to the non-contact score generator 248 for use in deriving the non-contact score for the first candidate contact instance 16 (e.g., along with one or more results from other elements of the non-contact processor 240, including the layout analysis processor 242, the map analysis processor 243, the second transportation-based analysis processor 245, the third transportation-based analysis processor 246, and/or the different levels analysis processor 247). In example embodiments, the first transportation-based analysis processor 244 provides the vehicular separation likelihood to the non-contact score generator 248 based on a comparison of the vehicular separation likelihood with the threshold vehicular separation value. For example, the first transportation-based analysis processor 244 provides the vehicular separation likelihood to the non-contact score generator 248 when the vehicular separation likelihood is greater than or equal to the threshold vehicular separation value.

v. The Second Transportation-Based Analysis Processor (e.g., Element 245).

As illustrated in FIG. 6, an example embodiment of the non-contact processor 240 includes one or more second transportation-based analysis processors (e.g., second transportation-based analysis processor 245). The second transportation-based analysis processor 245 is configurable or configured to receive candidate contact instances 16 and other information from the analysis selector 241 (and/or the assessment interface 232 and/or other elements of the processor 200). Each candidate contact instance 16 received by the second transportation-based analysis processor 245 includes a candidate information set 15 (for a user 10, which includes a geolocation 11 and a timestamp 12 for the geolocation 11) and another candidate information set 15 (for another user 10, which includes a geolocation 11 and a timestamp 12 for the geolocation 11). For example, the second transportation-based analysis processor 245 may receive a first candidate contact instance 16 having a candidate first user information set 15 (for a first user 10, which includes a first geolocation 11 for the first user 10 and a first timestamp 12 for the first geolocation 11) and a candidate second user information set 15 (for a second user 10, which includes a second geolocation 11 for the second user 10 and a second timestamp 12 for the second geolocation 11).

For each first candidate contact instance 16, the second transportation-based analysis processor 245 is configurable or configured to derive/generate a likelihood that a user 10 (e.g., the first user 10) and another user 10 (e.g., the second user 10) were not traveling using the same transport mode (e.g., not traveling using a common transport mode; traveling using different transport modes; one of the users 10 traveling using one transport mode and the other user 10 not traveling (stationary); etc.) (such likelihood referred to herein as the “first different transport mode likelihood”). As used in the present disclosure, a “transport mode”, or the like, for a user 10 may refer to any mode of transportation for the user 10 including, but not limited to, traveling by foot (e.g., walking, jogging, running, etc.) and traveling via some form of transportation vehicle or device (e.g., bicycle, scooter, car, taxi, bus, train, boat, airplane, etc.). In this regard, the first and second users 10 may be considered as traveling using the same transport mode when the first and second users 10 are both walking, both in the same vehicle, etc. Similarly, the first and second users 10 may be considered as not traveling using the same transport mode when the first user 10 is walking and the second user 10 is traveling by car; the first user 10 is walking and the second user 10 is jogging or cycling; the first user 10 is traveling in a first car and the second user is traveling in a second car different from the first car; the first user 10 is walking and the second user 10 is stationary (not moving); etc.

The second transportation-based analysis processor 245 is configured to derive the first different transport mode likelihood by performing, among other things, an example embodiment of a second transportation-based separation analysis (or second transportation-based analysis).

In an example embodiment, the second transportation-based separation analysis performed by the second transportation-based analysis processor 245 includes receiving one or more previous information sets 15 for the first user 10 (each referred to herein as a “previous first user information set”, “previous candidate first user information set”, or the like) and/or one or more previous information sets 15 for the second user 10 (each referred to herein as a “previous second user information set”, “previous candidate first user information set”, or the like) from the information set processor 210 (and/or the analysis selector 241, the assessment interface 232, other elements of the processor 200, and/or databases 30). Each previous first user information set 15 received by the second transportation-based analysis processor 245 includes a first previous geolocation 11 for the first user 10 and a first previous timestamp 12 for the first previous geolocation 11 (where each first previous timestamp 12 is a timestamp for a time before the first timestamp 12 (in the candidate first user information set 15); and accordingly, each first previous geolocation 11 represents a geolocation for the first user 10 at the first previous timestamp 12). Furthermore, each previous second user information set 15 includes a second previous geolocation 11 for the second user 10 and a second previous timestamp 12 for the second previous geolocation 11 (where each second previous timestamp 12 is a timestamp for a time before the second timestamp 12 (in the candidate second user information set 15); and accordingly, each second previous geolocation 11 represents a geolocation for the second user 10 at the second previous timestamp 12). For example, if the first timestamp 12 (in the candidate first user information set 15) is a timestamp of 10:10:30 am on 2 Feb. 2020, then the first previous timestamps 12 may be timestamps of 10:10:28 am on 2 Feb. 2020, 10:10:26 am on 2 Feb. 2020, 10:10:24 am on 2 Feb. 2020, 10:10:22 am on 2 Feb. 2020, 10:10:20 am on 2 Feb. 2020, and so on. As another example, if the first timestamp 12 is a timestamp of 10:10:30 am on 2 Feb. 2020, then the first previous timestamps 12 may be timestamps of 10:10:29 am on 2 Feb. 2020, 10:10:28 am on 2 Feb. 2020, 10:10:27 am on 2 Feb. 2020, 10:10:26 am on 2 Feb. 2020, 10:10:25 am on 2 Feb. 2020, and so on.

In addition or alternatively to receiving the one or more previous first user information sets 15 and/or the one or more previous second user information sets 15, the second transportation-based separation analysis performed by the second transportation-based analysis processor 245 may also include receiving one or more subsequent information sets 15 for the first user 10 (each referred to herein as a “subsequent first user information set”, “subsequent candidate first user information set”, or the like) and/or one or more subsequent information sets 15 for the second user 10 (each referred to herein as a “subsequent second user information set”, “subsequent candidate second user information set”, or the like) from the information set processor 210 (and/or the analysis selector 241, the assessment interface 232, other elements of the processor 200, and/or databases 30). Each subsequent first user information set 15 includes a first subsequent geolocation 11 for the first user 10 and a first subsequent timestamp 12 for the first subsequent geolocation 11 (where each first subsequent timestamp 12 is a timestamp for a time after the first timestamp 12 (in the candidate first user information set 15); and accordingly, each first subsequent geolocation 11 represents a geolocation for the first user 10 at the first subsequent timestamp 12). Furthermore, each subsequent second user information set 15 includes a second subsequent geolocation 11 for the second user 10 and a second subsequent timestamp 12 for the second subsequent geolocation 11 (where each second subsequent timestamp 12 is a timestamp for a time after the second timestamp 12 (in the candidate second user information set 15); and accordingly, each second subsequent geolocation 11 represents a geolocation for the second user 10 at the second subsequent timestamp 12). For example, if the first timestamp 12 is a timestamp of 10:10:30 am on 2 Feb. 2020, then the first subsequent timestamps 12 may be timestamps of 10:10:32 am on 2 Feb. 2020, 10:10:34 am on 2 Feb. 2020, 10:10:36 am on 2 Feb. 2020, 10:10:38 am on 2 Feb. 2020, 10:10:40 am on 2 Feb. 2020, and so on. As another example, if the first timestamp 12 is a timestamp of 10:10:30 am on 2 Feb. 2020, then the first subsequent timestamps 12 may be timestamps of 10:10:31 am on 2 Feb. 2020, 10:10:32 am on 2 Feb. 2020, 10:10:33 am on 2 Feb. 2020, 10:10:34 am on 2 Feb. 2020, 10:10:35 am on 2 Feb. 2020, and so on.

In situations where a user 10 carries more than one device (e.g., two or more of any of these: a mobile smart phone, another mobile smart phone, a tablet, a smart watch capable of obtaining geolocations, a pair of smart glasses, etc.), an example embodiment is configurable or configured to associate, link, connect, unify, combine, team, etc. the devices together in such a way that the devices cooperate to gather, record, obtain, generate, and/or send information sets 15 (including candidate information sets 15 (e.g., first candidate information set 15, second candidate information set 15, first candidate first user information set 15, second candidate first user information set 15, first candidate second user information set 15, second candidate second user information set 15, etc.), previous information sets 15 (e.g., previous first user information sets 15, previous second user information sets 15, etc.), subsequent information sets 15 (e.g., subsequent first user information sets 15, subsequent second user information sets 15, etc.), etc., as described above and in the present disclosure). For example, in addition or alternatively to having a single user device generate an information set 15 every 2 seconds, a user 10 with two devices (e.g., a mobile phone and a smart watch; or two mobile phones) may cooperate to collectively generate information sets 15 every 2 seconds by having each device share the responsibility of generating information sets 15 (e.g., in an alternating or staggered manner where devices take turns generating information sets 15 every 4 seconds; in a shift or periodic manner where devices take turns generating information sets 15 every 2 seconds for a certain period, such as 1 minute, 5 minutes, 1 hour, etc.; etc.). The processor 200 is then configurable or configured to identify information sets 15 coming from different devices of the same user 10, in which case all of the information sets 15 are then stored in databases 30 and/or provided for further processing by elements of the processor 200 (as described in the present disclosure) for the user 10 (e.g., based on the user 10 and not based on the device; and/or as if the information sets 15 were generated by one device). Example embodiments may configure the two or more devices to cooperate together in generating the information sets 15 based on one or more considerations. For example, the two or more devices may cooperate in generating information sets 15 only when the devices have been confirmed, by one or more of the devices and/or the processor 200, to be “together” (e.g., to avoid situations where the user 10 carries his/her mobile smart phone but leave his/her iPad in his/her car, in which case the devices are not together). Such confirmations that the devices are “together” (i.e., devices are with the user 10) may be performed in one or more ways, including, but not limited to, implementing a “handshake” or communication between the devices (e.g., via Bluetooth signals, WiFi signals, NFC signals, etc.); comparing recent and/or current information sets 15 generated by each device to determine whether the geolocations 11 are consistent with the devices being together with the user 10; and/or comparing information from other sources and/or applications (e.g., a fitness application installed on the devices; an application that tracks geolocation, such as a map application, installed on the devices; in which case, to protect privacy of users, such a comparison may compare the information from such applications and return a comparison outcome only (e.g., yes or no) without using the information for any other purposes). It is recognized in the present disclosure that having user devices cooperate in generating information sets 15 may enable distributing of processing among more than one device, reducing of processing requirements per device, reducing of power consumption per device, reducing of communication requirements (sending information sets 15 to the processor 200) per device, etc.

After receiving the first candidate contact instance 16 (including the candidate first user information set 15 (for the first user 10, which includes the first geolocation 11 for the first user 10 and the first timestamp 12 for the first geolocation 11) and the candidate second user information set 15 (for the second user 10, which includes the second geolocation 11 for the second user 10 and the second timestamp 12 for the second geolocation 11)), the one or more previous first user information sets 15, the one or more previous second user information sets 15, the one or more subsequent first user information sets 15, and/or the one or more subsequent second user information sets 15, the second transportation-based separation analysis performed by the second transportation-based analysis processor 245 further includes deriving the first different transport mode likelihood for the first candidate contact instance 16 (i.e., a likelihood that the first user 10 and the second user 10 were not traveling using the same transport mode during the first contact instance 16). The first different transport mode likelihood for the first candidate contact instance 16 is derived by first deriving a travel speed of the first user 10. The travel speed of the first user 10 is derived based on two or more information sets 15 for the first user 10. For example, the travel speed of the first user 10 may be derived based on two or more previous first user information sets 15 prior to the first timestamp 12. As another example, the travel speed of the first user 10 may be derived based on the candidate first user information set 15 and one or more previous first user information sets 15. As another example, the travel speed of the first user 10 may be derived based on two or more subsequent first user information sets 15. As another example, the travel speed of the first user 10 may be derived based on the candidate first user information set 15 and one or more subsequent first user information sets 15. As another example, the travel speed of the first user 10 may be derived based on one or more previous first user information sets 15 and one or more subsequent first user information sets 15. As another example, the travel speed of the first user 10 may be derived based on the candidate first user information set 15, one or more previous first user information sets 15, and one or more subsequent first user information sets 15. For each of these examples, the travel speed of the first user 10 can be derived by determining a distance travelled (e.g., distance between geolocations 11) divided by the time taken to travel (e.g., time between timestamps 12). As a more specific example, the second transportation-based analysis processor 245 may receive a previous first user information set 15 having a first previous geolocation 11 and a first previous timestamp 12. The second transportation-based analysis processor 245 may also receive a candidate first user information set 15 having a first geolocation 11 and a first timestamp 12. To derive a travel speed for the first user 10, the second transportation-based analysis processor 245 first determines a distance between the first previous geolocation 11 and the first geolocation 11, followed by a time between the first previous timestamp 12 and the first timestamp 12, followed by dividing the distance by the time.

The second transportation-based analysis processor 245 then derives a travel speed of the second user 10. The travel speed of the second user 10 is derived based on two or more information sets 15 of the second user 10. For example, the travel speed of the second user 10 may be derived based on two or more previous second user information sets 15 prior to the second timestamp 12. As another example, the travel speed of the second user 10 may be derived based on the candidate second user information set 15 and one or more previous second user information sets 15. As another example, the travel speed of the second user 10 may be derived based on two or more subsequent second user information sets 15. As another example, the travel speed of the second user 10 may be derived based on the candidate second user information set 15 and one or more subsequent second user information sets 15. As another example, the travel speed of the second user 10 may be derived based on one or more previous second user information sets 15 and one or more subsequent second user information sets 15. As another example, the travel speed of the second user 10 may be derived based on the candidate second user information set 15, one or more previous second user information sets 15, and one or more subsequent second user information sets 15. For each of these examples, the travel speed of the second user 10 can be derived by determining a distance travelled (e.g., distance between geolocations 11) divided by the time taken to travel (e.g., time between timestamps 12). As a more specific example, the second transportation-based analysis processor 245 may receive a previous second user information set 15 having a second previous geolocation 11 and a second previous timestamp 12. The second transportation-based analysis processor 245 may also receive a candidate second user information set 15 having a second geolocation 11 and a second timestamp 12. To derive a travel speed for the second user 10, the second transportation-based analysis processor 245 first determines a distance between the second previous geolocation 11 and the second geolocation 11, followed by a time between the second previous timestamp 12 and the second timestamp 12, followed by dividing the distance by the time. In an example embodiment, the previous second user information set 15 and the candidate second user information set 15 should be selected for the same or as close as possible to the first previous timestamp 12 and the first timestamp 12 used in deriving the travel speed for the first user 10.

The second transportation-based analysis processor 245 then derives, based on a comparison of the travel speed of the first user 10 and the travel speed of the second user 10, a likelihood that the first user 10 and second user 10 were not traveling using the same transport mode (i.e., the first different transport mode likelihood). In this regard, if the travel speed of the first user 10 is the same as the travel speed of the second user 10, then the first different transport mode likelihood will be low. If, on the other hand, the travel speed of the first user 10 is significantly different from the travel speed of the second user 10, then the first different transport mode likelihood will be high.

FIG. 7C illustrates some examples of geolocations 11 of the first user 10 (which may be first geolocations 11, first previous geolocations 11, and/or first subsequent geolocations 11 of the first user 11; each of which may be referred to as “first geolocations” 11 for the examples of FIG. 7C) and geolocations 11 of the second user 10 (which may be second geolocations 11, second previous geolocations 11, and/or second subsequent geolocations 11; each of which may be referred to as “second geolocations” 11 for the examples of FIG. 7C). In a first example, first geolocation 10c1 (i.e., for the first user 10) and second geolocation 10d1 (i.e., for the second user 10) have the same timestamps 12. Similarly, first geolocation 10c2 (i.e., for the first user 10) and second geolocation 10d2 (i.e., for the second user 10) have the same timestamps 12; first geolocation 10c3 (i.e., for the first user 10) and second geolocation 10d3 (i.e., for the second user 10) have the same timestamps 12; first geolocation 10c4 (i.e., for the first user 10) and second geolocation 10d4 (i.e., for the second user 10) have the same timestamps 12; and first geolocation 10c5 (i.e., for the first user 10) and second geolocation 10d5 (i.e., for the second user 10) have the same timestamps 12. As the distance between any of the two geolocations of the first user 10 is about the same as the distance between any two corresponding geolocations of the second user 10, the second transportation-based analysis processor 245 will derive the travel speed for the first user 10 to be about the same as the travel speed for the second user. In this first example, the first different transport mode likelihood will be very low, the non-contact score for the first contact instance 16 will be very low, and the risk or likelihood of the second user being physically exposed to the first user (or vice versa) will be very high. In a second example, first geolocation 10g2 (i.e., for the first user 10) and second geolocation 10h2 (i.e., for the second user 10) have the same timestamps 12. Similarly, first previous geolocation 10g1 (i.e., for the first user 10) and second previous geolocation 10h1 (i.e., for the second user 10) have the same timestamps 12; first subsequent geolocation 10g3 (i.e., for the first user 10) and second subsequent geolocation 10h3 (i.e., for the second user 10) have the same timestamps 12; and first subsequent geolocation 10g4 (i.e., for the first user 10) and second subsequent geolocation 10h4 (i.e., for the second user 10) have the same timestamps 12. In this second example, the travel speed for the first user 10 will be significantly different from the travel speed for the second user 10. In this second example, the first different transport mode likelihood will be very high, the non-contact score for the first contact instance 16 will be very high, and the risk or likelihood of the second user being physically exposed to the first user (or vice versa) will be very low. In a third example, first geolocation 10a1 (i.e., for the first user 10) and second geolocation 10b1 (i.e., for the second user 10) have the same timestamps 12. Similarly, first subsequent geolocation 10a2 (i.e., for the first user 10) and second subsequent geolocation 10b2 (i.e., for the second user 10) have the same timestamps 12; first subsequent geolocation 10a3 (i.e., for the first user 10) and second subsequent geolocation 10b3 (i.e., for the second user 10) have the same timestamps 12; first subsequent geolocation 10b4 (i.e., for the first user 10) and second subsequent geolocation 10b4 (i.e., for the second user 10) have the same timestamps 12; and first subsequent geolocation 10b5 (i.e., for the first user 10) and second subsequent geolocation 10b5 (i.e., for the second user 10) have the same timestamps 12. In this third example, the travel speed for the first user 10 will be significantly different from the travel speed for the second user 10 (which will be zero since the second user 10 is stationary or not moving). In this third example, the first different transport mode likelihood will be very high, the non-contact score for the first contact instance 16 will be very high, and the risk or likelihood of the second user being physically exposed to the first user (or vice versa) will be very low. In a fourth example, first geolocation 10e1 (i.e., for the first user 10) and second geolocation 10f1 (i.e., for the second user 10) have the same timestamps 12. Similarly, first geolocation 10e2 (i.e., for the first user 10) and second geolocation 10f2 (i.e., for the second user 10) have the same timestamps 12; first geolocation 10e3 (i.e., for the first user 10) and second geolocation 10f3 (i.e., for the second user 10) have the same timestamps 12; first geolocation 10e4 (i.e., for the first user 10) and second geolocation 10f4 (i.e., for the second user 10) have the same timestamps 12; and first geolocation 10e5 (i.e., for the first user 10) and second geolocation 10f5 (i.e., for the second user 10) have the same timestamps 12. As the distance between any of the two geolocations of the first user 10 is about the same as the distance between any two corresponding geolocations of the second user 10, the second transportation-based analysis processor 245 will derive the travel speed for the first user 10 to be about the same as the travel speed for the second user. In this fourth example, the first different transport mode likelihood will be very low, the non-contact score for the first contact instance 16 will be very low, and the risk or likelihood of the second user being physically exposed to the first user (or vice versa) will be very high. However, when the first different transport mode likelihood is combined and/or considered with the first physical barrier likelihood and/or the second physical barrier likelihood, the non-contact score for the first contact instance 16 for the fourth example will be adjusted to be very high despite the first different transport mode likelihood being very low. Furthermore, the risk or likelihood of the second user being physically exposed to the first user (or vice versa) will be very low despite the first different transport mode likelihood being very low. It is recognized in this example that the weighting, value, and/or relevance of the results of any given analysis by the non-contact processor 240 may vary and be based on the results of another analysis by the non-contact processor 240.

After the first different transport mode likelihood is derived/generated, the second transportation-based analysis processor 245 is configurable or configured to obtain a threshold transport mode value from the threshold transport mode generator 206c (e.g., via the main threshold processor 202′). The threshold transport mode value may be dynamically generated by the threshold transport mode generator 206c based on one or more considerations including, but not limited to, the travel speed of the first user 10, the travel speed of the second user 10, the difference between the travel speed of the first user 10 and travel speed of the second user 10, one or more aspects of the first candidate contact instance 16, etc. Alternatively or in addition, the threshold transport mode value may be a default or predetermined threshold value (or selected from one or more default and/or predetermined threshold values). The second transportation-based analysis processor 245 is then configurable or configured to compare the first different transport mode likelihood with the threshold transport mode value.

In an example embodiment, the second transportation-based analysis processor 245 is then configurable or configured to provide the first different transport mode likelihood for the first candidate contact instance 16 to the non-contact score generator 248 for use in deriving the non-contact score for the first candidate contact instance 16 (e.g., along with one or more results from other elements of the non-contact processor 240, including the layout analysis processor 242, the map analysis processor 243, the first transportation-based analysis processor 244, the third transportation-based analysis processor 246, and/or the different levels analysis processor 247). In example embodiments, the second transportation-based analysis processor 245 provides the first different transport mode likelihood to the non-contact score generator 248 based on a comparison of the first different transport mode likelihood with the threshold transport mode value. For example, the second transportation-based analysis processor 245 provides the first different transport mode likelihood to the non-contact score generator 248 when the first different transport mode likelihood is greater than or equal to the threshold transport mode value.

vi. The Third Transportation-Based Analysis Processor (e.g., Element 246).

As illustrated in FIG. 6, an example embodiment of the non-contact processor 240 includes one or more third transportation-based analysis processors (e.g., third transportation-based analysis processor 246). The third transportation-based analysis processor 246 is configurable or configured to receive candidate contact instances 16 and other information from the analysis selector 241 (and/or the assessment interface 232 and/or other elements of the processor 200). Each candidate contact instance 16 received by the third transportation-based analysis processor 246 includes a candidate information set 15 (for a user 10, which includes a geolocation 11 and a timestamp 12 for the geolocation 11) and another candidate information set 15 (for another user 10, which includes a geolocation 11 and a timestamp 12 for the geolocation 11). For example, the third transportation-based analysis processor 246 may receive a first candidate contact instance 16 having a candidate first user information set 15 (for a first user 10, which includes a first geolocation 11 for the first user 10 and a first timestamp 12 for the first geolocation 11) and a candidate second user information set 15 (for a second user 10, which includes a second geolocation 11 for the second user 10 and a second timestamp 12 for the second geolocation 11).

For each first candidate contact instance 16, the third transportation-based analysis processor 246 is configurable or configured to derive/generate a likelihood that a user 10 (e.g., the first user 10) and another user 10 (e.g., the second user 10) were not traveling using the same transport mode (e.g., not traveling using a common transport mode; traveling using different transport modes; one of the users 10 traveling using one transport mode and the other user 10 not traveling (stationary); etc.) (such likelihood referred to herein as the “second different transport mode likelihood”).

The third transportation-based analysis processor 246 is configurable or configured to derive the second different transport mode likelihood by performing, among other things, an example embodiment of a third transportation-based separation analysis (or third transportation-based analysis).

In an example embodiment, the third transportation-based separation analysis performed by the third transportation-based analysis processor 246 includes receiving one or more previous first user information sets 15 for the first user 10 and/or one or more previous second user information sets 15 for the second user 10 from the information set processor 210 (and/or the analysis selector 241, the assessment interface 232, other elements of the processor 200, and/or databases 30). Each previous first user information set 15 received by the third transportation-based analysis processor 246 includes a first previous geolocation 11 for the first user 10 and a first previous timestamp 12 for the first previous geolocation 11 (where each first previous timestamp 12 is a timestamp for a time before the first timestamp 12 (in the candidate first user information set 15); and accordingly, each first previous geolocation 11 represents a geolocation for the first user 10 at the first previous timestamp 12). Furthermore, each previous second user information set 15 includes a second previous geolocation 11 for the second user 10 and a second previous timestamp 12 for the second previous geolocation 11 (where each second previous timestamp 12 is a timestamp for a time before the second timestamp 12 (in the candidate second user information set 15); and accordingly, each second previous geolocation 11 represents a geolocation for the second user 10 at the second previous timestamp 12). For example, if the first timestamp 12 (in the candidate first user information set 15) is a timestamp of 10:10:30 am on 2 Feb. 2020, then the first previous timestamps 12 may be timestamps of 10:10:27 am on 2 Feb. 2020, 10:10:24 am on 2 Feb. 2020, 10:10:21 am on 2 Feb. 2020, 10:10:18 am on 2 Feb. 2020, 10:15:20 am on 2 Feb. 2020, and so on. As another example, if the first timestamp 12 is a timestamp of 10:10:30 am on 2 Feb. 2020, then the first previous timestamps 12 may be timestamps of 10:10:26 am on 2 Feb. 2020, 10:10:22 am on 2 Feb. 2020, 10:10:18 am on 2 Feb. 2020, 10:10:14 am on 2 Feb. 2020, 10:10:10 am on 2 Feb. 2020, and so on.

In addition or alternatively to receiving the one or more previous first user information sets 15 and/or the one or more previous second user information sets 15, the third transportation-based separation analysis performed by the third transportation-based analysis processor 246 may also include receiving one or more subsequent information sets 15 for the first user 10 and/or one or more subsequent information sets 15 for the second user 10 from the information set processor 210 (and/or the analysis selector 241, the assessment interface 232, other elements of the processor 200, and/or databases 30). Each subsequent first user information set 15 includes a first subsequent geolocation 11 for the first user 10 and a first subsequent timestamp 12 for the first subsequent geolocation 11 (where each first subsequent timestamp 12 is a timestamp for a time after the first timestamp 12 (in the candidate first user information set 15); and accordingly, each first subsequent geolocation 11 represents a geolocation for the first user 10 at the first subsequent timestamp 12). Furthermore, each subsequent second user information set 15 includes a second subsequent geolocation 11 for the second user 10 and a second subsequent timestamp 12 for the second subsequent geolocation 11 (where each second subsequent timestamp 12 is a timestamp for a time after the second timestamp 12 (in the candidate second user information set 15); and accordingly, each second subsequent geolocation 11 represents a geolocation for the second user 10 at the second subsequent timestamp 12). For example, if the first timestamp 12 is a timestamp of 10:10:30 am on 2 Feb. 2020, then the first subsequent timestamps 12 may be timestamps of 10:10:31.5 am on 2 Feb. 2020, 10:10:33 am on 2 Feb. 2020, 10:10:34.5 am on 2 Feb. 2020, 10:10:36 am on 2 Feb. 2020, 10:10:37.5 am on 2 Feb. 2020, and so on. As another example, if the first timestamp 12 is a timestamp of 10:10:30 am on 2 Feb. 2020, then the first subsequent timestamps 12 may be timestamps of 10:10:31 am on 2 Feb. 2020, 10:10:32 am on 2 Feb. 2020, 10:10:33 am on 2 Feb. 2020, 10:10:34 am on 2 Feb. 2020, 10:10:35 am on 2 Feb. 2020, and so on.

In situations where a user 10 carries more than one device (e.g., two or more of any of these: a mobile smart phone, another mobile smart phone, a tablet, a smart watch capable of obtaining geolocations, a pair of smart glasses, etc.), an example embodiment is configurable or configured to associate, link, connect, unify, combine, team, etc. the devices together in such a way that the devices cooperate to gather, record, obtain, generate, and/or send information sets 15 (including candidate information sets 15 (e.g., first candidate information set 15, second candidate information set 15, first candidate first user information set 15, second candidate first user information set 15, first candidate second user information set 15, second candidate second user information set 15, etc.), previous information sets 15 (e.g., previous first user information sets 15, previous second user information sets 15, etc.), subsequent information sets 15 (e.g., subsequent first user information sets 15, subsequent second user information sets 15, etc.), etc., as described above and in the present disclosure).

After receiving the first candidate contact instance 16 (including the candidate first user information set 15 (for the first user 10, which includes the first geolocation 11 for the first user 10 and the first timestamp 12 for the first geolocation 11) and the candidate second user information set 15 (for the second user 10, which includes the second geolocation 11 for the second user 10 and the second timestamp 12 for the second geolocation 11)), the one or more previous first user information sets 15, the one or more previous second user information sets 15, the one or more subsequent first user information sets 15, and/or the one or more subsequent second user information sets 15, the third transportation-based separation analysis performed by the third transportation-based analysis processor 246 further includes deriving the second different transport mode likelihood for the first candidate contact instance 16 (i.e., a likelihood that the first user 10 and the second user 10 were not traveling using the same transport mode during the first contact instance 16). The second different transport mode likelihood for the first candidate contact instance 16 is derived by first deriving a travel direction of the first user 10. The travel direction of the first user 10 is derived based on two or more geolocations 11 for the first user 10. For example, the travel direction of the first user 10 may be derived based on two or more previous first user information sets 15 prior to the first timestamp 12. As another example, the travel direction of the first user 10 may be derived based on the candidate first user information set 15 and one or more previous first user information sets 15. As another example, the travel direction of the first user 10 may be derived based on two or more subsequent first user information sets 15. As another example, the travel direction of the first user 10 may be derived based on the candidate first user information set 15 and one or more subsequent first user information sets 15. As another example, the travel direction of the first user 10 may be derived based on one or more previous first user information sets 15 and one or more subsequent first user information sets 15. As another example, the travel direction of the first user 10 may be derived based on the candidate first user information set 15, one or more previous first user information sets 15, and one or more subsequent first user information sets 15. As an example of deriving a travel direction based on previous first user information sets 15, the travel direction of the first user 10 can be derived by assessing a direction of a line drawn from a first previous geolocation 11 of a previous first user information set 15 (e.g., with a first previous timestamp 12 of t1) to another first previous geolocation 11 of another previous first user information set 15 (e.g., with a first previous timestamp 12 of t1+1). As an example of deriving a travel direction based on a previous first user information set 15 and the candidate first user information set 15, the travel direction of the first user 10 can be derived by assessing a direction of a line drawn from a first previous geolocation 11 of a previous first user information set 15 to the first geolocation 11 of the candidate first user information set 15. As an example of deriving a travel direction based on the candidate first user information set 15 and a subsequent first user information set 15, the travel direction of the first user 10 can be derived by assessing a direction of a line drawn from the first geolocation 11 of the candidate first user information set 15 to a first subsequent geolocation 11 of a subsequent first user information set 15. As an example of deriving a travel direction based on subsequent first user information sets 15, the travel direction of the first user 10 can be derived by assessing a direction of a line drawn from a first subsequent geolocation 11 of a subsequent first user information set 15 (e.g., with a first subsequent timestamp 12 of t1) to another first subsequent geolocation 11 of another subsequent first user information set 15 (e.g., with a first subsequent timestamp 12 of t1+1). As an example of deriving a travel direction based on a previous first user information set 15 and a subsequent first user information set 15, the travel direction of the first user 10 can be derived by assessing a direction of a line drawn from a first previous geolocation 11 of a previous first user information set 15 to a first subsequent geolocation 11 of a subsequent first user information set 15.

The third transportation-based analysis processor 246 then derives a travel direction of the second user 10. The travel direction of the second user 10 is derived based on two or more geolocations 11 for the second user 10. For example, the travel direction of the second user 10 may be derived based on two or more previous second user information sets 15 prior to the second timestamp 12. As another example, the travel direction of the second user 10 may be derived based on the candidate second user information set 15 and one or more previous second user information sets 15. As another example, the travel direction of the second user 10 may be derived based on two or more subsequent second user information sets 15. As another example, the travel direction of the second user 10 may be derived based on the candidate second user information set 15 and one or more subsequent second user information sets 15. As another example, the travel direction of the second user 10 may be derived based on one or more previous second user information sets 15 and one or more subsequent second user information sets 15. As another example, the travel direction of the second user 10 may be derived based on the candidate second user information set 15, one or more previous second user information sets 15, and one or more subsequent second user information sets 15. As an example of deriving a travel direction based on previous second user information sets 15, the travel direction of the second user 10 can be derived by assessing a direction of a line drawn from a second previous geolocation 11 of a previous second user information set 15 (e.g., with a second previous timestamp 12 of t1) to another second previous geolocation 11 of another previous second user information set 15 (e.g., with a second previous timestamp 12 of t1+1). As an example of deriving a travel direction based on a previous second user information set 15 and the candidate second user information set 15, the travel direction of the second user 10 can be derived by assessing a direction of a line drawn from a second previous geolocation 11 of a previous second user information set 15 to the second geolocation 11 of the candidate second user information set 15. As an example of deriving a travel direction based on the candidate second user information set 15 and a subsequent second user information set 15, the travel direction of the second user 10 can be derived by assessing a direction of a line drawn from the second geolocation 11 of the candidate second user information set 15 to a second subsequent geolocation 11 of a subsequent second user information set 15. As an example of deriving a travel direction based on subsequent second user information sets 15, the travel direction of the second user 10 can be derived by assessing a direction of a line drawn from a second subsequent geolocation 11 of a subsequent second user information set 15 (e.g., with a second subsequent timestamp 12 of t1) to another second subsequent geolocation 11 of another subsequent second user information set 15 (e.g., with a second subsequent timestamp 12 of t1+1). As an example of deriving a travel direction based on a previous second user information set 15 and a subsequent second user information set 15, the travel direction of the second user 10 can be derived by assessing a direction of a line drawn from a second previous geolocation 11 of a previous second user information set 15 to a second subsequent geolocation 11 of a subsequent second user information set 15.

The third transportation-based analysis processor 246 then derives, based on a comparison of the travel direction of the first user 10 and the travel direction of the second user 10, a likelihood that the first user 10 and second user 10 were not traveling using the same transport mode (i.e., the second different transport mode likelihood). In this regard, if the travel direction of the first user 10 is the same as the travel direction of the second user 10, then the second different transport mode likelihood will be low. If, on the other hand, the travel direction of the first user 10 is significantly different from the travel direction of the second user 10, then the second different transport mode likelihood will be high.

FIG. 7D illustrates some examples of geolocations 11 of the first user 10 (which may be first geolocations 11, first previous geolocations 11, and/or first subsequent geolocations 11 of the first user 11; each of which may be referred to as “first geolocations” 11 for the examples of FIG. 7D) and geolocations 11 of the second user 10 (which may be second geolocations 11, second previous geolocations 11, and/or second subsequent geolocations 11; each of which may be referred to as “second geolocations” 11 for the examples of FIG. 7D). In a first example, first geolocation 10a1 (i.e., for the first user 10) and second geolocation 10b1 (i.e., for the second user 10) have the same timestamps 12. Similarly, first geolocation 10a2 (i.e., for the first user 10) and second geolocation 10b2 (i.e., for the second user 10) have the same timestamps 12; first geolocation 10a3 (i.e., for the first user 10) and second geolocation 10b3 (i.e., for the second user 10) have the same timestamps 12; first geolocation 10a4 (i.e., for the first user 10) and second geolocation 10b4 (i.e., for the second user 10) have the same timestamps 12; and first geolocation 10a5 (i.e., for the first user 10) and second geolocation 10b5 (i.e., for the second user 10) have the same timestamps 12. As a direction of a line drawn between any of the two geolocations of the first user 10 is about the same direction as a line drawn between any two corresponding geolocations of the second user 10 (i.e., with same timestamps 12), the third transportation-based analysis processor 246 will derive the travel direction for the first user 10 to be about the same as the travel direction for the second user 10. In this first example, the second different transport mode likelihood will be very low, the non-contact score for the first contact instance 16 will be very low, and the risk or likelihood of the second user being physically exposed to the first user (or vice versa) will be very high. In a second example, first geolocation 10c1 (i.e., for the first user 10) and second geolocation 10d1 (i.e., for the second user 10) have the same timestamps 12. Similarly, first geolocation 10c2 (i.e., for the first user 10) and second geolocation 10d2 (i.e., for the second user 10) have the same timestamps 12; first geolocation 10c3 (i.e., for the first user 10) and second geolocation 10d3 (i.e., for the second user 10) have the same timestamps 12; first geolocation 10c4 (i.e., for the first user 10) and second geolocation 10d4 (i.e., for the second user 10) have the same timestamps 12; and first geolocation 10c5 (i.e., for the first user 10) and second geolocation 10d5 (i.e., for the second user 10) have the same timestamps 12. As a direction of a line drawn between any of the two geolocations of the first user 10 is in a different direction as a line drawn between any two corresponding geolocations of the second user 10 (i.e., with same timestamps 12), the third transportation-based analysis processor 246 will derive the travel direction for the first user 10 to be different from the travel direction for the second user 10. In this second example, the second different transport mode likelihood will be high, the non-contact score for the first contact instance 16 will be high, and the risk or likelihood of the second user being physically exposed to the first user (or vice versa) will be very low. In a third example, first geolocation 10e3 (i.e., for the first user 10) and second geolocation 10f3 (i.e., for the second user 10) have the same timestamps 12. Similarly, first subsequent geolocation 10e4 (i.e., for the first user 10) and second subsequent geolocation 10f4 (i.e., for the second user 10) have the same timestamps 12; first subsequent geolocation 10e5 (i.e., for the first user 10) and second subsequent geolocation 10f5 (i.e., for the second user 10) have the same timestamps 12; first previous geolocation 10e2 (i.e., for the first user 10) and second previous geolocation 10e2 (i.e., for the second user 10) have the same timestamps 12; and first previous geolocation 10e1 (i.e., for the first user 10) and second previous geolocation 10e1 (i.e., for the second user 10) have the same timestamps 12. As a direction of a line drawn between any of the two geolocations of the first user 10 is in a different direction (i.e., directly opposite direction) as a line drawn between any two corresponding geolocations of the second user 10 (i.e., with same timestamps 12), the third transportation-based analysis processor 246 will derive the travel direction for the first user 10 to be different from the travel direction for the second user 10. In this third example, the different transport mode likelihood will be high, the non-contact score for the first contact instance 16 will be high, and the risk or likelihood of the second user being physically exposed to the first user (or vice versa) will be low.

After the second different transport mode likelihood is derived/generated, the third transportation-based analysis processor 246 is configurable or configured to obtain a second threshold transport mode value from the threshold transport mode generator 206c (e.g., via the main threshold processor 202′). The second threshold transport mode value may be dynamically generated by the threshold transport mode generator 206c based on one or more considerations including, but not limited to, the travel direction of the first user 10, the travel direction of the second user 10, the difference between the travel direction of the first user 10 and travel direction of the second user 10 (e.g., difference between the angles of the directions); or angle between the two directions), one or more aspects of the first candidate contact instance 16, etc. Alternatively or in addition, the second threshold transport mode value may be a default or predetermined threshold value (or selected from one or more default and/or predetermined threshold values). The third transportation-based analysis processor 246 is then configurable or configured to compare the second different transport mode likelihood with the second threshold transport mode value.

In an example embodiment, the third transportation-based analysis processor 246 is then configurable or configured to provide the second different transport mode likelihood for the first candidate contact instance 16 to the non-contact score generator 248 for use in deriving the non-contact score for the first candidate contact instance 16 (e.g., along with one or more results from other elements of the non-contact processor 240, including the layout analysis processor 242, the map analysis processor 243, the first transportation-based analysis processor 244, the third transportation-based analysis processor 246, and/or the different levels analysis processor 247). In example embodiments, the third transportation-based analysis processor 246 provides the second different transport mode likelihood to the non-contact score generator 248 based on a comparison of the second different transport mode likelihood with the second threshold transport mode value. For example, the third transportation-based analysis processor 246 provides the second different transport mode likelihood to the non-contact score generator 248 when the second different transport mode likelihood is greater than or equal to the second threshold transport mode value.

vii. The Different Levels Analysis Processor (e.g., Element 247).

As illustrated in FIG. 6, an example embodiment of the non-contact processor 240 includes one or more different levels analysis processors (e.g., different levels analysis processor 247 or different level analysis processor 247). The different levels analysis processor 247 is configurable or configured to receive candidate contact instances 16 and other information from the analysis selector 241 (and/or the assessment interface 232 and/or other elements of the processor 200). Each candidate contact instance 16 received by the different levels analysis processor 247 includes a candidate information set 15 (for a user 10, which includes a geolocation 11 and a timestamp 12 for the geolocation 11) and another candidate information set 15 (for another user 10, which includes a geolocation 11 and a timestamp 12 for the geolocation 11). For example, the different levels analysis processor 247 may receive a first candidate contact instance 16 having a candidate first user information set 15 (for a first user 10, which includes a first geolocation 11 for the first user 10 and a first timestamp 12 for the first geolocation 11) and a candidate second user information set 15 (for a second user 10, which includes a second geolocation 11 for the second user 10 and a second timestamp 12 for the second geolocation 11).

For each first candidate contact instance 16, the different levels analysis processor 247 is configurable or configured to derive/generate a likelihood that the first user 10 and the second user 10 were on different levels (or floors) during the first contact instance 16 (referred to herein as the “different level likelihood” or “different levels likelihood”). The deriving/generating of the different level likelihood includes, but is not limited to, deriving/generating one or more of the following (each of which also referred to herein as a component or consideration for the different level likelihood): (1) a likelihood of a physical barrier between a first previous geolocation 11 (i.e., a geolocation for the first user 10 with a timestamp 12 prior to or before the first timestamp 12) and another first previous geolocation 11 (e.g., a geolocation for the first user 10 with a timestamp 12 prior to or before the timestamp 12 of the first previous geolocation 11) (referred to herein as the “first first user previous different level likelihood”); and/or (2) a likelihood of a physical barrier between a first previous geolocation 11 (i.e., a geolocation for the first user 10 with a timestamp 12 prior to or before the first timestamp 12) and the first geolocation 11 (referred to herein as the “second first user previous different level likelihood”); and/or (3) a likelihood of a physical barrier between the first geolocation 11 and a first subsequent geolocation 11 (i.e., a geolocation for the first user 10 with a timestamp 12 subsequent to or after the first timestamp 12) (referred to herein as the “first first user subsequent different level likelihood”); and/or (4) a likelihood of a physical barrier between a first subsequent geolocation 11 (i.e., a geolocation for the first user 10 with a timestamp 12 subsequent to or after the first timestamp 12) and another first subsequent geolocation (e.g., a geolocation for the first user 10 with a timestamp 12 subsequent to or after the timestamp 12 of the first subsequent geolocation 11) (referred to herein as the “second first user subsequent different level likelihood”); and/or (5) a likelihood of a physical barrier between a second previous geolocation 11 (i.e., a geolocation for the second user 10 with a timestamp 12 prior to or before the second timestamp 12) and another second previous geolocation 11 (e.g., a geolocation for the second user 10 with a timestamp 12 prior to or before the timestamp 12 of the second previous geolocation 11) (referred to herein as the “first second user previous different level likelihood”); and/or (6) a likelihood of a physical barrier between a second previous geolocation 11 (i.e., a geolocation for the second user 10 with a timestamp 12 prior to or before the second timestamp 12) and the second geolocation 11 (referred to herein as the “second second user previous different level likelihood”); and/or (7) a likelihood of a physical barrier between the second geolocation 11 and a second subsequent geolocation 11 (i.e., a geolocation for the second user 10 with a timestamp 12 subsequent to or after the second timestamp 12) (referred to herein as the “first second user subsequent different level likelihood”); and/or (8) a likelihood of a physical barrier between a second subsequent geolocation 11 (i.e., a geolocation for the second user 10 with a timestamp 12 subsequent to or after the second timestamp 12) and another second subsequent geolocation (e.g., a geolocation for the second user 10 with a timestamp 12 subsequent to or after the timestamp 12 of the second subsequent geolocation 11) (referred to herein as the “second second user subsequent different level likelihood”). As described above and in the present disclosure, the physical barrier may be and/or include any part of a building, object, fixture, living being or thing, naturally occurring thing, man-made thing, non-man-made thing, etc.; and such a physical barrier, if present during the first candidate contact instance 16 at a physical barrier location that corresponds to any location between the first user 10 (first geolocation 11, one or more first previous geolocations 11, and/or one or more first subsequent geolocations 11) and second user 10 (second geolocation 11, one or more second previous geolocations, and/or one or more second subsequent geolocations 11), would result in a reduced, lowered, and/or diminished likelihood of a physical exposure between the first user 10 and second user 10.

The different levels analysis processor 247 is configurable or configured to derive the different level likelihood, including the first first user previous different level likelihood, the second first user previous different level likelihood, the first first user subsequent different level likelihood, the second first user subsequent different level likelihood, the first second user previous different level likelihood, the second second user previous different level likelihood, the first second user subsequent different level likelihood, and the second second user subsequent different level likelihood, by performing, among other things, an example embodiment of a different level analysis (or different levels analysis).

The different level analysis performed by the different levels analysis processor 247 includes performing a search, using the first geolocation 11 and/or the second geolocation 11 (and/or one or more first previous geolocations 11, one or more first subsequent geolocations 11, one or more second previous geolocations 11, and/or one or more second subsequent geolocations 11), for information regarding the first geolocation 11 and/or second geolocation 11 (and/or one or more first previous geolocations 11, one or more first subsequent geolocations 11, one or more second previous geolocations 11, and/or one or more second subsequent geolocations 11). Such information regarding the first geolocation 11 and/or second geolocation 11 (and/or one or more first previous geolocations 11, one or more first subsequent geolocations 11, one or more second previous geolocations 11, and/or one or more second subsequent geolocations 11) may include, but is not limited to, information pertaining to, related to, surrounding, associated with, attached to, linked to, encompassing, matching, etc. the first geolocation 11 and/or second geolocation 11 (and/or one or more first previous geolocations 11, one or more first subsequent geolocations 11, one or more second previous geolocations 11, and/or one or more second subsequent geolocations 11).

For example, the different level analysis may include searching, based on the first geolocation 11 and/or the second geolocation 11, for one or more of the following that include, cover, and/or occupy an area or space in which the first geolocation 11 and/or second geolocation 11 are located: one or more addresses (e.g., postal or street address, etc.); one or more building types (e.g., single level building or multi-level building, such as a single level residential home (or bungalow), multi-level residential home, single level office building, multi-level office building, single level retail shop, multi-level retail shop, single level restaurant, multi-level restaurant, single level parking lot, multi-level parking lot, not in a building at all, etc.) and/or building names; one or more building intended purposes (e.g., residential, office, retail, restaurant, etc.); one or more business types and/or business names (if applicable); etc. that includes the first geolocation 11 and/or second geolocation. With one or more such information, the different level analysis may then identify whether the first geolocation 11 and/or the second geolocation 11 is/are in a multi-level building, a single level building, or not in a building at all (e.g., walking outdoors). For example, the different level analysis may search for and determine that the first geolocation 11 is located within a particular room of a single level retail store (e.g., an Audemars Piguet boutique store; a Lululemon store; a Guardian pharmacy store; etc.). As another example, the different level analysis may search for and determine that the first geolocation 11 and second geolocation 11 are located in a multi-level office building (e.g., Marina Bay Financial Centre Tower 1, etc.). As another example, the different level analysis may search for and determine that the first geolocation 11 and second geolocation 11 are both located outdoors (e.g., Labrador Park, sidewalk along Sukhumvit Road, etc.).

The different level analysis further includes performing an analysis to determine whether or not the first geolocation 11 and/or the second geolocation 11 are in a multi-level building, or the like (e.g., a building having multiple levels or floors; any other structure other than a building having multiple levels or floors that allow users 10 to walk or travel through each level; an underground structure, such as those in a subway or metro train systems (e.g., each station of an MRT system in Singapore, MTR system in Hong Kong, BTS system in Bangkok, TTC system in Toronto, etc.); etc.). In an example embodiment, the different level analysis may determine the first geolocation 11 and/or the second geolocation 11 is/are in a multi-level building based on a search of a floor layout database or platform 30 (as described in the present disclosure). In such an example, if a geographical region covering the first and second geolocations 11 is found to have more than one floor layout based on such a search, then the different level analysis may determine that the first geolocation 11 and/or the second geolocation 11 is/are in a multi-level building. If the different level analysis determines that the first geolocation 11 and second geolocation 11 are not in a multi-level building (e.g., a single level retail shop; outdoors; etc.), then the different level likelihood will be low or zero. If, on the other hand, the different level analysis determines that the first geolocation 11 and second geolocation 11 are in a multi-level building, then the different level likelihood will be high.

After the different level analysis searches for more information regarding the first geolocation 11 and the second geolocation 11 (as described above and in the present disclosure) and determines that the first geolocation 11 and/or second geolocation 11 is/are in a multi-level building, or the like, the different level analysis performs a search, in one or more databases or platforms 30 (e.g., a floor layout database or platform 30, one or more sources on the internet (e.g., an internet-accessible repository of floor layouts), other information sources, etc.), for floor layouts, or the like, of the multi-level building that may cover or include the first and second geolocations 11 (e.g., since, in general, use of geolocation information (e.g., first geolocation 11 and second geolocation 11) alone is presently unable to accurately determine which level the first user 10 and second user 10 are located within a multi-level building). Such a search (if not already performed earlier to determine whether or not the first and second geolocations 11 are in a multi-level building) may include searching for and obtaining a floor layout for some or all of the levels of the multi-level building. Alternatively or in addition, the different levels analysis processor 247 may be configurable or configured to: search, in one or more map databases or platforms 30, for one or more maps that include or cover the first geolocation 11 and the second geolocation 11 (and/or a geographic area or region that includes the first geolocation 11 and the second geolocation 11); search, in one or more floor layout databases or platforms 30, for corresponding floor layouts of the multi-level building that include or cover one or more geographic areas or regions in the one or more maps; and overlay, compare, and/or correlate the floor layouts of the multi-level building over or against (as applicable) the one or more map(s) (or vice versa) to determine and/or confirm the locations of the first geolocation 11 and second geolocation 11 on or within each of the floor layouts. Alternatively or in addition, the different levels analysis processor 247 may be configurable or configured to: search, in one or more floor layout databases or platforms 30, for floor layouts of the multi-level building; search, in one or more map databases or platforms 30, for one or more corresponding maps that include or cover the first geolocation 11 and the second geolocation 11 (and/or a geographic area or region that includes the first geolocation 11 and the second geolocation 11); and overlay, compare, and/or correlate the floor layouts of the multi-level building over or against (as applicable) the one or more map(s) (or vice versa) to determine and/or confirm the locations of the first geolocation 11 and second geolocation 11 on or within the floor layout(s). In example embodiments when more than one version, amendment, copy, etc. of a floor layout is found in the searches (e.g., changes to a floor layout), the search may include a consideration of date/time stamps, validity dates, revision/modification/update dates, etc. of the floor layouts in view of the first and second timestamps 12 to ensure appropriate floor layouts are selected.

After obtaining the floor layouts for the multi-level building (e.g., a first floor layout for one floor of the multi-level building and second floor layout for another floor of the multi-level building), the different level analysis performed by the different levels analysis processor 247 further includes identifying, for each obtained floor layout of the multi-level building, a first location on the floor layout. The first location on each floor layout represents the first geolocation 11 of the first user 10 (e.g., location of a user 10 provided on a map, such as Google Maps, Apple Maps, etc.). The different levels analysis processor 247 then identifies, for each floor layout of the multi-level building, a second location on the floor layout. The second location on each floor layout represents the second geolocation 11 of the second user 10.

The different level analysis performed by the different levels analysis processor 247 further includes identifying, for each floor layout of the multi-level building, one or more previous first locations on the floor layout. Each previous first location on each floor layout represents a first previous geolocation 11 of the first user 10. The different levels analysis processor 247 then identifies, for each floor layout of the multi-level building, one or more previous second locations on the floor layout. Each previous second location on each floor layout represents a second previous geolocation 11 of the second user 10.

The different level analysis performed by the different levels analysis processor 247 further includes identifying, for each floor layout of the multi-level building, one or more subsequent first locations on the floor layout. Each subsequent first location on each floor layout represents a first subsequent geolocation 11 of the first user 10. The different levels analysis processor 247 then identifies, for each floor layout of the multi-level building, one or more subsequent second locations on the floor layout. Each subsequent second location on each floor layout represents a second subsequent geolocation 11 of the second user 10.

For each floor layout of the multi-level building, the different level analysis performed by the different levels analysis processor 247 includes deriving/generating, based on a plurality of previous first locations (e.g., for 2-10 previous first locations, or more or less), a likelihood that a physical barrier is present between any two of the previous first locations (i.e., the first first user previous different level likelihood). As described above and in the present disclosure (e.g., for the layout analysis processor 242), a physical barrier may be determined between any two of the previous first locations using, among other things, image recognition to identify lines, shapes, etc. that may correspond to physical barriers on the floor layout (e.g., a wall, window, door, elevator, stairs, etc.), character recognition (e.g., OCR) to identify textual descriptions and/or labels for physical barriers on the floor layout, etc. For example, the different levels analysis processor 247 may include and/or cooperate with one or more forms of artificial intelligence (AI) (e.g., machine learning or deep learning via convolution neural networks for image recognition, etc.) to determine the likelihood that lines, shapes, figures, characters, symbols, etc. found in each floor layout represent and/or refer to physical barriers. If a physical barrier is unlikely to be present between any and all of the previous first locations, then the first first user previous different level likelihood for that particular floor layout is low. If, on the other hand, a physical barrier is likely to be present between any two previous first locations, then the first first user previous different level likelihood for that particular floor layout is high. An overall first first user previous different level likelihood (i.e., for all floor layouts for the multi-level building) is then derived/generated based on the first first user previous different level likelihoods for all the floor layouts for the multi-level building. For example, the overall first first user previous different level likelihood will be: high (when the analysis determines that, for most or all of the floor layouts of the multi-level building, a physical barrier is present (e.g., 60-100%)); medium (when the analysis determined that, for about half of the floor layouts of the multi-level building, a physical barrier is present (e.g., 41-59%)); low (when the analysis determines that, for some of the floor layouts of the multi-level building, a physical barrier is present (e.g., 1-40%)); or undeterminable (when the analysis determines that, for none of the floor layouts of the multi-level building, a physical barrier is present (e.g., 0%)).

Alternatively or in addition, for each floor layout of the multi-level building, the different level analysis performed by the different levels analysis processor 247 may derive/generate, based on the first location and one or more previous first locations (e.g., for 1-10 previous first locations, or more or less), a likelihood that a physical barrier is present between any two such locations (i.e., the second first user previous different level likelihood). As described above and in the present disclosure (e.g., for the layout analysis processor 242), a physical barrier may be determined between any two such locations using, among other things, image recognition to identify lines, shapes, etc. that may correspond to physical barriers on the floor layout (e.g., a wall, window, door, elevator, stairs, etc.), character recognition (e.g., OCR) to identify textual descriptions and/or labels for physical barriers on the floor layout, etc. If a physical barrier is unlikely to be present between any and all such locations, then the second first user previous different level likelihood for that particular floor layout is low. If, on the other hand, a physical barrier is likely to be present between any two such locations, then the second first user previous different level likelihood for that particular floor layout is high. An overall second first user previous different level likelihood (i.e., for all floor layouts for the multi-level building) is then derived/generated based on the second first user previous different level likelihoods for all the floor layouts for the multi-level building. For example, the overall second first user previous different level likelihood will be: high (when the analysis determines that, for most or all of the floor layouts of the multi-level building, a physical barrier is present (e.g., 60-100%)); medium (when the analysis determined that, for about half of the floor layouts of the multi-level building, a physical barrier is present (e.g., 41-59%)); low (when the analysis determines that, for some of the floor layouts of the multi-level building, a physical barrier is present (e.g., 1-40%)); or undeterminable (when the analysis determines that, for none of the floor layouts of the multi-level building, a physical barrier is present (e.g., 0%)).

Alternatively or in addition, for each floor layout of the multi-level building, the different level analysis performed by the different levels analysis processor 247 may derive/generate, based on a plurality of subsequent first locations (e.g., for 2-10 subsequent first locations, or more or less), a likelihood that a physical barrier is present between any two of the subsequent first locations (i.e., the first first user subsequent different level likelihood). As described above and in the present disclosure (e.g., for the layout analysis processor 242), a physical barrier may be determined between any two of the subsequent first locations using, among other things, image recognition to identify lines, shapes, etc. that may correspond to physical barriers on the floor layout (e.g., a wall, window, door, elevator, stairs, etc.), character recognition (e.g., OCR) to identify textual descriptions and/or labels for physical barriers on the floor layout, etc. If a physical barrier is unlikely to be present between any and all of the subsequent first locations, then the first first user subsequent different level likelihood for that particular floor layout is low. If, on the other hand, a physical barrier is likely to be present between any two subsequent first locations, then the first first user subsequent different level likelihood for that particular floor layout is high. An overall first first user subsequent different level likelihood (i.e., for all floor layouts for the multi-level building) is then derived/generated based on the first first user subsequent different level likelihoods for all the floor layouts for the multi-level building. For example, the overall first first user subsequent different level likelihood will be: high (when the analysis determines that, for most or all of the floor layouts of the multi-level building, a physical barrier is present (e.g., 60-100%)); medium (when the analysis determined that, for about half of the floor layouts of the multi-level building, a physical barrier is present (e.g., 41-59%)); low (when the analysis determines that, for some of the floor layouts of the multi-level building, a physical barrier is present (e.g., 1-40%)); or undeterminable (when the analysis determines that, for none of the floor layouts of the multi-level building, a physical barrier is present (e.g., 0%)).

Alternatively or in addition, for each floor layout of the multi-level building, the different level analysis performed by the different levels analysis processor 247 may derive/generate, based on the first location and one or more subsequent first locations (e.g., for 1-10 subsequent first locations, or more or less), a likelihood that a physical barrier is present between any two such locations (i.e., the second first user subsequent different level likelihood). As described above and in the present disclosure (e.g., for the layout analysis processor 242), a physical barrier may be determined between any two such locations using, among other things, image recognition to identify lines, shapes, etc. that may correspond to physical barriers on the floor layout (e.g., a wall, window, door, elevator, stairs, etc.), character recognition (e.g., OCR) to identify textual descriptions and/or labels for physical barriers on the floor layout, etc. If a physical barrier is unlikely to be present between any and all such locations, then the second first user subsequent different level likelihood for that particular floor layout is low. If, on the other hand, a physical barrier is likely to be present between any two such locations, then the second first user subsequent different level likelihood for that particular floor layout is high. An overall second first user subsequent different level likelihood (i.e., for all floor layouts for the multi-level building) is then derived/generated based on the second first user subsequent different level likelihoods for all the floor layouts for the multi-level building. For example, the overall second first user subsequent different level likelihood will be: high (when the analysis determines that, for most or all of the floor layouts of the multi-level building, a physical barrier is present (e.g., 60-100%)); medium (when the analysis determined that, for about half of the floor layouts of the multi-level building, a physical barrier is present (e.g., 41-59%)); low (when the analysis determines that, for some of the floor layouts of the multi-level building, a physical barrier is present (e.g., 1-40%)); or undeterminable (when the analysis determines that, for none of the floor layouts of the multi-level building, a physical barrier is present (e.g., 0%)).

Alternatively or in addition, for each floor layout of the multi-level building, the different level analysis performed by the different levels analysis processor 247 may derive/generate, based on a plurality of previous second locations (e.g., for 2-10 previous second locations, or more or less), a likelihood that a physical barrier is present between any two of the previous second locations (i.e., the first second user previous different level likelihood). As described above and in the present disclosure (e.g., for the layout analysis processor 242), a physical barrier may be determined between any two of the previous second locations using, among other things, image recognition to identify lines, shapes, etc. that may correspond to physical barriers on the floor layout (e.g., a wall, window, door, elevator, stairs, etc.), character recognition (e.g., OCR) to identify textual descriptions and/or labels for physical barriers on the floor layout, etc. If a physical barrier is unlikely to be present between any and all of the previous second locations, then the first second user previous different level likelihood for that particular floor layout is low. If, on the other hand, a physical barrier is likely to be present between any two previous second locations, then the first second user previous different level likelihood for that particular floor layout is high. An overall first second user previous different level likelihood (i.e., for all floor layouts for the multi-level building) is then derived/generated based on the first second user previous different level likelihoods for all the floor layouts for the multi-level building. For example, the overall first second user previous different level likelihood will be: high (when the analysis determines that, for most or all of the floor layouts of the multi-level building, a physical barrier is present (e.g., 60-100%)); medium (when the analysis determined that, for about half of the floor layouts of the multi-level building, a physical barrier is present (e.g., 41-59%)); low (when the analysis determines that, for some of the floor layouts of the multi-level building, a physical barrier is present (e.g., 1-40%)); or undeterminable (when the analysis determines that, for none of the floor layouts of the multi-level building, a physical barrier is present (e.g., 0%)).

Alternatively or in addition, for each floor layout of the multi-level building, the different level analysis performed by the different levels analysis processor 247 may derive/generate, based on the second location and one or more previous second locations (e.g., for 1-10 previous second locations, or more or less), a likelihood that a physical barrier is present between any two such locations (i.e., the second second user previous different level likelihood). As described above and in the present disclosure (e.g., for the layout analysis processor 242), a physical barrier may be determined between any two such locations using, among other things, image recognition to identify lines, shapes, etc. that may correspond to physical barriers on the floor layout (e.g., a wall, window, door, elevator, stairs, etc.), character recognition (e.g., OCR) to identify textual descriptions and/or labels for physical barriers on the floor layout, etc. If a physical barrier is unlikely to be present between any and all such locations, then the second second user previous different level likelihood for that particular floor layout is low. If, on the other hand, a physical barrier is likely to be present between any two such locations, then the second second user previous different level likelihood for that particular floor layout is high. An overall second second user previous different level likelihood (i.e., for all floor layouts for the multi-level building) is then derived/generated based on the second second user previous different level likelihoods for all the floor layouts for the multi-level building. For example, the overall second second user previous different level likelihood will be: high (when the analysis determines that, for most or all of the floor layouts of the multi-level building, a physical barrier is present (e.g., 60-100%)); medium (when the analysis determined that, for about half of the floor layouts of the multi-level building, a physical barrier is present (e.g., 41-59%)); low (when the analysis determines that, for some of the floor layouts of the multi-level building, a physical barrier is present (e.g., 1-40%)); or undeterminable (when the analysis determines that, for none of the floor layouts of the multi-level building, a physical barrier is present (e.g., 0%)).

Alternatively or in addition, for each floor layout of the multi-level building, the different level analysis performed by the different levels analysis processor 247 may derive/generate, based on a plurality of subsequent second locations (e.g., for 2-10 subsequent second locations, or more or less), a likelihood that a physical barrier is present between any two of the subsequent second locations (i.e., the first second user subsequent different level likelihood). As described above and in the present disclosure (e.g., for the layout analysis processor 242), a physical barrier may be determined between any two of the subsequent first locations using, among other things, image recognition to identify lines, shapes, etc. that may correspond to physical barriers on the floor layout (e.g., a wall, window, door, elevator, stairs, etc.), character recognition (e.g., OCR) to identify textual descriptions and/or labels for physical barriers on the floor layout, etc. If a physical barrier is unlikely to be present between any and all of the subsequent second locations, then the first second user subsequent different level likelihood for that particular floor layout is low. If, on the other hand, a physical barrier is likely to be present between any two subsequent second locations, then the first second user subsequent different level likelihood for that particular floor layout is high. An overall first second user subsequent different level likelihood (i.e., for all floor layouts for the multi-level building) is then derived/generated based on the first second user subsequent different level likelihoods for all the floor layouts for the multi-level building. For example, the overall first second user subsequent different level likelihood will be: high (when the analysis determines that, for most or all of the floor layouts of the multi-level building, a physical barrier is present (e.g., 60-100%)); medium (when the analysis determined that, for about half of the floor layouts of the multi-level building, a physical barrier is present (e.g., 41-59%)); low (when the analysis determines that, for some of the floor layouts of the multi-level building, a physical barrier is present (e.g., 1-40%)); or undeterminable (when the analysis determines that, for none of the floor layouts of the multi-level building, a physical barrier is present (e.g., 0%)).

Alternatively or in addition, for each floor layout of the multi-level building, the different level analysis performed by the different levels analysis processor 247 may derive/generate, based on the second location and one or more subsequent second locations (e.g., for 1-10 subsequent second locations, or more or less), a likelihood that a physical barrier is present between any two such locations (i.e., the second second user subsequent different level likelihood). As described above and in the present disclosure (e.g., for the layout analysis processor 242), a physical barrier may be determined between any two such locations using, among other things, image recognition to identify lines, shapes, etc. that may correspond to physical barriers on the floor layout (e.g., a wall, window, door, elevator, stairs, etc.), character recognition (e.g., OCR) to identify textual descriptions and/or labels for physical barriers on the floor layout, etc. If a physical barrier is unlikely to be present between any and all such locations, then the second second user subsequent different level likelihood for that particular floor layout is low. If, on the other hand, a physical barrier is likely to be present between any two such locations, then the second second user subsequent different level likelihood for that particular floor layout is high. An overall second second user subsequent different level likelihood (i.e., for all floor layouts for the multi-level building) is then derived/generated based on the second second user subsequent different level likelihoods for all the floor layouts for the multi-level building. For example, the overall second second user subsequent different level likelihood will be: high (when the analysis determines that, for most or all of the floor layouts of the multi-level building, a physical barrier is present (e.g., 60-100%)); medium (when the analysis determined that, for about half of the floor layouts of the multi-level building, a physical barrier is present (e.g., 41-59%)); low (when the analysis determines that, for some of the floor layouts of the multi-level building, a physical barrier is present (e.g., 1-40%)); or undeterminable (when the analysis determines that, for none of the floor layouts of the multi-level building, a physical barrier is present (e.g., 0%)).

In deriving the different level likelihood, including one or more of the components or considerations of the different level likelihood, the different level analysis processor 247 may assess one or more of the following: a likelihood that the physical barrier identified by such analysis is indeed a physical barrier; a likelihood that the physical barrier identified by such analysis is not located at the physical barrier location (i.e., not at a location between such locations); a likelihood that the floor layout is not an accurate or correct floor layout; a likelihood that the location of the physical barrier, geolocations 11, and/or physical barrier locations are inaccurate and/or not correct.

As an illustrative example, the different level analysis processor 247 may determine, based on a first geolocation 11 and a second geolocation 11 of a first candidate contact instance 16, that the first geolocation 11 and second geolocation are in a multi-level building (e.g., 2 level building), such as based on a search for an address, map(s), and/or floor layout(s), and/or search in other sources 30. The different level analysis processor 247 then obtains a first floor layout (i.e., a floor layout for one of the levels of the multi-level building, such as the floor layout for floor 1F) and a second floor layout (i.e., a floor layout for another one of the levels of the multi-level building, such as the floor layout for floor 2F). FIG. 7E illustrates some examples of first locations (i.e., first geolocation 11 for the first user 10), previous first locations (i.e., first previous geolocations 11 for the first user 10), subsequent first locations (i.e., first subsequent geolocations 11 for the first user 10), second locations (i.e., second geolocation 11 for the second user 10), previous second locations (i.e., second previous geolocations 11 for the second user 10), and subsequent second locations (i.e., second subsequent geolocations 11 for the second user 10) on the first floor layout of the multi-level building, and FIG. 7F illustrates the same first locations, previous first locations, subsequent first locations, second locations, previous second locations, and subsequent second locations on the second floor layout of the multi-level building.

In a first example, a different level analysis is first performed on the first floor layout (e.g., floor 1F) illustrated in FIG. 7E and second floor layout (e.g., floor 2F) illustrated in FIG. 7F for first location 10a3 (i.e., first geolocation 11 for the first user) and second location 10b3 (i.e., second geolocation 11 for the second user). The different level analysis includes deriving a first first user previous different level likelihood (first floor layout) for previous first location 10a1 and previous first location 10a2. In doing so, the different level analysis determines that a physical barrier is very unlikely to be present between the previous first locations 10a1 and 10a2 on the first floor layout and therefore the first first user previous different level likelihood (first floor layout) for previous first locations 10a1 and 10a2 is very low. The different level analysis then derives, based on the second floor layout, a first first user previous different level likelihood (second floor layout) for previous first locations 10a1 and 10a2. In doing so, the different level analysis determines that a physical barrier is very unlikely to be present between previous first locations 10a1 and 10a2 on the second floor layout and therefore the first first user previous different level likelihood (second floor layout) for previous first locations 10a1 and 10a2 is very low. Based on the first first user previous different level likelihood for all of the floor layouts (e.g., the first floor layout and the second floor layout), an overall first first user previous different level likelihood for previous first locations 10a1 and 10a2 is determined to be very low. The different level analysis then derives a second first user previous different level likelihood (first floor layout) for previous first location 10a2 and first location 10a3. In doing so, the different level analysis determines that a physical barrier is very unlikely to be present between the previous first location 10a2 and first location 10a3 on the first floor layout and therefore the second first user previous different level likelihood (first floor layout) for previous first location 10a2 and first location 10a3 is very low. The different level analysis then derives, based on the second floor layout, a second first user previous different level likelihood (second floor layout) for previous first location 10a2 and first location 10a3. In doing so, the different level analysis determines that a physical barrier is very unlikely to be present between previous first location 10a2 and first location 10a3 on the second floor layout and therefore the second first user previous different level likelihood (second floor layout) for previous first location 10a2 and first location 10a3 is very low. Based on the second first user previous different level likelihood for all of the floor layouts (e.g., the first floor layout and the second floor layout), an overall second first user previous different level likelihood for previous first location 10a2 and first location 10a3 is determined to be very low. The different level analysis then derives a first first user subsequent different level likelihood (first floor layout) for first location 10a3 and subsequent first location 10a4. In doing so, the different level analysis determines that a physical barrier is very unlikely to be present between the first location 10a3 and subsequent first location 10a4 on the first floor layout and therefore the first first user subsequent different level likelihood (first floor layout) for first location 10a3 and subsequent first location 10a4 is very low. The different level analysis then derives, based on the second floor layout, a first first user subsequent different level likelihood (second floor layout) for first location 10a3 and subsequent first location 10a4. In doing so, the different level analysis determines that a physical barrier is very unlikely to be present between first location 10a3 and subsequent first location 10a4 on the second floor layout and therefore the first first user subsequent different level likelihood (second floor layout) for first location 10a3 and subsequent first location 10a4 is very low. Based on the first first user subsequent different level likelihood for all of the floor layouts (e.g., the first floor layout and the second floor layout), an overall first first user subsequent different level likelihood for first location 10a3 and subsequent first location 10a4 is determined to be very low. The different level analysis then derives a first first user subsequent different level likelihood (first floor layout) for first location 10a3 and subsequent first location 10a4. In doing so, the different level analysis determines that a physical barrier is very unlikely to be present between the first location 10a3 and subsequent first location 10a4 on the first floor layout and therefore the first first user subsequent different level likelihood (first floor layout) for first location 10a3 and subsequent first location 10a4 is very low. The different level analysis then derives, based on the second floor layout, a second first user subsequent different level likelihood (second floor layout) for subsequent first location 10a4 and subsequent first location 10a5. In doing so, the different level analysis determines that a physical barrier is very unlikely to be present between subsequent first location 10a4 and subsequent first location 10a5 on the second floor layout and therefore the second first user subsequent different level likelihood (second floor layout) for subsequent first location 10a4 and subsequent first location 10a5 is very low. Based on the second first user subsequent different level likelihood for all of the floor layouts (e.g., the first floor layout and the second floor layout), an overall second first user subsequent different level likelihood for subsequent first location 10a4 and subsequent first location 10a5 is determined to be very low. The different level analysis processor 247 then performs the different level analysis for the second user 10 for second location b3, previous second location b1, previous second location b2, subsequent second location b4, and subsequent second location b5 in a similar manner as described above for the first user 10. For this example, the overall first second user previous different level likelihood, the overall second second user previous different level likelihood, the overall first second user subsequent different level likelihood, and the overall second second user subsequent different level likelihood are the same as those for the overall first first user previous different level likelihood, the overall second first user previous different level likelihood, the overall first first user subsequent different level likelihood, and the overall second first user subsequent different level likelihood, respectively. Based on the overall first first user previous different level likelihood, the overall second first user previous different level likelihood, the overall first first user subsequent different level likelihood, the overall second first user subsequent different level likelihood, the overall first second user previous different level likelihood, the overall second second user previous different level likelihood, the overall first second user subsequent different level likelihood, and the overall second second user subsequent different level likelihood, the different level likelihood for the first candidate contact instance 16 may be determined to be medium or 50% (e.g., there are 4 possible scenarios (first user 10 is on floor/level 1 and second user 10 is on floor/level 1, first user 10 is on floor/level 1 and second user 10 is on floor level 2, first user 10 is on floor/level 2 and second user 10 is on floor/level 1, and first user 10 is on floor/level 2 and second user 10 is on floor/level 2), and 2 of the 4 possible scenarios results in both first and second users 10 being on the same floor/level). Accordingly, the non-contact score for the first contact instance 16 will be medium or 50% and the risk or likelihood of the second user being physically exposed to the first user (or vice versa) will be medium or 50%.

In a second example, a different level analysis is first performed on the first floor layout (e.g., floor 1F) illustrated in FIG. 7E and second floor layout (e.g., floor 2F) illustrated in FIG. 7F for first location 10e3 (i.e., first geolocation 11 for the first user) and second location 10f3 (i.e., second geolocation 11 for the second user). The different level analysis includes deriving a first first user previous different level likelihood (first floor layout) for previous first location 10e1 and previous first location 10e2. In doing so, the different level analysis determines that a physical barrier is very unlikely to be present between the previous first locations 10e1 and 10e2 on the first floor layout and therefore the first first user previous different level likelihood (first floor layout) for previous first locations 10e1 and 10e2 is very low. The different level analysis then derives, based on the second floor layout, a first first user previous different level likelihood (second floor layout) for previous first locations 10e1 and 10e2. In doing so, the different level analysis determines that a physical barrier is very unlikely to be present between previous first locations 10e1 and 10e2 on the second floor layout and therefore the first first user previous different level likelihood (second floor layout) for previous first locations 10e1 and 10e2 is very low. Based on the first first user previous different level likelihood for all of the floor layouts (e.g., the first floor layout and the second floor layout), an overall first first user previous different level likelihood for previous first locations 10e1 and 10e2 is determined to be very low. The different level analysis then derives a second first user previous different level likelihood (first floor layout) for previous first location 10e2 and first location 10e3. In doing so, the different level analysis determines that a physical barrier is very unlikely to be present between the previous first location 10e2 and first location 10e3 on the first floor layout and therefore the second first user previous different level likelihood (first floor layout) for previous first location 10e2 and first location 10e3 is very low. The different level analysis then derives, based on the second floor layout, a second first user previous different level likelihood (second floor layout) for previous first location 10e2 and first location 10e3. In doing so, the different level analysis determines that a physical barrier is very likely to be present between previous first location 10e2 and first location 10e3 on the second floor layout and therefore the second first user previous different level likelihood (second floor layout) for previous first location 10e2 and first location 10e3 is very high. The different level analysis also determines, among other things, the second second user previous different level likelihood (second floor layout) for previous second location 10f2 and second location 10f3 to be very high. In this regard, since the movement from the first previous location 10e2 to first location 10e3 for the first user 10 and the movement from the second previous location 10f2 to second location 10f3 for the second user 10 are not possible for the second floor layout, the only possible floor layout that allows for such movements for the first user 10 and the second user 10 is the first floor layout. Accordingly, the different level likelihood for the first candidate contact instance 16 is determined to be very low. Accordingly, the non-contact score for the first contact instance 16 will be very low, and the risk or likelihood of the second user being physically exposed to the first user (or vice versa) will be very high.

In a third example, a different level analysis is first performed on the first floor layout (e.g., floor 1F) illustrated in FIG. 7E and second floor layout (e.g., floor 2F) illustrated in FIG. 7F for first location 10c3 (i.e., first geolocation 11 for the first user) and second location 10d3 (i.e., second geolocation 11 for the second user). The different level analysis includes deriving a first first user previous different level likelihood (first floor layout) for previous first location 10c1 and previous first location 10c2. In doing so, the different level analysis determines that a physical barrier is very unlikely to be present between the previous first locations 10c1 and 10c2 on the first floor layout and therefore the first first user previous different level likelihood (first floor layout) for previous first locations 10c1 and 10c2 is very low. The different level analysis then derives, based on the second floor layout, a first first user previous different level likelihood (second floor layout) for previous first locations 10c1 and 10c2. In doing so, the different level analysis determines that a physical barrier is very unlikely to be present between previous first locations 10c1 and 10c2 on the second floor layout and therefore the first first user previous different level likelihood (second floor layout) for previous first locations 10c1 and 10c2 is very low. Based on the first first user previous different level likelihood for all of the floor layouts (e.g., the first floor layout and the second floor layout), an overall first first user previous different level likelihood for previous first locations 10c1 and 10c2 is determined to be very low. The different level analysis then derives a second first user previous different level likelihood (first floor layout) for previous first location 10c2 and first location 10c3. In doing so, the different level analysis determines that a physical barrier is very unlikely to be present between the previous first location 10c2 and first location 10c3 on the first floor layout and therefore the second first user previous different level likelihood (first floor layout) for previous first location 10c2 and first location 10c3 is very low. The different level analysis then derives, based on the second floor layout, a second first user previous different level likelihood (second floor layout) for previous first location 10c2 and first location 10c3. In doing so, the different level analysis determines that a physical barrier is very likely to be present between previous first location 10c2 and first location 10c3 on the second floor layout and therefore the second first user previous different level likelihood (second floor layout) for previous first location 10c2 and first location 10c3 is very high. For the second user 10, the different level analysis determines the first second user previous different level likelihood (first floor layout) for previous second locations 10d1 and 10d2 to be very low (i.e., second user stationary or not moving) and the second second user previous different level likelihood (second floor layout) for previous second location 10d2 and second location 10d3 to be very low (i.e., second user stationary or not moving). However, the different level analysis will determine the second second subsequent different level likelihood (first floor layout) for the subsequent second locations 10d4 and 10d5 to be very high based on a determination that a physical barrier is identified between the subsequent second locations 10d4 and 10d5. In this regard, since the movement from the first previous location 10c2 to first location 10c3 for the first user 10 is not possible in the second floor layout (i.e., physical barrier), the movement from the second previous location 10d2 to second location 10d3 for the second user 10 is possible (i.e., stationary or not moving) in the second floor layout, the movement from the second subsequent location 10d4 to second subsequent location 10d5 for the second user 10 is not possible in the first floor layout (i.e., physical barrier), and the movement from the second subsequent location 10d4 to second subsequent location 10d5 for the second user 10 is possible in the second floor layout, the only possible floor layout that allows for movements for the first user 10 is the first floor layout and the only possible floor layout that allows for movements for the second user 10 is the second floor layout. Put differently, the first user 10 is identified to be on floor 1F and the second user 10 is identified to be on floor 2F. Accordingly, the different level likelihood for the first candidate contact instance 16 is determined to be very high, the non-contact score for the first contact instance 16 is determined to be very high, and the risk or likelihood of the second user 10 being physically exposed to the first user 10 (or vice versa) is determined to be very low.

As another example (not shown), the different level analysis processor 247 may determine, based on a first geolocation 11 (for a first user 10) and a second geolocation 11 (for a second user 10) of a first candidate contact instance 16, that the first geolocation 11 and second geolocation are in a multi-level building (e.g., 10 level or floor building). The different level analysis processor 247 then obtains floor layouts for all 10 floors of the multi-level building. For each of the floor layouts, the different level analysis processor 247 performs processing (e.g., based on previous first geolocations 11 in previous first user information sets 15 for the first user 10, the first geolocation 11 for the first user 10, and/or subsequent first geolocations 11 in subsequent first user information sets 15 for the first user 10, and such processing may include deriving first first user previous different level likelihood, second first user previous different level likelihood, first first user subsequent different level likelihood, second first user subsequent different level likelihood, and/or one or more other information described above and in the present disclosure) to identify, from among the 10 floor layouts, which of the floor layouts have a likelihood (exceeding a threshold value) that the first user 10 was on the floor/level corresponding to that floor layout during the first candidate contact instance 16. For example, based on the processing of the 10 floor layouts, the different level analysis processor 247 may identify that there is a likelihood (e.g., exceeding a threshold value) that the first user 10 was on floors/levels 2, 3, and 5 (i.e., floor layouts for floors/levels 2, 3, and 5 were identified as being likely) during the first candidate contact instance 16. Similarly, for each of the floor layouts, the different level analysis processor 247 performs processing (e.g., based on previous second geolocations 11 in previous second user information sets 15 for the second user 10, the second geolocation 11 for the second user 10, and/or subsequent second geolocations 11 in subsequent second user information sets 15 for the second user 10, and such processing may include deriving first second user previous different level likelihood, second second user previous different level likelihood, first second user subsequent different level likelihood, second second user subsequent different level likelihood, and/or one or more other information described above and in the present disclosure) to identify, from among the 10 floor layouts, which of the floor layouts have a likelihood (exceeding a threshold value) that the second user 10 was on the floor/level corresponding to that floor layout during the first candidate contact instance 16. For example, based on the processing of the 10 floor layouts, the different level analysis processor 247 may identify that there is a likelihood (e.g., exceeding a threshold value) that the second user 10 was on floors/levels 4, 5, 8, and 10 (i.e., floor layouts for floors/levels 4, 5, 8, and 10 were identified as being likely) during the first candidate contact instance 16. In this particular example, there may be at least 12 possible scenarios for the first and second users (e.g., first user 10 is on floor/level 2 and second user 10 is on floor/level 4, first user 10 is on floor/level 2 and second user 10 is on floor/level 5, first user 10 is on floor/level 2 and second user 10 is on floor/level 8, first user 10 is on floor/level 2 and second user 10 is on floor/level 10, first user 10 is on floor/level 3 and second user 10 is on floor/level 4, first user 10 is on floor/level 3 and second user 10 is on floor/level 5, first user 10 is on floor/level 3 and second user 10 is on floor/level 8, first user 10 is on floor/level 3 and second user 10 is on floor/level 10, first user 10 is on floor/level 5 and second user 10 is on floor/level 4, first user 10 is on floor/level 5 and second user 10 is on floor/level 5, first user 10 is on floor/level 5 and second user 10 is on floor/level 8, and first user 10 is on floor/level 5 and second user 10 is on floor/level 10). In this particular example, since there may only be one scenario from among the at least 12 possible scenarios in which both first and second users 10 are on the same floor/level (i.e., first and second users 10 are on floor/level 5), the different level likelihood for the first candidate contact instance 16 may be determined to be very high (e.g., 1 in 12 or 8.33% chance of being on same level/floor, or 11 in 12 or 91.67% chance of being on different levels/floors), the non-contact score for the first contact instance 16 is determined to be very high, and the risk or likelihood of the second user 10 being physically exposed to the first user 10 (or vice versa) is determined to be very low. The layout analysis processor 242 may also be used to assess a first physical barrier likelihood for floor/level 5 to further refine the non-contact score.

In a similar example to the one above, the different level analysis processor 247 may determine, based on a first geolocation 11 (for a first user 10) and a second geolocation 11 (for a second user 10) of a first candidate contact instance 16, that the first geolocation 11 and second geolocation are in a multi-level building (e.g., 10 level or floor building). Based on the processing of the 10 floor layouts, the different level analysis processor 247 may identify that there is a likelihood (exceeding a threshold value) that the first user 10 was on floors/levels 2, 4, 5, and 8 (i.e., floor layouts for floors/levels 2, 4, 5, and 8 were identified as being likely) during the first candidate contact instance 16. The different level analysis processor 247 may also identify that there is a likelihood (exceeding a threshold value) that the second user 10 was on floors/levels 4, 5, 8, and 10 (i.e., floor layouts for floors/levels 4, 5, 8, and 10 were identified as being likely) during the first candidate contact instance 16. In this particular example, there may be at least 16 possible scenarios for the first and second users (e.g., first user 10 is on floor/level 2 and second user 10 is on floor/level 4, first user 10 is on floor/level 2 and second user 10 is on floor/level 5, first user 10 is on floor/level 2 and second user 10 is on floor/level 8, first user 10 is on floor/level 2 and second user 10 is on floor/level 10, first user 10 is on floor/level 4 and second user 10 is on floor/level 4, first user 10 is on floor/level 4 and second user 10 is on floor/level 5, first user 10 is on floor/level 4 and second user 10 is on floor/level 8, first user 10 is on floor/level 4 and second user 10 is on floor/level 10, first user 10 is on floor/level 5 and second user 10 is on floor/level 4, first user 10 is on floor/level 5 and second user 10 is on floor/level 5, first user 10 is on floor/level 5 and second user 10 is on floor/level 8, first user 10 is on floor/level 5 and second user 10 is on floor/level 10, first user 10 is on floor/level 8 and second user 10 is on floor/level 4, first user 10 is on floor/level 8 and second user 10 is on floor/level 5, first user 10 is on floor/level 8 and second user 10 is on floor/level 8, and first user 10 is on floor/level 8 and second user 10 is on floor/level 10). In this particular example, since there may be three scenario from among the at least 16 possible scenarios in which both first and second users 10 are on the same floor/level (i.e., first and second users 10 are both on floors/levels 4, 5, or 8), the different level likelihood for the first candidate contact instance 16 may be determined to be high (e.g., 3 in 16 or 18.75% chance of being on same level/floor, or 13 in 16 or 81.25% chance of being on different levels/floors), the non-contact score for the first contact instance 16 is determined to be high, and the risk or likelihood of the second user 10 being physically exposed to the first user 10 (or vice versa) is determined to be low. The layout analysis processor 242 may also be used to assess a first physical barrier likelihood for floor/level 5 to further refine the non-contact score.

After the different level likelihood is derived/generated (including one or more of the components or considerations of the different level likelihood, as described above and in the present disclosure), the different level analysis processor 247 is configurable or configured to obtain a threshold different level value from the threshold different level generator 206d (e.g., via the main threshold processor 202′). The threshold different level value may be dynamically generated by the threshold different level generator 206d based on one or more considerations including, but not limited to, one or more of the components or considerations of the different level likelihood, one or more aspects of the first candidate contact instance 16, etc. Alternatively or in addition, the threshold different level value may be a default or predetermined threshold value (or selected from one or more default and/or predetermined threshold values). The different level analysis processor 247 is then configurable or configured to compare the different level likelihood with the threshold different level value.

In an example embodiment, the different level analysis processor 247 is then configurable or configured to provide the different level likelihood for the first candidate contact instance 16 to the non-contact score generator 248 for use in deriving the non-contact score for the first candidate contact instance 16 (e.g., along with one or more results from other elements of the non-contact processor 240, including the layout analysis processor 242, map analysis processor 243, the first transportation-based analysis processor 244, the second transportation-based analysis processor 245, and/or the third transportation-based analysis processor 246). In example embodiments, the different level analysis processor 247 provides the different level likelihood to the non-contact score generator 248 based on a comparison of the different level likelihood with the threshold different level value. For example, the different level analysis processor 247 provides the different level likelihood to the non-contact score generator 248 when the different level likelihood is greater than or equal to the threshold different level value.

viii. The Non-Contact Score Generator (e.g., Element 248).

As illustrated in FIG. 6, an example embodiment of the non-contact processor 240 includes one or more non-contact score generators (e.g., non-contact score generators 248). The non-contact score generators 248 is configurable or configured to receive results from one or more elements of the non-contact processor 240. For example, for each candidate contact instance 16, the non-contact score generators 248 may receive a first physical barrier likelihood and/or other information from the layout analysis processor 242. Alternatively or in addition, for each candidate contact instance 16, the non-contact score generators 248 may also receive a second physical barrier likelihood and/or other information from the map analysis processor 243. Alternatively or in addition, for each candidate contact instance 16, the non-contact score generators 248 may also receive a vehicular separation likelihood and/or other information from the first transportation-based analysis processor 244. Alternatively or in addition, for each candidate contact instance 16, the non-contact score generators 248 may also receive a first different transport mode likelihood and/or other information from the second transportation-based analysis processor 245. Alternatively or in addition, for each candidate contact instance 16, the non-contact score generators 248 may also receive a second different transport mode likelihood and/or other information from the second transportation-based analysis processor 246. Alternatively or in addition, for each candidate contact instance 16, the non-contact score generators 248 may also receive a different level likelihood and/or other information from the different levels analysis processor 247.

Based on the one or more likelihoods received by the non-contact score generator 248, as described above and in the present disclosure, the non-contact score generator 248 is then configurable or configured to generate a non-contact score for each candidate contact instance 16. For each candidate contact instance 16, a high non-contact score represents a high likelihood that the candidate contact instance 16 did not result in a physical exposure between the users 10 (i.e., the first user 10 and second user 10 in the candidate contact instance 16). For each candidate contact instance 16, a low non-contact score represents a low likelihood the candidate contact instance 16 did not result in a physical exposure between the users 10 (i.e., the first user 10 and the second user 10 in the candidate contact instance 16); or put differently, a high likelihood the candidate contact instance 16 resulted in a physical exposure between the users 10.

The results of the non-contact score generator, including the non-contact score for each candidate contact instance 16, is then provided to the contact instance selector 236.

The Contact Instance Selector (e.g., Element 236).

As illustrated in FIG. 5, an example embodiment of the physical exposure processor 230 includes one or more contact instance selectors (e.g., contact instance selectors 236). The contact instance selector 236 is configurable or configured to receive candidate contact instances 16. The contact instance selector 236 is also configurable or configured to receive non-contact scores for each candidate contact instance 16 from the non-contact processor 240. The contact instance selector 236 may also receive inaccuracy scores for each candidate contact instance 16 from the inaccuracy processor 234. The contact instance selector 236 is also configurable or configured to receive threshold physical exposure values for each candidate contact instance 16 from the threshold physical exposure generator 207 (e.g., via the main threshold processor 202′).

In an example embodiment, the threshold physical exposure value may be dynamically generated by the threshold physical exposure generator 207 based on one or more considerations including, but not limited to, whether the non-contact score and/or the inaccuracy score is being used, one or more results from the various elements of the non-contact processor 240, one or more aspects of the candidate contact instance 16, etc. Alternatively or in addition, the threshold physical exposure value may be a default or predetermined threshold value (or selected from one or more default and/or predetermined threshold values).

The contact instance selector 236 is then configurable or configured to select, from among the candidate contact instances 16 received, selected contact instances. In an example embodiment, the contact instance selector 236 selects a candidate contact instance 16 to be a selected contact instance based on a comparison of the non-contact score for the candidate contact instance 16 and the threshold physical exposure value for the candidate contact instance 16. For example, the contact instance selector 236 selects a candidate contact instance 16 to be a selected contact instance when the non-contact score for the candidate contact instance 16 is greater or equal to the threshold physical exposure value for the candidate contact instance 16. In another example embodiment, the contact instance selector 236 selects a candidate contact instance 16 to be a selected contact instance based on a comparison of the inaccuracy score for the candidate contact instance 16 and the threshold physical exposure value for the candidate contact instance 16. For example, the contact instance selector 236 selects a candidate contact instance 16 to be a selected contact instance when the inaccuracy score for the candidate contact instance 16 is greater or equal to the threshold physical exposure value for the candidate contact instance 16. In another example embodiment, the contact instance selector 236 selects a candidate contact instance 16 to be a selected contact instance based on a comparison of the non-contact score and the inaccuracy score for the candidate contact instance 16 and the threshold physical exposure value for the candidate contact instance 16. For example, the contact instance selector 236 selects a candidate contact instance 16 to be a selected contact instance when the non-contact score and the inaccuracy score for the candidate contact instance 16 are greater or equal to the threshold physical exposure value for the candidate contact instance 16.

Selected contact instances selected by the contact instance selector 236 are then provided to the contact instance notification processor 250.

The Contact Instance Notification Processor (e.g., Element 250).

As illustrated in FIG. 2, an example embodiment of the processor 200 includes one or more contact instance notification processors (e.g., contact instance notification processors 250). The contact instance notification processor 250 (and/or the main interface 201) is configurable or configured to send information and/or notifications to users 10, designated devices installed at particular locations 11′, the network 20, databases 30, and/or administrators 40 and/or trusted authorities 40.

For selected contact instances selected by the contact instance selector 236 (i.e., candidate contact instances 16 in which the second user 10 is identified as having a risk of being physically exposed to the first user 10 (e.g., when the non-contact score for the candidate contact instance 16 for the second user 10 and the first user 10 is equal to or below a threshold non-contact score)), the contact instance notification processor 250 is configurable or configured to perform, among other things, one or more of the following: send a notification to an administrator 40 and/or trusted authority 40 (e.g., government entity) regarding the risk of physical exposure between a first user 10 and a second user 10 (e.g., when the first user 10 has been identified as a particular user, or vice versa); send a notification to the second user 10 to inform the second user 10 of his/her risk of being physically exposed to the first user 10 (who is a particular user) (the identity of the first user 10 may or may not be disclosed; in preferred embodiments, the identity of the first user 10 may remain anonymized from the perspective of the second user 10), and such notification may also include other information such as instructions on what to do, where to seek treatment, how to self-isolate, possible symptoms (in some example embodiments, such a notification, when received by a user 10, results in an indication or status for the user 10 (e.g., in a specialized mobile application on the user's mobile phone, such as one required by a governmental entity and/or one that is optional but may be required to be shown before entry by certain entities or companies (such as employers, office buildings, restaurants, businesses, etc.)) that can only be cleared or changed by an administrator 40 and/or trusted authority 40 (which may only occur if the user 10 has received testing and/or treatment or performed required self-isolation and/or quarantine)); send a notification to the first user 10 to inform the first user 10 that he/she may have infected another user 10 (the identity of the second user 10 may or may not be disclosed; in preferred embodiments, the identity of the second user 10 may remain anonymized from the perspective of the first user 10); generate a location-based information set 15′ (similar to a user information set 15, but for a location and not for a particular user 10) for a particular location 11′ (e.g., address, building name, landmark, business name, set of geolocations, etc.) matching or including the geolocations 11 of the candidate first user information set 15 and the candidate second user information set 15; etc.

In example embodiments where the unique identifier 13 for a user 10 (e.g., the first user 10, the second user 10, etc.) is anonymized in an information set 15 and/or contact instance 16 (e.g., anonymized via hashing, encryption, etc.), the contact instance notification processor 250 is configurable or configured to send information and/or notifications (as described above and in the present disclosure), including the notifications to users 10 (e.g., the first user 10, the second user 10, other users 10, etc.) and/or notifications to administrator 40 and/or trusted authority 40 (e.g., government entity) in one or more ways, including, but not limited to: de-anonymize and/or cooperate with an administrator 40 and/or trusted authority 40 to de-anonymize the anonymized unique identifier 13 (e.g., an administrator 40 and/or trusted authority 40 possesses and/or has access to encryption keys, private keys, passwords, etc.); access and/or cooperate with an administrator 40 and/or trusted authority 40 to access a secure database 30, blockchain 30, or the like, that cross-references and/or links the anonymized unique identifier 13 to a user contact information (e.g., email address, user ID for a dedicated mobile application installed on each user device, mobile number, dedicated address or identifier for a dedicated mobile application installed on each user device, etc.; including an anonymized version of one or more of these); etc.

While various embodiments in accordance with the disclosed principles have been described above, it should be understood that they have been presented by way of example only, and are not limiting. Thus, the breadth and scope of the example embodiments described in the present disclosure should not be limited by any of the above-described exemplary embodiments, but should be defined only in accordance with the claims and their equivalents issuing from this disclosure. Furthermore, the above advantages and features are provided in described embodiments, but shall not limit the application of such issued claims to processes and structures accomplishing any or all of the above advantages.

For example, as referred to herein, a controller, processor, and/or device may be any computing device or communication device, and may include a virtual machine, computer, node, instance, host, or machine in a networked computing environment. Also as referred to herein, a network or cloud may be a collection of machines connected by communication channels that facilitate communications between machines and allow for machines to share resources. Network may also refer to a communication medium between processes on the same machine. Also as referred to herein, a network element, node, or server may be a machine deployed to execute a program operating as a socket listener and may include software instances.

Memory (or storage or database) may comprise any collection and arrangement of volatile and/or non-volatile components suitable for storing data. For example, memory may comprise random access memory (RAM) devices, read-only memory (ROM) devices, magnetic storage devices, optical storage devices, and/or any other suitable data storage devices. In particular embodiments, memory may represent, in part, computer-readable storage media on which computer instructions and/or logic are encoded. Memory may represent any number of memory components within, local to, and/or accessible by a processor.

Various terms used herein have special meanings within the present technical field. Whether a particular term should be construed as such a “term of art” depends on the context in which that term is used. Terms are to be construed in light of the context in which they are used in the present disclosure and as one of ordinary skill in the art would understand those terms in the disclosed context. Definitions provided herein are not exclusive of other meanings that might be imparted to those terms based on the disclosed context.

Words of comparison, measurement, and timing such as “at the time”, “equivalent”, “during”, “complete”, and the like should be understood to mean “substantially at the time”, “substantially equivalent”, “substantially during”, “substantially complete”, etc., where “substantially” means that such comparisons, measurements, and timings are practicable to accomplish the implicitly or expressly stated desired result.

Additionally, the section headings and topic headings herein are provided for consistency with the suggestions under various patent regulations and practice, or otherwise to provide organizational cues. These headings shall not limit or characterize the embodiments set out in any claims that may issue from this disclosure. Specifically, a description of a technology in the “Background” is not to be construed as an admission that technology is prior art to any embodiments in this disclosure. Furthermore, any reference in this disclosure to “invention” in the singular should not be used to argue that there is only a single point of novelty in this disclosure. Multiple inventions may be set forth according to the limitations of the claims issuing from this disclosure, and such claims accordingly define the invention(s), and their equivalents, that are protected thereby. In all instances, the scope of such claims shall be considered on their own merits in light of this disclosure, but should not be constrained by the headings herein.

1. A method of managing contact tracing information, the method comprising:
receiving, by a processor, a plurality of first user information sets, each of the first user information sets including a geolocation of a first user device of a first user and a corresponding timestamp for the geolocation of the first user device;
receiving, by the processor, a plurality of other user information sets, including receiving a plurality of first other user information sets, wherein each of the first other user information sets include a geolocation of a first other user device of a first other user and a corresponding timestamp for the geolocation of the first other user device;
performing, by the processor, a candidate contact process, the candidate contact process including:
generating one or more candidate contact instances, including a first candidate contact instance, the first candidate contact instance including a first candidate first user information set and a first candidate first other user information set, the first candidate first user information set being one of the plurality of first user information sets, the first candidate first other user information set being one of the plurality of first other user information sets, wherein the first candidate first user information set and the first candidate first other user information set are selected from among the plurality of first user information sets and the plurality of first other user information sets, respectively, based on at least the following conditions:
a distance between a geolocation in the first candidate first user information set and a geolocation in the first candidate first other user information set is less than a threshold distance value; and
an amount of time between a timestamp in the first candidate first user information set and a timestamp in the first candidate first other user information set is less than a threshold time value;
performing, by the processor, a physical exposure assessment process for each of the one or more candidate contact instances, the physical exposure assessment process including:
deriving a non-contact score for each of the one or more candidate contact instances, including a first non-contact score for the first candidate contact instance, the first non-contact score for the first candidate contact instance representing a likelihood that the first user and the first other user were not physically exposed to one another during the first candidate contact instance, the deriving of the first non-contact score including:
performing a layout analysis;
performing a first map analysis;
performing a first transportation-based separation analysis;
performing a second transportation-based separation analysis; and
performing a third transportation-based separation analysis;
wherein the layout analysis includes:
searching for a first floor layout, the first floor layout being a floor layout for a geographical area covering the geolocation in the first candidate first user information set and the geolocation in the first candidate first other user information set;
identifying a first location on the first floor layout representing the geolocation in the first candidate first user information set;
identifying a second location on the first floor layout representing the geolocation in the first candidate first other user information set;
analyzing a first area on the first floor layout, the first area on the first floor layout including at least an area between the first and second locations on the first floor layout, the analyzing of the first area on the first floor layout including determining a likelihood that a first physical barrier is represented in the first area on the first floor layout; and
deriving a first physical barrier likelihood, the first physical barrier likelihood being a likelihood that the first physical barrier was present between the geolocation in the first candidate first user information set and the geolocation in the first candidate first other user information set during the first candidate contact instance, the first physical barrier likelihood derived based on the analyzing of the first area on the first floor layout;
wherein the first map analysis includes:
searching for a first map, the first map being a map for a geographical area covering the geolocation in the first candidate first user information set and the geolocation in the first candidate first other user information set;
identifying a first location on the first map representing the geolocation in the first candidate first user information set;
identifying a second location on the first map representing the geolocation in the first candidate first other user information set;
analyzing a first area on the first map, the first area on the first map including at least an area between the first and second locations on the first map, the analyzing of the first area on the first map including determining a likelihood that a second physical barrier is represented in the first area on the first map; and
deriving a second physical barrier likelihood, the second physical barrier likelihood being a likelihood that the second physical barrier was present between the geolocation in the first candidate first user information set and the geolocation in the first candidate first other user information set during the first candidate contact instance, the second physical barrier likelihood derived based on the analyzing of the first area on the first map;
wherein the first transportation-based separation analysis includes:
searching for the first map;
deriving a first transport area likelihood, the first transport area likelihood being a likelihood that the geolocation in the first candidate first user information set is within a first transport area on the first map, the first transport area being an area on the first map representing an area intended as a vehicle transportation area;
deriving a second transport area likelihood, the second transport area likelihood being a likelihood that the geolocation in the first candidate first other user information set is within a second transport area on the first map, the second transport area being an area on the first map representing an area intended as a vehicle transportation area; and
deriving a vehicular separation likelihood, the vehicular separation likelihood being a likelihood that the first user and/or the first other user were in a vehicle during the first candidate contact instance, the vehicular separation likelihood derived based on the first transport area likelihood and the second transport area likelihood;
wherein the second transportation-based separation analysis includes:
obtaining one or more previous first user information sets and/or one or more subsequent first user information sets, wherein a timestamp of each of the one or more previous first user information sets are timestamps that precede the timestamp of the first candidate first user information set, wherein a timestamp of each of the one or more subsequent first user information sets are timestamps that are after the timestamp of the first candidate first user information set;
obtaining one or more previous first other user information sets and/or one or more subsequent first other user information sets, wherein a timestamp of each of the one or more previous first other user information sets are timestamps that precede the timestamp of the first candidate first other user information set, wherein a timestamp of each of the one or more subsequent first other user information sets are timestamps that are after the timestamp of the first candidate first other user information set;
deriving a travel speed of the first user device based on:
the first candidate first user information set; and/or
the one or more previous first user information sets; and/or
the one or more subsequent first user information sets;
deriving a travel speed of the first other user device based on:
the first candidate first other user information set; and/or
the one or more previous first other user information sets; and/or
the one or more subsequent first other user information sets; and
deriving a first different transport mode likelihood, the first different transport mode likelihood being a likelihood that the first user and the first other user were traveling via a different transport mode during the first candidate contact instance, the first different transport mode likelihood derived based on a comparison of the derived travel speed of the first user device and the derived travel speed of the first other user device;
wherein the third transportation-based separation analysis includes:
obtaining the one or more previous first user information sets and/or the one or more subsequent first user information sets;
obtaining the one or more previous first other user information sets and/or the one or more subsequent first other user information sets;
deriving a travel direction of the first user device based on:
the first candidate first user information set; and/or
the one or more previous first user information sets; and/or
the one or more subsequent first user information sets;
deriving a travel direction of the first other user device based on:
the first candidate first other user information set; and/or
the one or more previous first other user information sets; and/or
the one or more subsequent first other user information sets; and
deriving a second different transport mode likelihood, the second different transport mode likelihood being a likelihood that the first user and the first other user were traveling via a different transport mode during the first candidate contact instance, the second different transport mode likelihood derived based on a comparison of the derived travel direction of the first user device and the derived travel direction of the first other user device;
wherein the first non-contact score for the first candidate contact instance is derived based on at least one of the following: the first physical barrier likelihood, the second physical barrier likelihood, the vehicular separation likelihood, the first different transport mode likelihood, and/or the second different transport mode likelihood; and
selecting the first candidate contact instance as a contact instance in which there is a risk of physical exposure when the first non-contact score is below a threshold non-contact score.

2. The method of claim 1, further comprising:
identifying, by the processor, two consecutive first user information sets from among the plurality of first user information sets, the two consecutive first user information sets including a first consecutive first user information set and a second consecutive first user information set, the first consecutive first user information set and second consecutive first user information set consecutively generated by the first user device, the first consecutive first user information set having a first geolocation and a first timestamp, the second consecutive first user information set having a second geolocation and a second timestamp;
generating, by the processor, one or more first interpolated first user information sets, each first interpolated first user information set having a first interpolated timestamp and a first interpolated geolocation, each first interpolated timestamp being a timestamp selected between the first timestamp and the second timestamp, each first interpolated geolocation being a geolocation that has been interpolated based on at least the first interpolated timestamp;
identifying, by the processor, two consecutive first other user information sets from among the plurality of first other user information sets, the two consecutive first other user information sets consecutively generated by the first other user device, the two consecutive first other user information sets including a first consecutive first other user information set and a second consecutive first other user information set, the first consecutive first other user information set having a third geolocation and a third timestamp, the second consecutive first other user information set having a fourth geolocation and a fourth timestamp; and
generating, by the processor, one or more first interpolated first other user information sets, each first interpolated first other user information set having a second interpolated timestamp and a second interpolated geolocation, each second interpolated timestamp being a timestamp selected between the third timestamp and the fourth timestamp, each second interpolated geolocation being a geolocation that has been interpolated based on at least the second interpolated timestamp;
the generating of the one or more candidate contact instances includes a consideration of one or more of the first interpolated first user information sets; and/or
the generating of the one or more candidate contact instances includes a consideration of one or more of the first interpolated first other user information sets; and/or
the first candidate first user information set is one of the first interpolated first user information sets generated by the processor; and/or
the first candidate first other user information set is one of the first interpolated first other user information sets generated by the processor.

3-7. (canceled)

8. The method of claim 1, wherein the deriving of the first non-contact score is also based on:
performing an analysis to determine whether the geolocation of the first candidate first user information set and the geolocation of the first candidate first other user information set are in a multi-level building;
responsive to a determination that the geolocation of the first candidate first user information set and geolocation of the first candidate first other user information set are in a multi-level building:
searching, in a floor layout database, for one or more floor layouts for the multi-level building, each of the one or more floor layouts being a floor layout covering the geolocation of the first candidate first user information set and the geolocation of the first candidate first other user information set;
for each of the one or more floor layouts:
identifying a third location on the floor layout representing the geolocation in the first candidate first user information set;
identifying a fourth location on the floor layout representing the geolocation in the first candidate first other user information set;
identifying one or more other third locations on the floor layout based on one or more other first user information sets, the one or more other third locations including:
one or more previous third locations on the floor layout, the one or more previous third locations identified based on the one or more previous first user information sets; and/or
one or more subsequent third locations on the floor layout, the one or more subsequent third locations identified based on the one or more subsequent first user information sets; and
identifying one or more other fourth locations on the floor layout based on one or more other first other user information sets, the one or more other fourth locations including:
one or more previous fourth locations on the floor layout, the one or more previous fourth locations identified based on the one or more previous first other user information sets; and/or
one or more subsequent fourth locations on the floor layout, the one or more subsequent fourth locations identified based on the one or more subsequent first other user information sets;
selecting, based on the identifying of the third location and the one or more other third locations, one or more first select floor layouts, each first select floor layout being a floor layout in which there is no physical barrier found, by the processor, in a travel path of the first user, the travel path of the first user determined based on the third location and the one or more other third locations;
selecting, based on the identifying of the fourth location and the one or more other fourth locations, one or more second select floor layouts, each second select floor layout being a floor layout in which there is no physical barrier found, by the processor, in a travel path of the first other user, the travel path of the first other user determined based on the fourth location and the one or more other fourth locations;
comparing the one or more first select floor layouts with the one or more second select floor layouts;
deriving a different level likelihood, the different level likelihood being a likelihood that the first user and the first other user were on different levels of the multi-level building during the first candidate contact instance, the different level likelihood derived based on the comparing of the one or more first select floor layouts with the one or more second select floor layouts;
wherein the deriving of the first non-contact score for the first candidate contact instance is further based on the different level likelihood.

9-20. (canceled)

21. A method of managing contact tracing information, the method comprising:
receiving, by a processor, a candidate contact instance, the candidate contact instance including:
a first candidate first user information set for the first user, the first candidate first user information set including a geolocation and corresponding timestamp for the first user; and
a first candidate second user information set for the second user, the first candidate second user information set including a geolocation and corresponding timestamp for the second user;
performing, by the processor, a physical exposure assessment process for the candidate contact instance, the physical exposure assessment process including:
deriving a first non-contact score for the candidate contact instance, the first non-contact score for the candidate contact instance representing a likelihood that the second user was not physically exposed to the first user during the candidate contact instance, the first non-contact score derived based on:
performing an analysis to determine whether the geolocation of the first candidate first user information set and the geolocation of the first candidate second user information set are in a multi-level building;
responsive to a determination that the geolocation of the first candidate first user information set and the geolocation of the first candidate second user information set are in a multi-level building:
searching, in a floor layout database, for one or more floor layouts based on the geolocation of the first candidate first user information set and the geolocation of the first candidate second user information set;
for each of the one or more floor layouts:
identifying a first location on the floor layout representing the geolocation in the first candidate first user information set;
identifying a second location on the floor layout representing the geolocation in the first candidate second user information set;
identifying one or more other first locations on the floor layout based on one or more other first user information sets, the one or more other first locations including:
one or more previous first locations on the floor layout, the one or more previous first locations representing one or more locations of the first user prior to the first location; and/or
one or more subsequent first locations on the floor layout, the one or more subsequent first locations representing one or more locations of the first user subsequent to the first location; and
identifying one or more other second locations on the floor layout based on one or more other second user information sets, the one or more other second locations including:
one or more previous second locations on the floor layout, the one or more previous second locations representing one or more locations of the second user prior to the second location; and/or
one or more subsequent second locations on the floor layout, the one or more subsequent second locations representing one or more locations of the second user subsequent to the second location;
selecting, based on the identifying of the first location and the one or more other first locations, one or more first select floor layouts, each first select floor layout being a floor layout in which there is no physical barrier found, by the processor, in a travel path of the first user, the travel path of the first user determined based on the first location and the one or more other first locations;
selecting, based on the identifying of the second location and the one or more other second locations, one or more second select floor layouts, each second select floor layout being a floor layout in which there is no physical barrier found, by the processor, in a travel path of the second user, the travel path of the second user determined based on the second location and the one or more other second locations;
comparing the one or more first select floor layouts with the one or more second select floor layouts; and
deriving a different level likelihood, the different level likelihood being a likelihood that the first user and the second user were on different levels of the multi-level building during the candidate contact instance, the different level likelihood derived based on the comparing of the one or more first select floor layouts with the one or more second select floor layouts;
wherein the first non-contact score for the candidate contact instance is determined based on the different level likelihood; and
identifying the second user as having a risk of being physically exposed to the first user when the first non-contact score is below a threshold non-contact score.

22. The method of claim 21, further comprising:
identifying, by the processor, two consecutive first user information sets that have been consecutively generated by the first user device, the two consecutive first user information sets including a first consecutive first user information set and a second consecutive first user information set, the first consecutive first user information set having a first geolocation and a first timestamp, the second consecutive first user information set having a second geolocation and a second timestamp;
generating, by the processor, one or more first interpolated first user information sets, each first interpolated first user information set having a first interpolated timestamp and a first interpolated geolocation, each first interpolated timestamp being a timestamp selected between the first timestamp and the second timestamp, each first interpolated geolocation being a geolocation that has been interpolated based on at least the first interpolated timestamp;
identifying, by the processor, two consecutive second user information sets that have been consecutively generated by the second user device, the two consecutive second user information sets including a first consecutive second user information set and a second consecutive second user information set, the first consecutive second user information set having a third geolocation and a third timestamp, the second consecutive second user information set having a fourth geolocation and a fourth timestamp; and
generating, by the processor, one or more first interpolated second user information sets, each first interpolated second user information set having a second interpolated timestamp and a second interpolated geolocation, each second interpolated timestamp being a timestamp selected between the third timestamp and the fourth timestamp, each second interpolated geolocation being a geolocation that has been interpolated based on at least the second interpolated timestamp.

23. The method of claim 22, wherein one or more of the following apply:
the first candidate first user information set is one of the first interpolated first user information sets generated by the processor; and/or
the first candidate second user information set is one of the first interpolated second user information sets generated by the processor.

24. (canceled)

25. The method of claim 21, further comprising:
responsive to a determination that a first first select floor layout from among the first select floor layouts and a first second select floor layout from among the second select floor layouts are a same floor layout and for a same level in the multi-level building:
performing a layout analysis for the first first select floor layout for the candidate contact instance, the layout analysis including:
identifying a first location on the first first select floor layout representing the geolocation in the first candidate first user information set;
identifying a second location on the first first select floor layout representing the geolocation in the first candidate second user information set;
analyzing a first area on the first first select floor layout, the first area on the first first select floor layout including at least an area between the first and second locations on the first first select floor layout, the analyzing of the first area on the first first select floor layout including determining a likelihood that a first physical barrier is represented in the first area on the first first select floor layout; and
deriving a first physical barrier likelihood, the first physical barrier likelihood being a likelihood that the first physical barrier was present between the geolocation in the first candidate first user information set and the geolocation in the first candidate second user information set during the candidate contact instance, the first physical barrier likelihood derived based on the analyzing of the first area on the first first select floor layout;
wherein the first non-contact score for the candidate contact instance is further based on the first physical barrier likelihood.

26. A method of managing contact tracing information, the method comprising:
receiving, by a processor from a first mobile device of a first user, a plurality of first user information sets, each of the first user information sets including a geolocation of the first mobile device and a corresponding timestamp for the geolocation of the first mobile device;
receiving, by the processor from a second mobile device of a second user, a plurality of second user information sets, each second user information set including a geolocation of the second mobile device and a corresponding timestamp for the geolocation of the second mobile device;
performing, by the processor, a candidate contact process, the candidate contact process including:
selecting a first candidate first user information set and a first candidate second user information set from among the plurality of first user information sets and the plurality of second user information sets, respectively, based on at least the following conditions:
a distance between a geolocation in the first candidate first user information set and a geolocation in the first candidate second user information set is less than a threshold distance value; and
an amount of time between a timestamp in the first candidate first user information set and a timestamp in the first candidate second user information set is less than a threshold time value; and
generating a first candidate contact instance, the first candidate contact instance including the first candidate first user information set and the first candidate second user information set;
performing, by the processor, a physical exposure assessment process for the first candidate contact instance, the physical exposure assessment process including:
deriving a first non-contact score for the first candidate contact instance, the first non-contact score for the first candidate contact instance representing a likelihood that the second user was not physically exposed to the first user during the first candidate contact instance, the first non-contact score derived based on an assessment of whether or not a physical barrier was present between the geolocation in the first candidate first user information set and the geolocation in the first candidate second user information set during the first candidate contact instance; and
selecting the first candidate contact instance as a contact instance in which there is a risk of physical exposure when the first non-contact score is below a threshold non-contact score.

27. The method of claim 26, further comprising:
identifying, by the processor, two consecutive first user information sets from among the plurality of first user information sets, the two consecutive first user information sets including a first consecutive first user information set and a second consecutive first user information set, the two consecutive first user information sets consecutively generated by the first user device, the first consecutive first user information set having a first geolocation and a first timestamp, the second consecutive first user information set having a second geolocation and a second timestamp;
generating, by the processor, one or more first interpolated first user information sets, each first interpolated first user information set having a first interpolated timestamp and a first interpolated geolocation, each first interpolated timestamp being a timestamp selected between the first timestamp and the second timestamp, each first interpolated geolocation being a geolocation that has been interpolated based on at least the first interpolated timestamp;
identifying, by the processor, two consecutive second user information sets that have been consecutively generated by the second user device, the two consecutive second user information sets including a first consecutive second user information set and a second consecutive second user information set, the first consecutive second user information set having a third geolocation and a third timestamp, the second consecutive second user information set having a fourth geolocation and a fourth timestamp; and
generating, by the processor, one or more first interpolated second user information sets, each first interpolated second user information set having a second interpolated timestamp and a second interpolated geolocation, each second interpolated timestamp being a timestamp selected between the third timestamp and the fourth timestamp, each second interpolated geolocation being a geolocation that has been interpolated based on at least the second interpolated timestamp.

28. The method of claim 27, wherein one or more of the following apply:
the generating of the first candidate contact instance includes a consideration of one or more of the first interpolated first user information sets; and/or
the generating of the first candidate contact instance includes a consideration of one or more of the first interpolated second user information sets; and/or
the first candidate first user information set is one of the first interpolated first user information sets generated by the processor; and/or
the first candidate second user information set is one of the first interpolated second user information sets generated by the processor.

29. The method of claim 27, wherein:
the first interpolated geolocation and the first interpolated timestamp of each first interpolated first user information set is generated by the processor and not the first user device; and
the second interpolated geolocation and the second interpolated timestamp of each first interpolated second user information set is generated by the processor and not the second user device.

30. The method of claim 26, wherein the physical exposure assessment process includes:
receiving a request to perform a contact trace for the first user, the first user being identified as a particular user of interest;
responsive to a determination that the first non-contact score is below the threshold non-contact score:
generating a first notification to the second user, the first notification being a notification indicating the second user has a high risk of having been physically exposed to the first user; and/or
generating a second notification to the first user, the second notification being a notification indicating the first user has a high risk of having been physically exposed to the second user; and/or
generating a third notification to a trusted authority, the third notification being a notification indicating the first user and the second user have a high risk of having been physically exposed to one another, the third notification including one or more information of the candidate contact instance used to derive the first non-contact score.

31. The method of claim 26, wherein the physical exposure assessment process includes:
performing a layout analysis, the layout analysis including:
searching for a first floor layout, the first floor layout being a floor layout for a geographical area covering the geolocation in the first candidate first user information set and the geolocation in the first candidate second user information set;
identifying a first location on the first floor layout representing the geolocation in the first candidate first user information set;
identifying a second location on the first floor layout representing the geolocation in the first candidate second user information set;
analyzing a first area on the first floor layout, the first area on the first floor layout including at least an area between the first and second locations on the first floor layout, the analyzing of the first area on the first floor layout including determining a likelihood that a first physical barrier is represented in the first area on the first floor layout; and
deriving a first physical barrier likelihood, the first physical barrier likelihood being a likelihood that the first physical barrier was present between the geolocation in the first candidate first user information set and the geolocation in the first candidate second user information set during the first candidate contact instance, the first physical barrier likelihood derived based on the analyzing of the first area on the first floor layout;
wherein the first non-contact score for the first candidate contact instance is determined based on the first physical barrier likelihood.

32. The method of claim 26, wherein the physical exposure assessment process includes:
performing an analysis to determine whether the geolocation of the first candidate first user information set and the geolocation of the first candidate second user information set are in a multi-level building;
responsive to a determination that the geolocation of the first candidate first user information set and geolocation of the first candidate second user information set are in a multi-level building:
searching, in a floor layout database, for one or more floor layouts for the multi-level building, each of the one or more floor layouts being a floor layout covering the geolocation of the first candidate first user information set and the geolocation of the first candidate second user information set;
for each of the one or more floor layouts:
identifying a third location on the floor layout representing the geolocation in the first candidate first user information set;
identifying a fourth location on the floor layout representing the geolocation in the first candidate second user information set;
identifying one or more other third locations on the floor layout based on one or more other first user information sets, the one or more other third locations including:
one or more previous third locations on the floor layout, the one or more previous third locations identified based on one or more previous first user information sets, wherein a timestamp of each of the one or more previous first user information sets are timestamps that precede the timestamp of the first candidate first user information set; and/or
one or more subsequent third locations on the floor layout, the one or more subsequent third locations identified based on one or more subsequent first user information sets, wherein a timestamp of each of the one or more subsequent first user information sets are timestamps that are after the timestamp of the first candidate first user information set; and
identifying one or more other fourth locations on the floor layout based on one or more other second user information sets, the one or more other fourth locations including:
one or more previous fourth locations on the floor layout, the one or more previous fourth locations identified based on the one or more previous second user information sets, wherein a timestamp of each of the one or more previous second user information sets are timestamps that precede the timestamp of the first candidate second user information set; and/or
one or more subsequent fourth locations on the floor layout, the one or more subsequent fourth locations identified based on the one or more subsequent second user information sets, wherein a timestamp of each of the one or more subsequent second user information sets are timestamps that are after the timestamp of the first candidate second user information set;
selecting, based on the identifying of the third location and the one or more other third locations, one or more first select floor layouts, each first select floor layout being a floor layout in which there is no physical barrier found, by the processor, in a travel path of the first user, the travel path of the first user determined based on the third location and the one or more other third locations;
selecting, based on the identifying of the fourth location and the one or more other fourth locations, one or more second select floor layouts, each second select floor layout being a floor layout in which there is no physical barrier found, by the processor, in a travel path of the second user, the travel path of the second user determined based on the fourth location and the one or more other fourth locations;
comparing the one or more first select floor layouts with the one or more second select floor layouts;
deriving a different level likelihood, the different level likelihood being a likelihood that the first user and the second user were on different levels of the multi-level building during the first candidate contact instance, the different level likelihood derived based on the comparing of the one or more first select floor layouts with the one or more second select floor layouts;
wherein the deriving of the first non-contact score for the first candidate contact instance is further based on the different level likelihood.

33. The method of claim 26, wherein the physical exposure assessment process includes:
performing a first map analysis, the first map analysis including:
searching for a first map, the first map being a map for a geographical area covering the geolocation in the first candidate first user information set and the geolocation in the first candidate second user information set;
identifying a first location on the first map representing the geolocation in the first candidate first user information set;
identifying a second location on the first map representing the geolocation in the first candidate second user information set;
analyzing a first area on the first map, the first area on the first map including at least an area between the first and second locations on the first map, the analyzing of the first area on the first map including determining a likelihood that a second physical barrier is represented in the first area on the first map; and
deriving a second physical barrier likelihood, the second physical barrier likelihood being a likelihood that the second physical barrier was present between the geolocation in the first candidate first user information set and the geolocation in the first candidate second user information set during the first candidate contact instance, the second physical barrier likelihood derived based on the analyzing of the first area on the first map;
wherein the first non-contact score for the first candidate contact instance is determined based on the second physical barrier likelihood.

34. The method of claim 26, wherein the physical exposure assessment process includes:
performing a first transportation-based separation analysis, the first transportation-based separation analysis including:
searching for a first map, the first map being a map for a geographical area covering the geolocation in the first candidate first user information set and the geolocation in the first candidate second user information set;
deriving a first transport area likelihood, the first transport area likelihood being a likelihood that the geolocation in the first candidate first user information set is within a first transport area on the first map, the first transport area being an area on the first map representing an area intended as a vehicle transportation area;
deriving a second transport area likelihood, the second transport area likelihood being a likelihood that the geolocation in the first candidate second user information set is within a second transport area on the first map, the second transport area being an area on the first map representing an area intended as a vehicle transportation area; and
deriving a vehicular separation likelihood, the vehicular separation likelihood being a likelihood that the first user and/or the second user were in a vehicle during the first candidate contact instance, the vehicular separation likelihood derived based on the first transport area likelihood and the second transport area likelihood;
wherein the first non-contact score for the first candidate contact instance is determined based on the vehicular separation likelihood.

35. The method of claim 26, wherein the physical exposure assessment process includes:
performing a second transportation-based separation analysis, the second transportation-based separation analysis including:
obtaining one or more previous first user information sets and/or one or more subsequent first user information sets, wherein a timestamp of each of the one or more previous first user information sets are timestamps that precede the timestamp of the first candidate first user information set, wherein a timestamp of each of the one or more subsequent first user information sets are timestamps that are after the timestamp of the first candidate first user information set;
obtaining one or more previous second user information sets and/or one or more subsequent second user information sets, wherein a timestamp of each of the one or more previous second user information sets are timestamps that precede the timestamp of the first candidate second user information set, wherein a timestamp of each of the one or more subsequent second user information sets are timestamps that are after the timestamp of the first candidate second user information set;
deriving a travel speed of the first user device based on:
the first candidate first user information set; and/or
the one or more previous first user information sets; and/or
the one or more subsequent first user information sets;
deriving a travel speed of the second user device based on:
the first candidate second user information set; and/or
the one or more previous second user information sets; and/or
the one or more subsequent second user information sets; and
deriving a first different transport mode likelihood, the first different transport mode likelihood being a likelihood that the first user and the second user were traveling via a different transport mode during the first candidate contact instance, the first different transport mode likelihood derived based on a comparison of the derived travel speed of the first user device and the derived travel speed of the second user device;
wherein the first non-contact score for the first candidate contact instance is determined based on the first different transport mode likelihood.

36. The method of claim 35, wherein:
when a difference between the derived travel speeds of the first user and the second user is high, the first different transport mode likelihood is high; and
when a difference between the derived travel speeds of the first user and the second user is low, the first different transport mode likelihood is low.

37. The method of claim 26, wherein the physical exposure assessment process includes:
performing a third transportation-based separation analysis, the third transportation-based separation analysis including:
obtaining one or more previous first user information sets and/or one or more subsequent first user information sets, wherein a timestamp of each of the one or more previous first user information sets are timestamps that precede the timestamp of the first candidate first user information set, wherein a timestamp of each of the one or more subsequent first user information sets are timestamps that are after the timestamp of the first candidate first user information set;
obtaining one or more previous second user information sets and/or one or more subsequent second user information sets, wherein a timestamp of each of the one or more previous second user information sets are timestamps that precede the timestamp of the first candidate second user information set, wherein a timestamp of each of the one or more subsequent second user information sets are timestamps that are after the timestamp of the first candidate second user information set;
deriving a travel direction of the first user device based on:
the first candidate first user information set; and/or
the one or more previous first user information sets; and/or
the one or more subsequent first user information sets;
deriving a travel direction of the second user device based on:
the first candidate second user information set; and/or
the one or more previous second user information sets; and/or
the one or more subsequent second user information sets; and
deriving a second different transport mode likelihood, the second different transport mode likelihood being a likelihood that the first user and the second user were traveling via a different transport mode during the first candidate contact instance, the second different transport mode likelihood derived based on a comparison of the derived travel direction of the first user device and the derived travel direction of the second user device;
wherein the first non-contact score for the first candidate contact instance is determined based on the second different transport mode likelihood.

38. The method of claim 37, wherein:
when a difference between the derived travel directions of the first user and the second user is high, the second different transport mode likelihood is high; and
when a difference between the derived travel directions of the first user and the second user is low, the second different transport mode likelihood is low.

39-52. (canceled)
